SEQUENCE LISTING (55 sequences)

SEQ ID NO: 1 - DNA, 2254 bp, Homo sapiens
  CDS (517)..(1782)
ctcgctcctctcctacttggataactgtggtaattctagagctaatacatgccgacgggcgctgacccccttcgcgggggggatgcgtgcatttatcagatcaagaccaacccggtcagcccctctccggccccggccggggggcgggcgccggcggctttggtgactctagataacctcgggccgatcgcacgccccccgtggcggcgacgacccattcgaacgtctgccctatcaactttcgatggtagtcgccgtgcctaccatggtgaccacgggtgacggggaatcagggttcgattccggagagggagcctgagaaacggctaccacatccaaggaaggcagcaggcgcgcaaattacccactcccggaggtggcggcggcgccatcttggcgaaggggggatcaggaagtgcggaccgcggcggcggcggcggcggcggcggcggcggagcccggagcgcaggccggaggctcccggcccgccggccccggagcggagcggagcggaggatgcagcagccgcagccgcaggggcagcagcagccggggccggggcagcagctggggggccagggggcggcgccgggggccgggggcggcccaggggggggcccggggccggggccctgcctgaggcgagagctgaagctgctcgagtccatcttccaccgcggccacgagcgcttccgcattgccagcgcctgcctggacgagctgagctgcgagttcctgctggctggggccggaggggccggggcgggggccgcgcccggaccgcatctccccccacgggggtcggtgcctggggatcctgtccgcatccactgcaacatcacggagtcataccctgctgtgccccccatctggtcggtggagtctgatgaccctaacttggctgctgtcttggagaggctggtggacataaagaaagggaatactctgctattgcagcatctgaagaggatcatctccgacctgtgtaaactctataacctccctcagcatccagatgtggagatgctggatcaacccttgccagcagagcagtgcacacaggaagacgtgtcttcagaagatgaagatgaggagatgcctgaggacacagaagacttagatcactatgaaatgaaagaggaagagccagctgagggcaagaaatctgaagatgatggcattggaaaagaaaacttggccatcctagagaaaattaaaaagaaccagaggcaagattacttaaatggtgcagtgtctggctcggtgcaggccactgaccggctgatgaaggagctcagggatatataccgatcacagagtttcaaaggcggaaactatgcagtcgaactcgtgaatgacagtctgtatgattggaatgtcaaactcctcaaagttgaccaggacagcgctttgcacaacgatctccagatcctcaaagagaaagaaggagccgacttcattctacttaacttttcctttaaagataactttccctttgacccaccatttgtcagggttgtgtctccagtcctctctggagggtatgttctgggcggaggggccatctgcatggaacttctcaccaaacagggctggagcagtgcctactccatagagtcagtgatcatgcagatcagtgccacactggtgaaggggaaagcacgagtgcagtttggagccaacaaatctcaatacagtctgacaagagcacagcagtcctacaagtccttggtgcagatccacgaaaaaaacggctggtacacacccccaaaagaagacggctaaccctggagtatcacccttcctccctccccaggcaccactggaccaattacctttgaatgctgtatttggatctcacgctgcctctgtggttccctccctcatttttcctggacgtgatagctctgcctattgcaggacaatgatggctattctaaacgctaaggaaaaaaaacaaacacagaactgtttcaagtactcaagactgacttacagaccaaccaaccaccttgctggaacccttgctagcaggcattcttataaaagaaactttcgagcctccttatattgctggaaactcagctgtgctccagactagagcctccttacctatgctatggatttttaatttattttctcttatttcatgtacactgctttttttggttacagtgtatgatggatgtgtatgaaaaaaatgtatctttgggaaaacaattacagtttgttaatttgaaaaaaaaaaaaaaaa

SEQ ID NO: 2 - protein, 422 aa, Homo sapiens
MQQPQPQGQQQPGPGQQLGGQGAAPGAGGGPGGGPGPGPCLRRELKLLESIFHRGHERFRIASACLDELSCEFLLAGAGGAGAGAAPGPHLPPRGSVPGDPVRIHCNITESYPAVPPIWSVESDDPNLAAVLERLVDIKKGNTLLLQHLKRIISDLCKLYNLPQHPDVEMLDQPLPAEQCTQEDVSSEDEDEEMPEDTEDLDHYEMKEEEPAEGKKSEDDGIGKENLAILEKIKKNQRQDYLNGAVSGSVQATDRLMKELRDIYRSQSFKGGNYAVELVNDSLYDWNVKLLKVDQDSALHNDLQILKEKEGADFILLNFSFKDNFPFDPPFVRVVSPVLSGGYVLGGGAICMELLTKQGWSSAYSIESVIMQISATLVKGKARVQFGANKSQYSLTRAQQSYKSLVQIHEKNGWYTPPKEDG

SEQ ID NO: 3 - protein, 471 aa, Caenorhabditis elegans
MACLRKLKEDIQVLEKLFPKNHNRFQILSASVDELSMKFINAENKGIIVTANIQENYPRQPPIWFSESDDVPVIGMSLQRLTETEESTNILHQVHRLVSDLCSFYNLQMPCELPQIAPPVRDDIDEGRGSDISDTTSEPIDDDMAGDGEVDDDDEEEEDDEDADGDIEIVEMAEEDPTSQHDVGVSKEGLDMLDKVSKINRQQHLDGKVQGSITATDRLMKEIRDIHRSEHFKNGIYTFELEKEENLYQWWIKLHKVDEDSPLFEDMKKLKKDHNQDHLLFSFTFNEKFPCDPPFVRVVAPHINQGFVLGGGAICMELLTKQGWSSAYSIESCILQIAATLVKGRARISFDAKHTSTYSMARAQQSFKSLQQIHAKSGCTFLCSTPSSHFFALHLVFFLHSDDFFFNGFLKSETFTFFKLSFRGYISSLVLYSFSRHLHHPFFTRFLIPQLQPPPIPFQLIPPFLNRTKHV

SEQ ID NO: 4 - protein, 397 aa, Drosophila melanogaster
MACLNTLKQEIKTLEKIFPKNHERFQILNSSVDELLCRFIDKNGKRYDIHANITETYPSSPPVWFAESEETSVTNAVQILSNTNGRDNHVINQVGILLRELCRLHNVPLPPDIDNLALPLQTPPPSASPLRCEQRPGGGGAGGGGGPHGNEETDSDQEEIEDPIGESEQESEGDEDLPLEMDDVRSTSKKDDMEVEHLATLEKLRQSQRQDYLKGSVSGSVQATDRLMKELRDIYRSDAFKKNMYSIELVNESIYEWNIRLKSVDPDSPLHSDLQMLKEKEGKDSILLNILFKETYPFEPPFVRVVHPIISGGYVLIGGAICMELLTKQGWSSAYTVEAVIMQIAATLVKGKARIQFGATKALTQGQYSLARAQQSFKSLVQIHEKNGWFTPPKEDG

SEQ ID NO: 5 - protein, 207 aa, Mus musculus
MSSDRQRSDDESPSTSSGSSDADQRDPAAPKPEEQEERKPSATQQKKNTKLSSKTTAKLSTSAKRIQKELAEITLDPPPNCSAGPKGDNIYEWRSTILGPPGSVYEGGVFFLDITFSSDYPFKPPKVTFRTRIYHCNINSQGVICLDILKDNWSPALTISKVLLSICSLLTDCNPADPLVGSIATQYLTNRAEHDRIARQWTKRYAT

SEQ ID NO: 6 - protein, 200 aa, Homo sapiens
MANIAVQRIKREFKEVLKSEETSKNQIKVDLVDENFTELRGEIAGPPDTPYEGGRYQLEIKIPETYPFNPPKVRFITKIWHPNISSVTGAICLDILKDQWAAAMTLRTVLLSLQALLAAAEPDDPQDAVVANQYKQNPEMFKQTARLWAHVYAGAPVSSPEYTKKIENLCAMGFDRNAVIVALSSKSWDVETATELLLSN

SEQ ID NO: 7 - protein, 199 aa, Drosophila melanogaster
MANMAVSRIKREFKEVMRSEEIVQCSIKIELVNDSWTELRGEIAGPPDTPYEGGKFVLEIKVPETYPFNPPKVRFITRIWHPNISSVTGAICLDILKDNWAAAMTLRTVLLSLQALLAAAEPDDPQDAVVAYQFKDKYDLFLLTAKHWTNAYAGGPHTFPDCDSKIQRLRDMGIDEHEARAVLSKENWNLEKATEGLFS

SEQ ID NO: 8 - protein, 295 aa, Saccharomyces cerevisiae
MSSRKSTASSLLLRQYRELTDPKKAIPSFHIELEDDSNIFTWNIGVMVLNEDSIYHGGFFKAQMRFPEDFPFSPPQFRFTPAIYHPNVYRDGRLCISILHQSGDPMTDEPDAETWSPVQTVESVLISIVSLLEDPNINSPANVDAAVDYRKNPEQYKQRVKMEVERSKQDIPKGFIMPTSESAYISQSKLDEPESNKDMADNFWYDSDLDDDENGSVILQDDDYDDGNNHIPFEDDDVYNYNDNDDDDERIEFEDDDDDDDDSIDNDSVMDRKQPHKAEDESEDVEDVERVSKKI

SEQ ID NO: 9 - DNA, 20 bp, Artificial Sequence
  Description of Artificial Sequence Primer
tgcagtgtctggctcggtgc

SEQ ID NO: 10 - DNA, 21 bp, Artificial Sequence
  Description of Artificial Sequence Primer
ctgatctgcatgatcactgac

SEQ ID NO: 11 - DNA, 30 bp, Artificial Sequence
  Description of Artificial Sequence Synthetic oligonucleotide
tccactgcaacatcacggagtcataccctg

SEQ ID NO: 12 - DNA, 30 bp, Artificial Sequence
  Description of Artificial Sequence Synthetic oligonucleotide
atgcagtcgaactcgtgaatgacagtctgt

SEQ ID NO: 13 - DNA, 39 bp, Artificial Sequence
  Description of Artificial Sequence Primer
gcagcagcggccgcgacgagctgagctgcgagttcctgc

SEQ ID NO: 14 - DNA, 37 bp, Artificial Sequence
  Description of Artificial Sequence Primer
gcagcagtcgacgccgtcttcttttgggggtgtgtac

SEQ ID NO: 15 - DNA, 39 bp, Artificial Sequence
  Description of Artificial Sequence Primer
gcagcagcggccgcatgcagcagccgcagccgcaggggc

SEQ ID NO: 16 - DNA, 37 bp, Artificial Sequence
  Description of Artificial Sequence Primer
gcagcagtcgacgccctgtttggtgagaagttccatg

SEQ ID NO: 17 - protein, 245 aa, Homo sapiens
PHLPPRGSVPGDPVRIHCNITESYPAVPPIWSVESDDPNLAAVLERLVDIKKGNTLLLQHLKRIISDLCKLYNLPQHPDVEMLDQPLPAEQCTQEDVSSEDEDEEMPEDTEDLDHYEMKEEEPAEGKKSEDDGIGKENLAILEKIKKNQRQDYLNGAVSGSVQATDRLMKELRDIYRSQSFKGGNYAVELVNDSLYDWNVKLLKVDQDSALHNDLQILKEKEGADFILLNFSFKDNFPFDPPFVR

SEQ ID NO: 18 - protein, 13 aa, Homo sapiens
GSVQATDRLMKEL

SEQ ID NO: 19 - protein, 13 aa, Homo sapiens
IYRSQSFKGGNYA

SEQ ID NO: 20 - protein, 13 aa, Homo sapiens
ILLNFSFKDNFPF

SEQ ID NO: 21 - protein, 13 aa, Homo sapiens
TRAQQSYKSLVQI

SEQ ID NO: 22 - protein, 14 aa, Homo sapiens
PAEQCTQEDVSSED

SEQ ID NO: 23 - protein, 14 aa, Homo sapiens
TQEDVSSEDEDEEM

SEQ ID NO: 24 - protein, 14 aa, Homo sapiens
QEDVSSEDEDEEMP

SEQ ID NO: 25 - protein, 14 aa, Homo sapiens
AEGKKSEDDGIGKE

SEQ ID NO: 26 - protein, 14 aa, Homo sapiens
ELVNDSLYDWNVKL

SEQ ID NO: 27 - protein, 14 aa, Homo sapiens
ILLNFSFKDNFPFD

SEQ ID NO: 28 - protein, 14 aa, Homo sapiens
VRIHCNITESYPAV

SEQ ID NO: 29 - protein, 14 aa, Homo sapiens
AVELVNDSLYDWNV

SEQ ID NO: 30 - protein, 14 aa, Homo sapiens
DFILLNFSFKDNFP

SEQ ID NO: 31 - protein, 14 aa, Homo sapiens
VQFGANKSQYSLTR

SEQ ID NO: 32 - protein, 16 aa, Homo sapiens
QQPGPGQQLGGQGAAP

SEQ ID NO: 33 - protein, 16 aa, Homo sapiens
PGQQLGGQGAAPGAGG

SEQ ID NO: 34 - protein, 16 aa, Homo sapiens
QLGGQGAAPGAGGGPG

SEQ ID NO: 35 - protein, 16 aa, Homo sapiens
AAPGAGGGPGGGPGPG

SEQ ID NO: 36 - protein, 16 aa, Homo sapiens
APGAGGGPGGGPGPGP

SEQ ID NO: 37 - protein, 16 aa, Homo sapiens
EFLLAGAGGAGAGAAP

SEQ ID NO: 38 - protein, 16 aa, Homo sapiens
LLAGAGGAGAGAAPGP

SEQ ID NO: 39 - protein, 16 aa, Homo sapiens
LAGAGGAGAGAAPGPH

SEQ ID NO: 40 - protein, 16 aa, Homo sapiens
HLPPRGSVPGDPVRIH

SEQ ID NO: 41 - protein, 16 aa, Homo sapiens
QDYLNGAVSGSVQATD

SEQ ID NO: 42 - protein, 16 aa, Homo sapiens
NGAVSGSVQATDRLMK

SEQ ID NO: 43 - protein, 16 aa, Homo sapiens
SQSFKGGNYAVELVND

SEQ ID NO: 44 - protein, 16 aa, Homo sapiens
GYVLGGGAICMELLTK

SEQ ID NO: 45 - protein, 16 aa, Homo sapiens
ARVQFGANKSQYSLTR

SEQ ID NO: 46 - protein, 14 aa, Homo sapiens
EEEPAEGKKSEDDG

SEQ ID NO: 47 - protein, 164 aa, Homo sapiens
GSVQATDRLMKELRDIYRSQSFKGGNYAVELVNDSLYDWNVKLLKVDQDSALHNDLQILKEKEGADFILLNFSFKDNFPFDPPFVRVVSPVLSGGYVLGGGAICMELLTKQGWSSAYSIESVIMQISATLVKGKARVQFGANKSQYSLTRAQQSYKSLVQIHEK

SEQ ID NO: 48 - DNA, 20 bp, Artificial Sequence
  Description of Artificial Sequence Primer
aggatcatctccgacctgtg

SEQ ID NO: 49 - DNA, 20 bp, Artificial Sequence
  Description of Artificial Sequence Primer
caagggttgatccagcatct

SEQ ID NO: 50 - DNA, 20 bp, Artificial Sequence
  Description of Artificial Sequence Primer
atgaggcttggatcagcttt

SEQ ID NO: 51 - DNA, 20 bp, Artificial Sequence
  Description of Artificial Sequence Primer
cctgaagcctgacattccat

SEQ ID NO: 52 - DNA, 21 bp, Artificial Sequence
  Description of Artificial Sequence Primer
actgcagccgattcattaatg

SEQ ID NO: 53 - DNA, 48 bp, Artificial Sequence
  Description of Artificial Sequence Primer
gaattaatacgactcactatagggagatatcatacacatacgatttag

SEQ ID NO: 54 - DNA, 48 bp, Artificial Sequence
  Description of Artificial Sequence Primer
gaattaatacgactcactatagggagacatgattacgccaagctcgaa

SEQ ID NO: 55 - DNA, 21 bp, Artificial Sequence
  Description of Artificial Sequence Primer
tgtaaaacgacggccagtgaa

(19) United States
(12) Patent Application Publication
(10) Pub. No.: US 2003/0190613 A1
(43) Pub. Date: Oct. 9, 2003

(54) Polynucleotide encoding an activated human T-lymphocyte-derived protein related to ubiquitin conjugating enzyme

(76) Inventors: Michael A. Bowen, Rockville, MD (US); Yuli Wu, Newtown, PA (US); Wen-Pin Yang, Princeton, NJ (US); Joshua Finger, San Marcos, CA (US); Steven Nadler, Princeton, NJ (US); Pamela Carroll, Princeton, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/005,549

(22) Filed: Oct. 29, 2001

Related U.S. Application Data
(60) Provisional application No. 60/308,706, filed on Jul. 30, 2001.
(60) Provisional application No. 60/244,688, filed on Oct. 30, 2000.

(51) Int. Cl. C12Q 1/68; C07H 21/04; C12N 9/64; C12P 21/02; C12N 5/06

(52) U.S. Cl. 435/6; 435/69.1; 435/226; 435/320.1; 435/325; 536/23.2

(57) ABSTRACT

The present invention describes a newly discovered ubiquitin conjugating enzyme homologue, called RATL1d6 herein, and its encoding polynucleotide, isolated and identified from activated T lymphocytes. Also described are expression vectors, host cells, agonists, antagonists, antisense molecules, and antibodies associated with the activity and use of the newly-discovered polynucleotide and/or polypeptide of the present invention. Methods for treating, diagnosing, preventing and screening for disorders related to the expression of the RATL1d6 ubiquitin conjugating enzyme polypeptide are described.

This application claims benefit to provisional application U.S. Serial No. 60/308,706, filed Jul. 30, 2001 and to provisional application U.S. Serial No. 60/244,688, filed Oct. 30, 2000.

### FIELD OF THE INVENTION

The invention relates to the identification and isolation of a novel polynucleotide sequence and its encoded amino acid sequence defining a polypeptide expressed in activated human T-lymphocytes (T-cells) and having similarity to ubiquitin conjugating enzyme (UBC). The invention further relates to the use of the polynucleotides and the polypeptide in regulating cell growth and cell cycle progression, as well as in targeting the degradation of cellular proteins, and in the diagnosis, treatment and prevention of neoplastic diseases, immune disorders, and developmental and neuronal disorders and diseases.

### BACKGROUND OF THE INVENTION

The ubiquitin conjugation system (UCS) serves as a major pathway for the degradation of cellular proteins in eukaryotic cells and in some bacteria. The UCS mediates the elimination of abnormal or aberrant proteins and regulates the half-lives of important regulatory proteins that control cellular processes, such as gene transcription and cell cycle progression. The UCS is believed to be involved in the degradation of mitotic cyclic kinases, oncoproteins, tumor suppressor gene products (e.g., p53), viral proteins, cell surface receptors associated with signal transduction, transcriptional regulators and mutated or damaged proteins (A. Ciechanover, 1994, Cell, 79:13-21).

Ubiquitin conjugating enzymes (UBCs) selectively target proteins for proteosomal degradation by the covalent attachment of ubiquitin moieties. The ubiquitination of cellular proteins appears to be mediated by the specific interplay between ubiquitin conjugating enzymes (E2s) and ubiquitin protein ligases (E3s) (T. P. Moynihan et al., 1999, J. Biol. Chem., 274(43):30963-8). Ubiquitin-mediated proteolysis controls diverse physiological processes in eukaryotic and mammalian cells, including, but not limited to, DNA repair, cell cycle regulation and p53-dependent processes.

The ubiquitination pathway targets not only normal, i.e., short-lived, intracellular eukaryotic proteins for degradation when appropriate, but it also serves to eliminate abnormal, mutant, or misfolded proteins from the cell. (T. P. Moynihan et al., 1999, Mammalian Genome, 10:977-982). In the ubiquitin pathway proteins that are covalently ligated to ubiquitin are targeted for degradation by the cell. The selectivity of the destruction is ensured by the substrate specificity in the ubiquitination steps which are comprised of a series of enzymatic reactions. (F. Yamao, 1999, J. Biochem. (Tokyo), 125(2):223-9). Ubiquitin ligase (E3), in conjunction with ubiquitin conjugating enzyme (E2), have been implicated in playing an essential role in the recognition of substrate.

The process of ubiquitin conjugation and protein degradation involves four main steps (S. Jentsch, 1992, Ann. Rev. Genet., 26:179-207). In the first step, a ubiquitin activating enzyme (E1) activates ubiquitin (Ub), a small, heat stable protein (76 amino acids) in an ATP dependent reaction which binds the C-terminus of Ub to the thiol group of an internal cysteine residue in E1. Next, activated Ub is transferred to one of several Ub-conjugating enzymes (E2). Different ubiquitin-dependent proteolytic pathways employ structurally similar, but distinct, ubiquitin-conjugating enzymes that are associated with recognition subunits which direct them to proteins which carry a particular degradation signal. E2 then links the Ub molecule through its C-terminal glycine to an internal lysine (acceptor lysine) of a target protein. In some instances, accessory factors, known as ubiquitin ligases (E3s) are required to work in concert with E2s for the recognition of certain substrates. Additional Ub molecules may be added, ultimately forming a multi-Ub chain structure. The ubiquitinated protein is then recognized and degraded by the proteosome, a large multisubunit proteolytic enzyme complex, and Ub is released for reutilization.

UBCs and families of genes encoding UBCs have been identified in a variety of eukaryotic genera, e.g., Saccharomyces, Dictyostelium, Drosophila, Caenorabditis elegans (C. elegans), Paramecia, as well as in mice and humans. UBCs or E2s are encoded by a large family of genes that are related to each other.

The E2 ubiquitin-conjugating enzymes are important for substrate specificity in different UCS pathways. All E2 molecules have a conserved domain of approximately 16 kDa, called the UBC domain, that is at least 35% identical to all other E2s and contains a centrally located cysteine residue that is required for ubiquitin-enzyme thioester formation (S. Jentsch, supra). A highly conserved proline-rich element is located N-terminal to the active cysteine residue. Structural variations beyond this conserved domain are used to classify the E2 enzymes. The E2s of class I (E2-1) consist almost exclusively of the conserved UBC domain and include yeast E2-1 and UBCs 4, 5 and 7. These E2s are thought to require E3 to carry out their activities. (See, S. Jentsch, supra). UBC7 has been shown to recognize ubiquitin as a substrate and to form polyubiquitin chains in vitro (S. Van Nocker et al., 1996, J. Biol. Chem., 271:12150-12158). The E2s of class II (E2-2) have various unrelated C-terminal extensions that contribute to substrate specificity and cellular localization. The yeast E2-2 enzymes, UBC2 and UBC3, have highly acidic C-terminal extensions that promote interactions with basic substrates such as histones. Yeast UBC6 has a hydrophobic signal-anchor sequence that localizes the protein to the endoplasmic reticulum.

Defects or alterations in the normal activity of the UCS are associated with a number of diseases and disorders. These include increased ubiquitin-dependent proteolysis that is associated with cachexia (M. Llovera et al., 1995, Int. J. Cancer, 61:138-141), degradation of the tumor suppressor protein p53 (A. Ciechanover, supra), neurodegeneration, such as is observed in Alzheimer's disease (L. Gregori et al., 1994, Biochem. Biophys. Res. Comm., 203-1731-1738), and in muscle-wasting disorders, such as is observed after serious injury and in diseases such as cancer and AIDS (Sciencexpress, 2001,10:1126). Since ubiquitin conjugation is a rate-limiting step in antigen presentation, the ubiquitin degradation pathway is likely to play a role in the immune response to antigen (E. P. Grant et al., 1995, J. Immunol., 155:3750-3758). Indeed, because the ubiquitin conjugating enzyme homologue of the present invention was identified and isolated from activated T lymphocytes, a link to a role in the immune system is supported by this invention.

The discovery of new ubiquitin conjugating enzymes and the polynucleotides encoding these proteins provides the art with new compositions and methods of use and treatment for the diagnosis, screening, monitoring, therapy, and prevention of neoplastic diseases, including cancers and tumors, immune disorders, and developmental and neuronal disorders, conditions, or diseases.

### SUMMARY OF THE INVENTION

The present invention provides a novel polynucleotide encoding a ubiquitin conjugating enzyme homologue, which was isolated from activated human T-cells, and hereinafter designated RATL1d6 (“Regulated in Activated T Lymphocytes 1d6”). RATL1d6 was discovered to be upregulated upon stimulation of Jurkat-line T cells and human peripheral blood T lymphocytes with antibodies directed against the CD3 and CD28 cell surface antigens. The RATL1d6 nucleic acid was identified in a subtraction library from activated human T lymphocytes as described herein. The RATL1d6 polypeptide encoded by the RATL1d6 nucleic acid sequence provided by this invention has similarity to ubiquitin conjugating enzyme.

It is an object of the present invention to provide an isolated RATL1d6 polynucleotide as depicted in SEQ ID NO:1. In accordance with this invention, the isolated RATL1d6 polynucleotide encodes a ubiquitin conjugating enzyme comprising the amino acid sequence as set forth in SEQ ID NO:2. Fragments or portions of the RATL1d6 polynucleotide and polypeptide are also embraced by the invention. Preferably, the isolated RATL1 d6 polynucleotide or polypeptide, or fragment or portion thereof, is substantially purified.

It is another object of the present invention to provide an isolated RATL1d6 polynucleotide having the nucleic acid sequence of ATCC Deposit No. PTA-3745.

It is another object of the present invention to provide a ubiquitin conjugating enzyme homologue, the RATL1d6 polypeptide, encoded by the polynucleotide of SEQ ID NO:1 and having the amino acid sequence of SEQ ID NO:2, or a functional or biologically active portion thereof. Also provided are transmembrane domain regions of the RATL1d6 polypeptide and the encoding polynucleotides as elucidated herein.

Yet another object of the present invention is to provide a ubiquitin conjugating enzyme polypeptide comprising an amino acid sequence having at least 80% to 90% sequence identity to the sequence set forth in SEQ ID NO:2.

It is another object of the present invention to provide an isolated and substantially purified ubiquitin conjugating enzyme polypeptide encoded by the nucleic acid sequence of ATCC Deposit No. PTA-3745.

It is another object of the present invention to provide a ubiquitin conjugating enzyme polypeptide whose amino acid sequence differs from SEQ ID NO:2 only by conservative substitutions.

It is another object of the present invention to provide RATL1d6 polypeptides having N-terminal, C-terminal and internal deletions as described herein. Polynucleotides encoding these deletion polypeptides are also provided. According to the invention, such RATL1d6 polypeptides can comprise immunogenic and/or antigenic epitopes as described herein.

It is a further object of the present invention to provide the polynucleotide sequence of RATL1d6 (SEQ ID NO:1) lacking the initiating codon, as well as the resulting encoded polypeptide. In accordance with this invention, the polynucleotide corresponding to nucleotides 520 through 1782 of SEQ ID NO:1, and the polypeptide corresponding to amino acids 2 through 422 of SEQ ID NO:2 are provided.

It is yet another object of the present invention to provide a ubiquitin conjugating enzyme polypeptide having at least 80% to 95% sequence identity to the sequence as set forth in SEQ ID NO:2.

It is a further object of the present invention to provide a substantially purified ubiquitin conjugating enzyme fusion protein, wherein all or a portion of the RATL1d6 polypeptide is conjugated, coupled or linked to a heterologous polypeptide or peptide. More particularly, the invention provides an amino acid sequence having at least 80% to 95% sequence identity to the sequence as set forth in SEQ ID NO:2 and an amino acid sequence of an Fc portion of a human immunoglobulin protein. According to the present invention a ubiquitin conjugating enzyme fusion protein is provided in which the amino acid sequence differs from SEQ ID NO:2 only by conservative substitutions.

It is a further object of the present invention to provide compositions comprising the RATL1d6 polynucleotide sequence, or a fragment thereof, or the encoded RATL1d6 polypeptide, or a fragment or portion thereof. Also provided by the present invention are pharmaceutical compositions comprising at least one RATL1d6 polypeptide, or a functional portion thereof, wherein the compositions further comprise a pharmaceutically or physiologically acceptable carrier, excipient, or diluent.

It is yet another object of the present invention to provide a novel isolated and purified, preferably substantially purified, polynucleotide that encodes the RATL1d6 ubiquitin conjugating enzyme homolog. In a particular aspect, the polynucleotide comprises the nucleotide sequence of SEQ ID NO:1. The present invention also provides a polynucleotide sequence comprising the complement of SEQ ID NO:1, or variants thereof. In addition, the present invention features polynucleotide sequences which hybridize under moderately stringent or high stringency conditions to the polynucleotide sequence of SEQ ID NO:1. The present invention further provides an isolated polynucleotide comprising a nucleic acid sequence having (i) SEQ ID NO:1; (ii) a nucleic acid sequence degenerate from SEQ ID NO:1 as a result of genetic code redundancy, or (iii) a complementary nucleic acid sequence thereto. More particularly, the complementary nucleic acid sequence of (iii) can hybridize to either strand of a denatured, double-stranded polynucleotide comprising the nucleic acid sequence of SEQ ID NO:1 under conditions of moderate or high stringency, where a nonlimiting example of moderately stringent conditions comprises 50% formamide, 5× Denhart's solution, 5×SSPE or SSC, 0.2% SDS at about 42° C., followed by washing in 0.2×SSPE or SSC and 0.2% SDS at a temperature of about 42° C. to about 50° C. High stringency conditions typically permit hybridization of those nucleic acid sequences that form stable hybrids in 0.018 M NaCl at about 65° C.

It is yet another object of the present invention to provide a nucleic acid sequence encoding the RATL1d6 polypeptide and an antisense of the nucleic acid sequence, as well as oligonucleotides, fragments, or portions of the nucleic acid molecule or antisense molecule. The RATL1d6 nucleic acid sequence, particularly, oligonucleotides, fragments, or portions of the RATL1d6 nucleic acid molecule are useful as hybridization probes to detect, diagnose or monitor RATL1d6 in body fluid samples. Also provided are expression vectors and host cells comprising polynucleotides, or fragments or portions thereof, that encode the RATL1d6 polypeptide or peptides thereof.

It is also an object of the present invention to provide methods for producing a polypeptide comprising the amino acid sequence depicted in SEQ ID NO:2, or a fragment thereof, comprising: (a) cultivating a host cell containing an expression vector containing at least a functional fragment of the polynucleotide sequence encoding the RATL1d6 ubiquitin conjugating enzyme homologue according to this invention under conditions suitable for the expression of the polynucleotide; and (b) recovering the polypeptide from the host cell.

It is yet another object of the present invention to provide antibodies, and binding fragments thereof, which bind specifically to the RATL1d6 polypeptide, or an epitope thereof, for use as therapeutics and diagnostic agents.

It is also an object of the present invention to provide methods for screening for agents which bind to and/or modulate RATL1d6 polypeptide, e.g., agonists and antagonists, as well as modulators, e.g., agonists and antagonists, particularly those that are obtained from the screening methods described.

It is another object of the present invention to provide a purified, preferably, substantially purified, antagonist of the polypeptide of SEQ ID NO:2. In this regard, and by way of example, a purified antibody that binds to a polypeptide comprising the amino acid sequence of SEQ ID NO:2 is provided. In accordance with the present invention, substantially purified agonists of the polypeptide of SEQ ID NO:2 are further provided.

It is yet another object of the present invention to provide RATL1d6 nucleic acid sequences, polypeptide, peptides and antibodies for use in the diagnosis and/or screening of disorders or diseases associated with expression of the polynucleotide and its encoded polypeptide as described herein.

It is a further object of the present invention to provide kits for screening and diagnosis of disorders associated with aberrant or uncontrolled cellular development and with the expression of the polynucleotide and its encoded polypeptide as described herein.

It is another object of the present invention to provide methods for the treatment or prevention of cancers or tumors, immune disorders, lymphoproliferative disorders, or neurodegenerative disorders involving administering to an individual in need of treatment or prevention an amount of a purified antagonist or inhibitor of the RATL1d6 ubiquitin conjugating enzyme effective to treat or prevent the disease or disorder. For example, in cancer or tumor therapy methods, the ubiquitin conjugating enzyme antagonist is preferably utilized in an amount effective to block ubiquitination of a tumor suppressor gene in cancer or tumor cells.

Another object of the present invention is to provide a method of suppressing the immune response in a subject requiring immune response suppression, comprising administration of an antagonist or inhibitor of RATL1d6 ubiquitin conjugating enzyme, or an agonist or activator of RATL1d6 ubiquitin conjugating enzyme in an amount effective to cause immunosuppression. In such methods, immunosuppression can be the result of ubiquination of a cell receptor and subsequent down regulation of receptor activity.

Another object of the present invention is to provide a method of treating aberrant or abnormal cell growth causing uncontrolled proliferation of a cell in a mammal comprising administration of the RATL1d6 polypeptide or its homologue as described in accordance with the invention, or an agonist or antagonist thereof, in an amount effective to treat the abnormal or aberrant cell growth and uncontrolled cell proliferation.

It is yet another object of the present invention to provide a method of diagnosing a disease or susceptibility to a disease in a mammal related to expression or activity of ubiquitin conjugating enzyme. The method comprises contacting a sample from a mammal with an antibody specific for the RATL1d6 polypeptide, its homologue, or antigenic fragment thereof, under conditions in which an antigen-antibody complex can form between the antibody and the polypeptide, or homologue, or antigenic fragment thereof in the sample, and detecting an antigen-antibody complex, if formed. Detection of the complex indicates the presence of the RATL1d6 polypeptide, or homologue, or antigenic fragment thereof, in the sample, wherein an increased amount of complex formed with the polypeptide, homologue, or fragment thereof, in a test sample compared with the amount in a normal control sample is indicative of disease or condition, or susceptibility to a disease or condition in the mammal. According to the invention, the disease can be an immune disorder, a neuronal disorder, a developmental disorder, or neoplastic growth.

It is a further object of the present invention to provide a nucleic acid-based method of diagnosing a disease, disorder or condition, in which the method comprises hybridizing a RATL1d6 polynucleotide or fragment or oligo thereof according to the invention to the nucleic acid material of a sample, thereby forming a hybridization complex; and detecting the hybridization complex. In the method, the presence of the complex diagnoses a disease, disorder, or condition correlating with the presence of a polynucleotide encoding ubiquitin conjugating enzyme, or a fragment thereof, in the sample. Such a method can comprise in situ hybridization.

It is yet another object of the present invention to provide a method for detecting a polynucleotide that encodes the RATL1d6 polypeptide in a biological sample comprising (a) hybridizing the complement of the polynucleotide sequence encoding SEQ ID NO:2 to a nucleic acid material of a biological sample, thereby forming a hybridization complex; and (b) detecting the hybridization complex, wherein the presence of the complex correlates with the presence of a polynucleotide encoding the RATL1d6 polypeptide in the biological sample. The nucleic acid material may be further amplified by the polymerase chain reaction prior to hybridization.

Another object of the present invention provides a method of detecting ubiquitin conjugating enzyme or homologue, or an antibody-reactive fragment thereof, in a sample, comprising contacting the sample with an antibody specific for the RATL1d6 polypeptide, or an antigenic fragment thereof, under conditions in which an antigen-antibody complex can form between the antibody and the polypeptide or antigenic fragment thereof in the sample; and detecting an antigen-antibody complex formed. In such a method detection of the complex indicates the presence of ubiquitin conjugating enzyme, or an antigenic fragment thereof, in the sample.

It is a further object of the present invention to provide a method of screening a library of molecules or compounds with a polynucleotide to identify at least one molecule or compound therein which specifically binds to the polynucleotide sequence, comprising combining the RATL1d6 polynucleotide or peptide, or a bindable portion thereof peptide with a library of molecules or compounds under conditions to allow specific binding; and detecting specific binding, thereby identifying a molecule or compound which specifically binds to the polynucleotide sequence. In such a method, the library can comprise DNA molecules, RNA molecules, artificial chromosome constructions, PNAs, peptides and proteins.

It is yet another object of the present invention to provide a method of using the RATL1d6 polynucleotide sequence to purify a molecule or compound in a sample, in which the molecule or compound specifically binds to the RATL1d6 polynucleotide. The method comprises combining the RATL1d6 polynucleotide, a bindable portion thereof, or a RATL1d6 variant, under conditions to allow specific binding; detecting specific binding between the polynucleotide and the molecule or compound; recovering the bound polynucleotide; and separating the polynucleotide from the molecule or compound, thereby obtaining a purified molecule or compound.

Another object of the present invention is to provide a method of screening for candidate compounds or agents that are capable of modulating activity of a ubiquitin conjugating enzyme, such as RATL1d6. The method comprises contacting a test compound with a substantially or partially purified RATL1d6 polypeptide, peptide, or fragment; and selecting as candidate modulating compounds those test compounds that modulate activity of the polypeptide. The assay methods may be cell-based assays in which the RATL1d6 is expressed in a cell or tissue, or cell-free assays. According to the invention and as described herein, the candidate compounds are either agonists or antagonists, or agonists or activators, of ubiquitin conjugating enzyme activity. The activity of the RATL1d6 ubiquitin activating enzyme can be protein degradation associated with lymphoproliferative disorders, cancers and tumors, neuronal disorders, or developmental disorders, or ubiquitination of a cell receptor.

A further object of this invention is to provide a method of screening for or detecting candidate compounds capable of binding to the RATL1d6 ubiquitin conjugating enzyme. The method comprises contacting a test compound with a purified RATL1d6 polypeptide or a peptide thereof according to the invention and selecting as candidate compounds those test compounds that bind to the polypeptide. In such a method, the RATL1d6 polypeptide or peptide, or the candidate compounds, can be immobilized onto a solid support. Also, in the method, selection of a candidate compound can based on affinity of binding determinations by analyzing thermal unfolding curves of complexes formed between candidate compound and the polypeptide.

In the screening methods provided by the present invention, the candidate compounds can be small molecules, therapeutics, biological agents, and/or drugs. Such methods as provided by the present invention and described herein are quite suitable for being carried out via high throughput screening technology.

Further objects, features and advantages of the present invention will be better understood upon a reading of the detailed description of the invention when considered in connection with the accompanying figures/drawings.

### BRIEF DESCRIPTION OF THE FIGURES

FIGS. 1A and 1B show separately the polynucleotide sequence of the RATL1d6 ubiquitin conjugating enzyme homologue (SEQ ID NO:1), (FIG. 1A), and the deduced amino acid sequence of the encoded RATL1d6 protein (FIG. 1B). The coding sequence (CDS) of RATL1d6 is 517 to 1782 of SEQ ID NO:1.

FIGS. 2A and 2B show the polynucleotide sequence (SEQ ID NO:1) and the deduced, encoded amino acid sequence (SEQ ID NO:2) of the RATL1d6 ubiquitin conjugating enzyme homologue, a portion of which was isolated from an activated T-cell subtraction library.

FIG. 3 shows the deduced amino acid sequence of the RATL1d6 ubiquitin conjugating enzyme polypeptide (SEQ ID NO:2). The predicted molecular weight of the RATL1d6 polypeptide encoded by the polynucleotide of SEQ ID NO:1 is MW=46.1 Kd.

FIG. 4 shows an alignment of the RATL1d6 ubiquitin conjugating enzyme (UBC) with hypothetical C. elegans and Drosophila orthologs F2H2.8 (Genbank Accession No: gi|3876332; SEQ ID NO:3) and EG:25E8.2 (Genbank Accession No: gi|7290306; SEQ ID NO:4), respectively, and the E2 UBCs P52483/mouse UB6B (Genbank Accession No: gi|117850; SEQ ID NO:5); P27924/human UBC1/Huntingtin interacting protein (HIP), (Genbank Accession No: gi|14727922; SEQ ID NO:6); CAA72184/Drosophila UBCD4 (Genbank Accession No: gi|7294892; SEQ ID NO:7); and P14682/yeast UBC3/CDC34 (Genbank Accession No: gi|6320259; SEQ ID NO:8). The ubiquitin conjugating domain identified through a search of the PFAM database is boxed in RATL1d6. Residues that are identical in three or more of the aligned sequences are highlighted, and the conserved Cys residue which has been shown in UBCs to be involved in ubiquitin transfer through a thiol ester intermediate to target proteins is marked with a “ [symbol] ”. The % identity of RATL1d6 with C. elegans F2H2.8 is 42% over the entire peptide sequence; 47% with Drosophila EG:25E8.2 over the entire peptide sequence; and approximately 25% over the UBC domains only for the remaining proteins.

FIGS. 5A and 5B show a BLAST alignment between the RATL1d6 amino acid sequence and its putative Drosophila ortholog (FIG. 5A, 65% identity) and C. elegans ortholog (FIG. 5B, 61% identity). The query sequence is that of RATL1d6, while the subject sequence is the amino acid sequence, i.e., Drosophila (EG:25E8.2) or C. elegans (F25H2.8), showing a percent identity to the RATL1d6 sequence.

FIG. 6 presents an RT-PCR expression profile of RATL1d6 as described in Example 13.

### DESCRIPTION OF THE INVENTION

The present invention provides a novel isolated polynucleotide and encoded polypeptide, the expression of which is upregulated in human T lymphocytes that have been stimulated with anti-CD28 and anti-CD3 antibodies versus unstimulated T lymphocytes. This novel polypeptide is termed herein RATL1d6, an acronym for “Regulated in Activated T Lymphocytes 1d6”, and is further characterized as a ubiquitin conjugating enzyme homologue.

The following definitions are provided to more fully describe the present invention in its various aspects. The definitions are intended to be useful for guidance and elucidation, and are not intended to limit the disclosed invention or its embodiments.

Definitions

The RATL1d6 polypeptide (or protein) refers to the amino acid sequence of substantially purified RATL1d6, which may be obtained from any species, preferably mammalian, and more preferably, human, and from a variety of sources, including natural, synthetic, semi-synthetic, or recombinant. Functional fragments of the RATL1d6 polypeptide are also embraced by the present invention.

An agonist refers to a molecule which, when bound to the RATL1d6 polypeptide, or a functional fragment thereof, increases or prolongs the duration of the effect of the RATL1d6 polypeptide. Agonists may include proteins, nucleic acids, carbohydrates, or any other molecules that bind to and modulate the effect of RATL1d6 polypeptide. An antagonist refers to a molecule which, when bound to the RATL1d6 polypeptide, or a functional fragment thereof, decreases the amount or duration of the biological or immunological activity of RATL1d6 polypeptide. Antagonists may include proteins, nucleic acids, carbohydrates, antibodies, or any other molecules that decrease or reduce the effect of RATL1d6 polypeptide.

“Nucleic acid sequence”, as used herein, refers to an oligonucleotide, nucleotide, or polynucleotide, and fragments or portions thereof, and to DNA or RNA of genomic or synthetic origin which may be single- or double-stranded, and represent the sense or antisense strand. By way of nonlimiting example, fragments include nucleic acid sequences that are greater than 20-60 nucleotides in length, and preferably include fragments that are at least 70-100 nucleotides, or which are at least 1000 nucleotides or greater in length.

Similarly, “amino acid sequence” as used herein refers to an oligopeptide, peptide, polypeptide, or protein sequence, and fragments or portions thereof, and to naturally occurring or synthetic molecules. Amino acid sequence fragments are typically from about 5 to about 30, preferably from about 5 to about 15 amino acids in length and retain the biological activity or function of the RATL1d6 polypeptide.

Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule. In addition, the terms RATL1d6 polypeptide and RATL1d6 protein are used interchangeably herein to refer to the encoded product of the RATL1d6 nucleic acid sequence of the present invention.

A variant of the RATL1d6 polypeptide refers to an amino acid sequence that is altered by one or more amino acids. The variant may have “conservative” changes, wherein a substituted amino acid has similar structural or chemical properties, e.g., replacement of leucine with isoleucine. More rarely, a variant may have “nonconservative” changes, e.g., replacement of a glycine with a tryptophan. Minor variations may also include amino acid deletions or insertions, or both. Guidance in determining which amino acid residues may be substituted, inserted, or deleted without abolishing functional biological or immunological activity may be found using computer programs well known in the art, for example, DNASTAR software.

An allele or allelic sequence is an alternative form of the RATL1d6 nucleic acid sequence. Alleles may result from at least one mutation in the nucleic acid sequence and may yield altered mRNAs or polypeptides whose structure or function may or may not be altered. Any given gene, whether natural or recombinant, may have none, one, or many allelic forms. Common mutational changes which give rise to alleles are generally ascribed to natural deletions, additions, or substitutions of nucleotides. Each of these types of changes may occur alone, or in combination with the others, one or more times in a given sequence.

Altered nucleic acid sequences encoding RATL1d6 polypeptide include nucleic acid sequences containing deletions, insertions and/or substitutions of different nucleotides resulting in a polynucleotide that encodes the same or a functionally equivalent RATL1d6 polypeptide. Altered nucleic acid sequences may further include polymorphisms of the polynucleotide encoding the RATL1d6 polypeptide; such polymorphisms may or may not be readily detectable using a particular oligonucleotide probe. The encoded protein may also contain deletions, insertions, or substitutions of amino acid residues which produce a silent change and result in a functionally equivalent RATL1d6 protein. Deliberate amino acid substitutions may be made on the basis of similarity in polarity, charge, solubility, hydrophobicity, hydrophilicity, and/or the amphipathic nature of the residues, as long as the biological activity of RATL1 d6 protein is retained. For example, negatively charged amino acids may include aspartic acid and glutamic acid; positively charged amino acids may include lysine and arginine; and amino acids with uncharged polar head groups having similar hydrophilicity values may include leucine, isoleucine, and valine; glycine and alanine; asparagine and glutamine; serine and threonine; and phenylalanine and tyrosine.

“Peptide nucleic acid” (PNA) represents an oligomer of modified nucleic acid base pairs covalently linked through an amide bond. PNAs have utility in a number of antisense and anti-gene applications. These small molecules typically act by inhibiting transcription. (e.g., P. E. Nielsen et al., 1993, Anticancer Drug Des., 8:53-63). PNA may be pegylated to extend their lifespan in the cell where they preferentially bind to complementary single stranded DNA and RNA.

Oligonucleotides or oligomers refer to a nucleic acid sequence, preferably comprising contiguous nucleotides, of at least about 6 nucleotides to about 60 nucleotides, preferably at least about 8 to 10 nucleotides in length, more preferably at least about 12 nucleotides in length e.g., about 15 to 35 nucleotides, or about 15 to 25 nucleotides, or about 20 to 35 nucleotides, which can be typically used in PCR amplification assays, hybridization assays, or in microarrays. It will be understood that the term oligonucleotide is substantially equivalent to the terms primer, probe, or amplimer, as commonly defined in the art. It will also be appreciated by those skilled in the pertinent art that a longer oligonucleotide probe, or mixtures of probes, e.g., degenerate probes, can be used to detect longer, or more complex, nucleic acid sequences, for example, genomic DNA. In such cases, the probe may comprise at least 20-200 nucleotides, preferably, at least 30-100 nucleotides, more preferably, 50-100 nucleotides.

Amplification refers to the production of additional copies of a nucleic acid sequence and is generally carried out using polymerase chain reaction (PCR) technologies, which are well known and practiced in the art (See, D. W. Dieffenbach and G. S. Dveksler, 1995, PCR Primer, a Laboratory Manual, Cold Spring Harbor Press, Plainview, N.Y.).

Microarray is an array of distinct polynucleotides or oligonucleotides synthesized on a substrate, such as paper, nylon, or other type of membrane; filter; chip; glass slide; or any other type of suitable solid support.

The term antisense refers to nucleotide sequences, and compositions containing nucleic acid sequences, which are complementary to a specific DNA or RNA sequence. The term “antisense strand” is used in reference to a nucleic acid strand that is complementary to the “sense” strand. Antisense (i.e., complementary) nucleic acid molecules include PNA and may be produced by any method, including synthesis or transcription. Once introduced into a cell, the complementary nucleotides combine with natural sequences produced by the cell to form duplexes which block either transcription or translation. The designation “negative” is sometimes used in reference to the antisense strand, and “positive” is sometimes used in reference to the sense strand.

The term consensus refers to the sequence that reflects the most common choice of base or amino acid at each position among a series of related DNA, RNA, or protein sequences. Areas of particularly good agreement often represent conserved functional domains.

A deletion refers to a change in either nucleotide or amino acid sequence and results in the absence of one or more nucleotides or amino acid residues. By contrast, an insertion (also termed “addition”) refers to a change in a nucleotide or amino acid sequence that results in the addition of one or more nucleotides or amino acid residues, as compared with the naturally occurring molecule. A substitution refers to the replacement of one or more nucleotides or amino acids by different nucleotides or amino acids.

A derivative nucleic acid molecule refers to the chemical modification of a nucleic acid encoding, or complementary to, the encoded RATL1d6 polypeptide. Such modifications include, for example, replacement of hydrogen by an alkyl, acyl, or amino group. A nucleic acid derivative encodes a polypeptide which retains the essential biological and/or functional characteristics of the natural molecule. A derivative polypeptide is one which is modified by glycosylation, pegylation, or any similar process that retains the biological and/or functional or immunological activity of the polypeptide from which it is derived.

The term “biologically active”, i.e., functional, refers to a protein or polypeptide or fragment thereof having structural, regulatory, or biochemical functions of a naturally occurring molecule. Likewise, “immunologically active” refers to the capability of the natural, recombinant, or synthetic RATL1d6, or any oligopeptide thereof, to induce a specific immune response in appropriate animals or cells, for example, to generate antibodies, and to bind with specific antibodies.

The term hybridization refers to any process by which a strand of nucleic acid binds with a complementary strand through base pairing.

The term “hybridization complex” refers to a complex formed between two nucleic acid sequences by virtue of the formation of hydrogen bonds between complementary G and C bases and between complementary A and T bases. The hydrogen bonds may be further stabilized by base stacking interactions. The two complementary nucleic acid sequences hydrogen bond in an anti-parallel configuration. A hybridization complex may be formed in solution (e.g., Cₒt or Rₒt analysis), or between one nucleic acid sequence present in solution and another nucleic acid sequence immobilized on a solid support (e.g., membranes, filters, chips, pins, or glass slides, or any other appropriate substrate to which cells or their nucleic acids have been affixed).

The terms stringency or stringent conditions refer to the conditions for hybridization as defined by nucleic acid composition, salt and temperature. These conditions are well known in the art and may be altered to identify and/or detect identical or related polynucleotide sequences in a sample. A variety of equivalent conditions comprising either low, moderate, or high stringency depend on factors such as the length and nature of the sequence (DNA, RNA, base composition), reaction milieu (in solution or immobilized on a solid substrate), nature of the target nucleic acid (DNA, RNA, base composition), concentration of salts and the presence or absence of other reaction components (e.g., formamide, dextran sulfate and/or polyethylene glycol) and reaction temperature (within a range of from about 5° C. below the melting temperature of the probe to about 20° C. to 25° C. below the melting temperature). One or more factors may be varied to generate conditions, either low or high stringency, that are different from but equivalent to the aforementioned conditions.

As will be understood by those of skill in the art, the stringency of hybridization may be altered in order to identify or detect identical or related polynucleotide sequences. As will be further appreciated by the skilled practitioner, Tm can be approximated by the formulas as known in the art, depending on a number of parameters, such as the length of the hybrid or probe in number of nucleotides, or hybridization buffer ingredients and conditions (See, for example, T. Maniatis et al., Molecular Cloning: A Laboratory Manual, Cold Spring Harbor Laboratory, Cold Spring Harbor, N.Y., 1982 and J. Sambrook et al., Molecular Cloning: A Laboratory Manual, Cold Spring Harbor Laboratory, Cold Spring Harbor, N.Y., 1989; Current Protocols in Molecular Biology, Eds. F. M. Ausubel et al., Vol. 1, “Preparation and Analysis of DNA”, John Wiley and Sons, Inc., 1994-1995, Suppls. 26, 29, 35 and 42; pp. 2.10.7-2.10.16; G. M. Wahl and S. L. Berger (1987; Methods Enzymol. 152:399-407); and A. R. Kimmel, 1987; Methods of Enzymol. 152:507-511). As a general guide, Tₘ decreases approximately 1° C.-1.5° C. with every 1% decrease in sequence homology. Also, in general, the stability of a hybrid is a function of sodium ion concentration and temperature. Typically, the hybridization reaction is initially performed under conditions of low stringency, followed by washes of varying, but higher stringency. Reference to hybridization stringency, e.g., high, moderate, or low stringency, typically relates to such washing conditions.

Thus, by way of nonlimiting example, high stringency refers to conditions that permit hybridization of those nucleic acid sequences that form stable hybrids in 0.018M NaCl at about 65° C. (i.e., if a hybrid is not stable in 0.018M NaCl at about 65° C., it will not be stable under high stringency conditions). High stringency conditions can be provided, for instance, by hybridization in 50% formamide, 5× Denhart's solution, 5×SSPE (saline sodium phosphate EDTA) (1×SSPE buffer comprises 0.15 M NaCl, 10 mM Na₂HPO₄, 1 mM EDTA), (or 1×SSC buffer containing 150 mM NaCl, 15 mM Na₃ citrate·2H₂O, pH 7.0), 0.2% SDS at about 42° C., followed by washing in 1×SSPE (or saline sodium citrate, SSC) and 0.1% SDS at a temperature of at least about 42° C., preferably about 55° C., more preferably about 65° C.

Moderate stringency refers, by nonlimiting example, to conditions that permit hybridization in 50% formamide, 5× Denhart's solution, 5×SSPE (or SSC), 0.2% SDS at 42° C. (to about 50° C.), followed by washing in 0.2×SSPE (or SSC) and 0.2% SDS at a temperature of at least about 42° C., preferably about 55° C., more preferably about 65° C.

Low stringency refers, by nonlimiting example, to conditions that permit hybridization in 10% formamide, 5× Denhart's solution, 6×SSPE (or SSC), 0.2% SDS at 42° C., followed by washing in 1×SSPE (or SSC) and 0.2% SDS at a temperature of about 45° C., preferably about 50° C.

For additional stringency conditions, see T. Maniatis et al., Molecular Cloning: A Laboratory Manual, Cold Spring Harbor Laboratory, Cold Spring Harbor, N.Y. (1982). It is to be understood that the low, moderate and high stringency hybridization/washing conditions may be varied using a variety of ingredients, buffers and temperatures well known to and practiced by the skilled practitioner.

The terms complementary or complementarity refer to the natural binding of polynucleotides under permissive salt and temperature conditions by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between single stranded molecules. The degree of complementarity between nucleic acid strands has significant effects on the efficiency and strength of hybridization between nucleic acid strands. This is of particular importance in amplification reactions, which depend upon binding between nucleic acids strands, as well as in the design and use of PNA molecules.

The term homology refers to a degree of complementarity. There may be partial homology or complete homology, wherein complete homology is equivalent to identity. A partially complementary sequence that at least partially inhibits an identical sequence from hybridizing to a target nucleic acid is referred to using the functional term “substantially homologous.” The inhibition of hybridization of the completely complementary sequence to the target sequence may be examined using a hybridization assay (e.g., Southern or Northern blot, solution hybridization and the like) under conditions of low stringency.

A substantially homologous sequence or probe will compete for and inhibit the binding (i.e., the hybridization) of a completely homologous sequence or probe to the target sequence under conditions of low stringency. Nonetheless, conditions of low stringency do not permit non-specific binding; low stringency conditions require that the binding of two sequences to one another be a specific (i.e., selective) interaction. The absence of non-specific binding may be tested by the use of a second target sequence which lacks even a partial degree of complementarity (e.g., less than about 30% identity). In the absence of non-specific binding, the probe will not hybridize to the second non-complementary target sequence.

Those having skill in the art will know how to determine percent identity between/among sequences using, for example, algorithms such as those based on the CLUSTALW computer program (J. D. Thompson et al., 1994, Nucleic Acids Research, 2(22):4673-4680), or FASTDB, (Brutlag et al., 1990, Comp. App. Biosci., 6:237-245), as known in the art. Although the FASTDB algorithm typically does not consider internal non-matching deletions or additions in sequences, i.e., gaps, in its calculation, this can be corrected manually to avoid an overestimation of the % identity. CLUSTALW, however, does take sequence gaps into account in its identity calculations.

A composition comprising a given polynucleotide sequence refers broadly to any composition containing the given polynucleotide sequence. The composition may comprise a dry formulation or an aqueous solution. Compositions comprising polynucleotide sequence (SEQ ID NO:1) encoding RATL1d6 polypeptide, or fragments thereof, may be employed as hybridization probes. The probes may be stored in freeze-dried form and may be in association with a stabilizing agent such as a carbohydrate. In hybridizations, the probe may be employed in an aqueous solution containing salts (e.g., NaCl), detergents or surfactants (e.g., SDS) and other components (e.g., Denhardt's solution, dry milk, salmon sperm DNA, and the like).

The term “substantially purified” refers to nucleic acid sequences or amino acid sequences that are removed from their natural environment, isolated or separated, and are at least 60% free, preferably 75% to 85% free, and most preferably 90%, or greater, free from other components with which they are naturally associated.

The term sample, or biological sample, is meant to be interpreted in its broadest sense. A biological sample suspected of containing nucleic acid encoding RATL1d6 protein, or fragments thereof, or RATL1d6 protein itself, may comprise a body fluid, an extract from cells or tissue, chromosomes isolated from a cell (e.g., a spread of metaphase chromosomes), organelle, or membrane isolated from a cell, a cell, nucleic acid such as genomic DNA (in solution or bound to a solid support such as for Southern analysis), RNA (in solution or bound to a solid support such as for Northern analysis), cDNA (in solution or bound to a solid support), a tissue, a tissue print and the like.

Transformation refers to a process by which exogenous DNA enters and changes a recipient cell. It may occur under natural or artificial conditions using various methods well known in the art. Transformation may rely on any known method for the insertion of foreign nucleic acid sequences into a prokaryotic or eukaryotic host cell. The method is selected based on the type of host cell being transformed and may include, but is not limited to, viral infection, electroporation, heat shock, lipofection, and partial bombardment. Such “transformed” cells include stably transformed cells in which the inserted DNA is capable of replication either as an autonomously replicating plasmid or as part of the host chromosome. Transformed cells also include those cells which transiently express the inserted DNA or RNA for limited periods of time.

The term “mimetic” refers to a molecule, the structure of which is developed from knowledge of the structure of RATL1d6 protein, or portions thereof, and as such, is able to effect some or all of the actions of RATL1d6 protein.

The term “portion” with regard to a protein (as in “a portion of a given protein”) refers to fragments or segments of that protein. The fragments may range in size from four or five amino acid residues to the entire amino acid sequence minus one amino acid. Thus, a protein “comprising at least a portion of the amino acid sequence of SEQ ID NO: 2” encompasses the full-length human RATL1d6 polypeptide, and fragments thereof.

The term antibody refers to intact molecules as well as fragments thereof, such as Fab, F(ab′)₂, Fv, which are capable of binding an epitopic or antigenic determinant. Antibodies that bind to RATL1d6 polypeptides can be prepared using intact polypeptides or fragments containing small peptides of interest or prepared recombinantly for use as the immunizing antigen. The polypeptide or oligopeptide used to immunize an animal can be derived from the transition of RNA or synthesized chemically, and can be conjugated to a carrier protein, if desired. Commonly used carriers that are chemically coupled to peptides include bovine serum albumin (BSA), keyhole limpet hemocyanin (KLH), and thyroglobulin. The coupled peptide is then used to immunize the animal (e.g, a mouse, a rat, or a rabbit).

The term “humanized” antibody refers to antibody molecules in which amino acids have been replaced in the non-antigen binding regions in order to more closely resemble a human antibody, while still retaining the original binding capability, e.g., as described in U.S. Pat. No. 5,585,089 to C. L. Queen et al.

The term “antigenic determinant” refers to that portion of a molecule that makes contact with a particular antibody (i.e., an epitope). When a protein or fragment of a protein is used to immunize a host animal, numerous regions of the protein may induce the production of antibodies which bind specifically to a given region or three-dimensional structure on the protein; these regions or structures are referred to an antigenic determinants. An antigenic determinant may compete with the intact antigen (i.e., the immunogen used to elicit the immune response) for binding to an antibody.

The terms “specific binding” or “specifically binding” refer to the interaction between a protein or peptide and a binding molecule, such as an agonist, an antagonist, or an antibody. The interaction is dependent upon the presence of a particular structure (i.e., an antigenic determinant or epitope) of the protein that is recognized by the binding molecule. For example, if an antibody is specific for epitope “A”, the presence of a protein containing epitope A (or free, unlabeled A) in a reaction containing labeled “A” and the antibody will reduce the amount of labeled A bound to the antibody.

The term “correlates with expression of a polynucleotide” indicates that the detection of the presence of ribonucleic acid that is similar to SEQ ID NO:1 by Northern analysis is indicative of the presence of mRNA encoding RATL1d6 polypeptide in a sample and thereby correlates with expression of the transcript from the polynucleotide encoding the protein.

An alteration in the polynucleotide of SEQ ID NO:1 comprises any alteration in the sequence of the polynucleotides encoding RATL1d6 polypeptide, including deletions, insertions, and point mutations that may be detected using hybridization assays. Included within this definition is the detection of alterations to the genomic DNA sequence which encodes RATL1d6 polypeptide (e.g., by alterations in the pattern of restriction fragment length polymorphisms capable of hybridizing to SEQ ID NO:1), the inability of a selected fragment of SEQ ID NO:1 to hybridize to a sample of genomic DNA (e.g., using allele-specific oligonucleotide probes), and improper or unexpected hybridization, such as hybridization to a locus other than the normal chromosomal locus for the polynucleotide sequence encoding RATL1 d6 polypeptide (e.g., using fluorescent in situ hybridization (FISH) to metaphase chromosome spreads).

### DESCRIPTION OF THE PRESENT INVENTION

The present invention is based on the discovery of a novel polypeptide isolated from activated human T-cells, and designated RATL1d6 herein, that was found to be upregulated upon stimulation of Jurkat-line T cells and human peripheral blood T lymphocytes with antibodies directed against the CD3 and CD28 cell surface antigens. The invention encompasses the polynucleotide encoding the RATL1d6 polypeptide and the use of compositions comprising the RATL1d6 polynucleotide or polypeptide for the screening, diagnosis, treatment or prevention of disorders associated with aberrant or uncontrolled cellular growth and/or function, such as neoplastic diseases (e.g., cancers and tumors) and immune and neurodegenerative disorders and conditions.

The invention also encompasses the polynucleotide encoding the RATL1d6 polypeptide and the use of compositions comprising the RATL1 d6 polynucleotide or polypeptide for the screening, diagnosis, treatment or prevention of disorders associated with aberrant immune responses, such as for autoimmune conditions, degenerative conditions, cachexia, muscle degeneration, etc.

Also encompassed by the present invention are fragments or portions of the RATL1d6 polynucleotide and polypeptide sequences provided herein. Functional or active portions of the RATL1 d6 polypeptide are preferred. The RATLI d6 polypeptide has similarity to ubiquitin conjugating enzymes.

Nucleic acid encoding human RATL1d6 according to the present invention were first identified by subtraction cloning. (Example 1). Additional nucleic acids were identified in Incyte Clone Nos. 2396483 (THP-1, promonocyte library); 5818240 (prostate tumor library); 5396270 (liver tumor library); and 4741202 (thymus library) through BLAST searches of the Incyte database. The sequences were aligned and the resulting contig was utilized to design oligonucleotides to obtain a full-length clone. (See Examples 1 and 2).

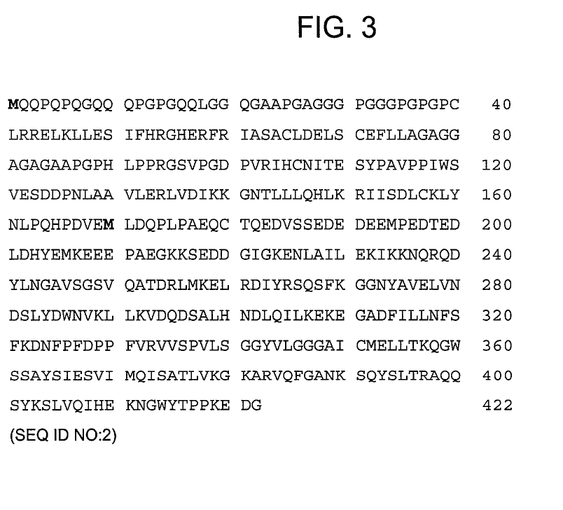

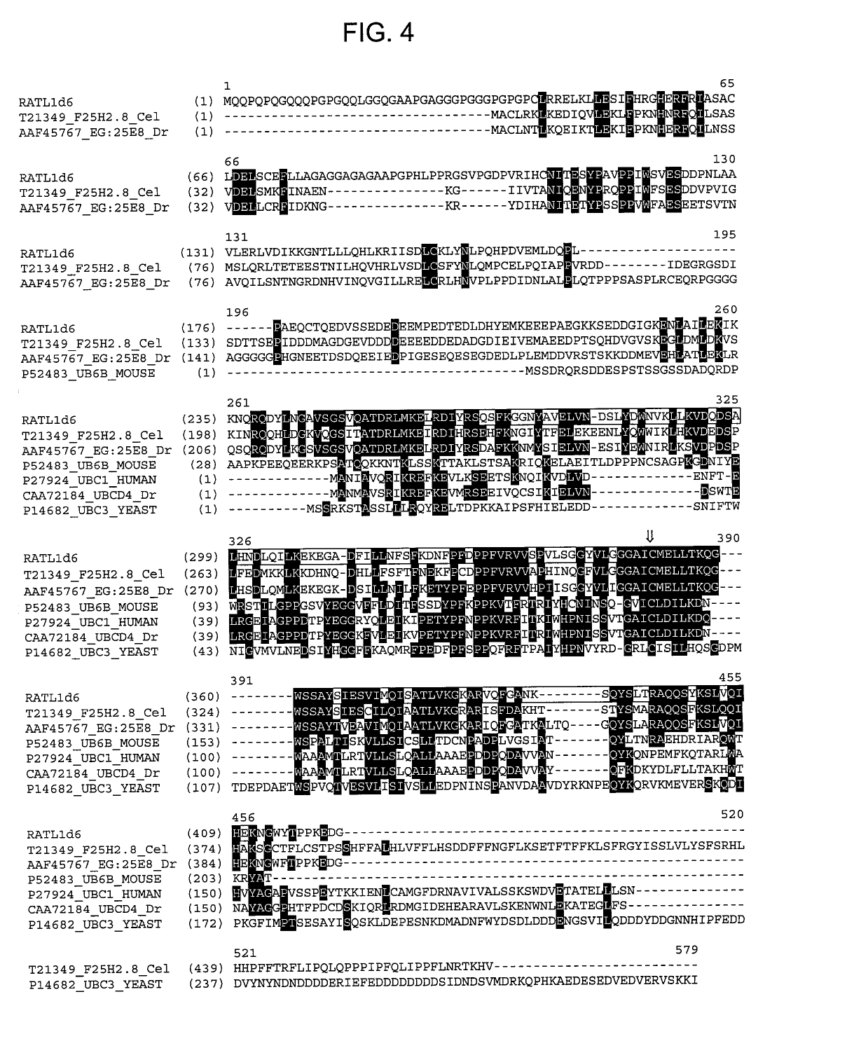

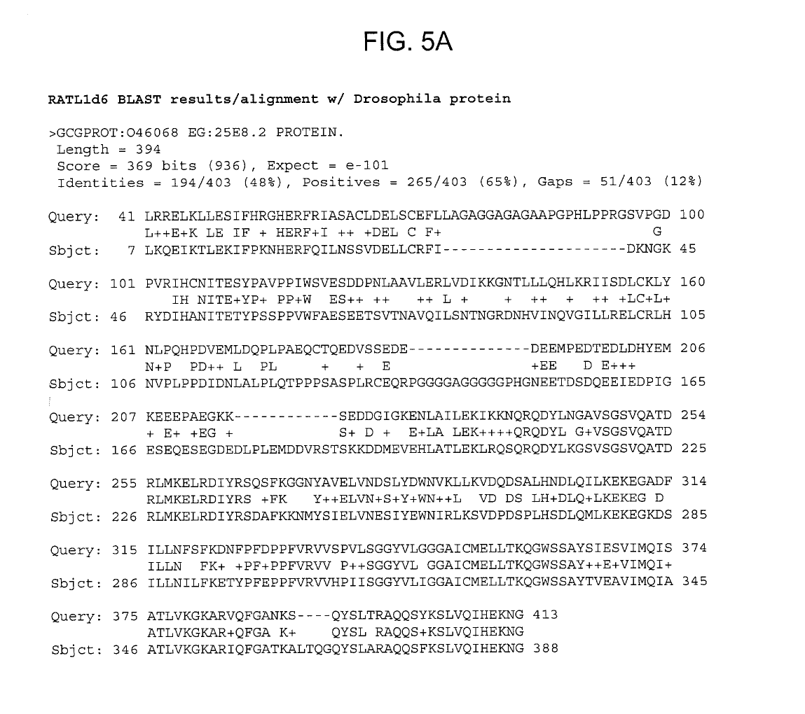

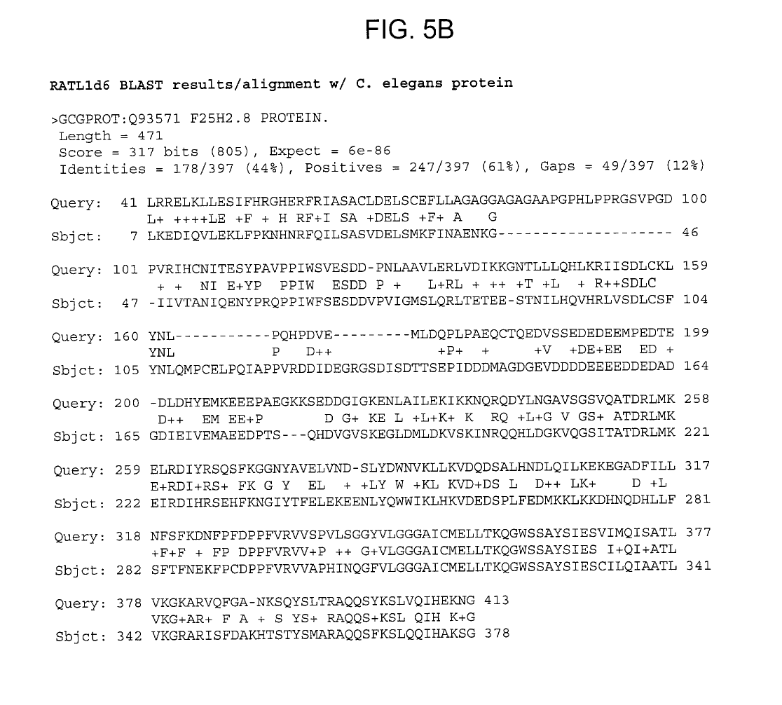

In one of its embodiments, the present invention encompasses a polypeptide comprising the amino acid sequence of SEQ ID NO:2 as shown in FIG. 3. The RAT1d6 polypeptide is 422 amino acids in length and shares amino acid sequence homology to ubiquitin conjugating enzymes. The RATL1 d6 protein is thus characterized as a newly-discovered member of the UBC family isolated from activated T lymphocytes. In addition, FIG. 4 provides an alignment of the RATL1d6 polypeptide sequence with interspecies sequences comprising a ubiquitin conjugating enzyme (UBC) family of proteins having UBC domains.

That EG:25E8.2 is concluded to be a ubiquitin conjugating enzyme and likely ortholog of Rat1d6 is due to the significant level of homology between the proteins. To better elucidate the function of RATL1d6, studies with EG:25E8.2 were undertaken in a Drosophila cell-based model for immunity. (see Example 14).

Mammals have a complex immune response that relies on innate and adaptive immune pathways, and these pathways share similar classes of molecules. Most components of innate immunity are evolutionarily conserved from Drosophila to man, while only higher eukaryotes have acquired immunity (Silverman, N. and Maniatis, T., 2001, Genes Dev., 15:2321-2342).

Insects have a potent and rapid response to a broad spectrum of pathogens. Fungal and bacterial infections of Drosophila lead to transcriptional activation of antimicrobial peptide (AMP) genes. The induction of each AMP gene is regulated by a balance of inputs that are manifested by combinations of the three Rel/NF-κB proteins, namely, Relish, Dorsal and Dif. The AMP AttacinD gene is regulated by activation of Relish homodimers, or heterodimers of Relish and Dorsal (Han, Z. S. and Ip, Y. T., 1999, J. Biol. Chem., 274:21355-21361). Activation of Rel/NF-κB pathways are essential for the Drosophila innate immune response. For example, Drosophila mutations in the Relish gene do not express certain classes of antimicrobial peptides and are susceptible to gram-negative bacterial infection (Hendengren, M. et al., 1999, Mol. Cell., 4(5):827-837).

Drosophila Rel proteins, like mammalian Rels, are sequestered in the cytoplasm as a result of association with an IκB-like inhibitor protein. When cells are activated by pathogens, signaling pathways are activated leading to the degradation of IκB, nuclear translocation of Rel proteins and Rel activated transcription (Silverman, N. and Maniatis, T., 2001, Genes Dev., 15:2321-2342). Cactus is the IκB protein that inhibits Dorsal and Dif. Relish is the mammalian homolog of p105 NF-κB, and like NF-κ-B, Relish contains both a Rel domain and a IκB inhibitory domain. Relish is activated by a cleavage event that releases the IκB domain (Stoven, S. et al., 2000, EMBO Rep., 1:347-352).

The above-discussed studies correlate the function of EG:25E8.2 protein to regulation of the Drosophila innate immune response. Central to these studies was the generation of a “knock out” phenotype with double-stranded RNA-mediated interference (RNAi) of EG:25E8.2 mRNA in Drosophila Schneider 2 (S2) cultured cells. RNAi technologies were developed to produce sustained post-transcriptional gene-silencing and have been reported to work in S2 cells (Caplen, N. J. et al., 2000, Gene, 252:95-105 and Clemens, J. C. et al., 2000, Proc. Natl. Acad. Sci. USA, 97:6499-6503). S2 cells can be induced by the bacterial cell wall component lipopolysaccharide (LPS) to express a subset of antimicrobial peptides, including attacin (Han, Z. S. and Ip, Y. T., 1999, J. Biol. Chem., 274:21355-21361). The experiments described in Example 14 have tested EG:25E8.2 RNAi in a LPS-inducible luciferase reporter system in S2 cells.

Variants of the RATL1d6 polypeptide are also encompassed by the present invention. A preferred RATL1d6 variant has at least 75 to 80%, more preferably at least 85 to 90%, and even more preferably at least 90% amino acid sequence identity to the amino acid sequence claimed herein, and which retains at least one biological, immunological, or other functional characteristic or activity of RATL1 d6 polypeptide. Most preferred is a variant having at least 95% amino acid sequence identity to that of SEQ ID NO:2.

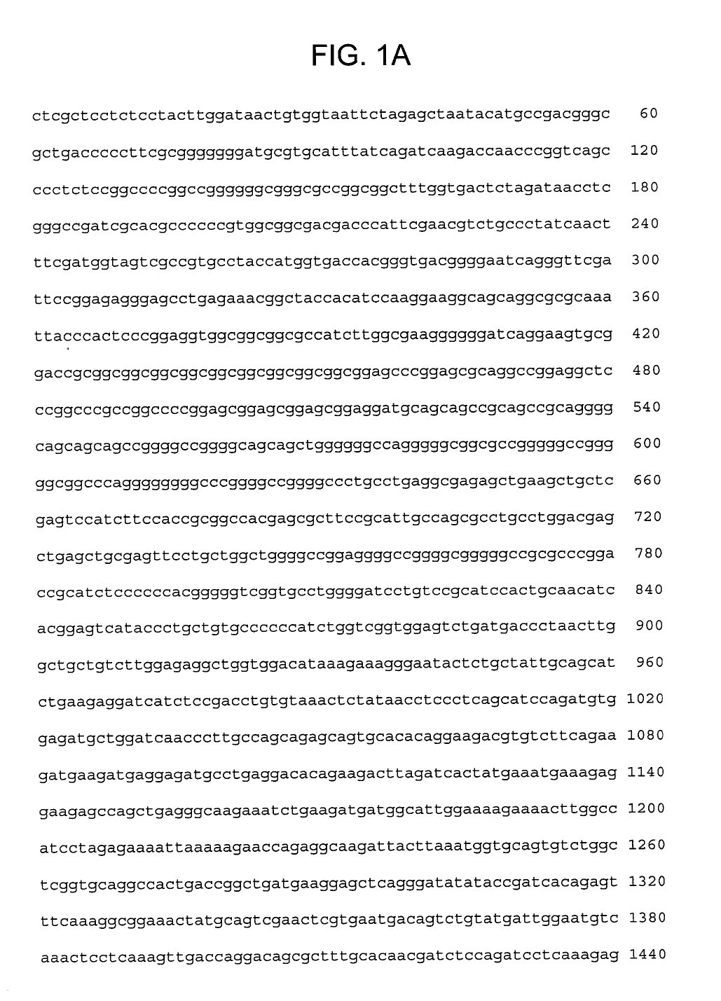

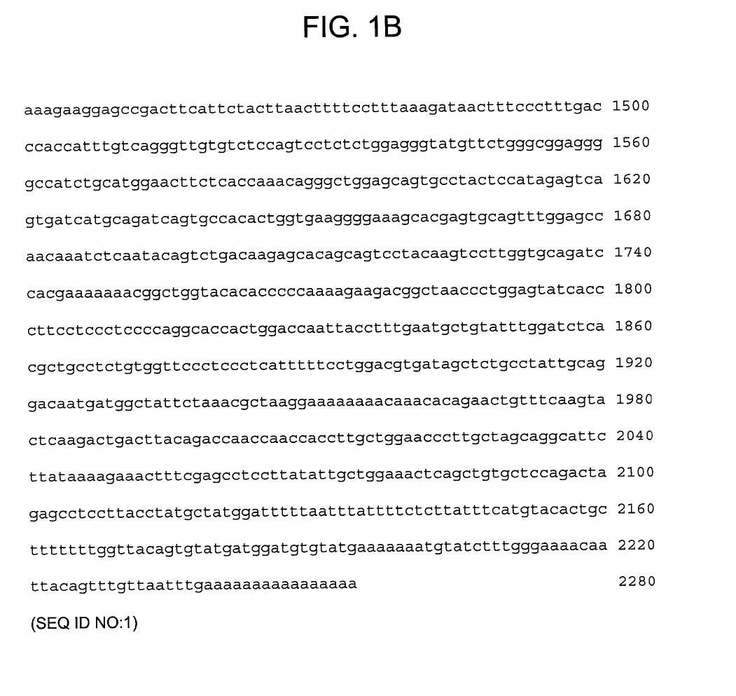

In another embodiment, the present invention encompasses polynucleotides which encode RATL1d6 polypeptide. Accordingly, any nucleic acid sequence which encodes the amino acid sequence of RATL1d6 polypeptide can be used to produce recombinant molecules that express RATL1d6 protein. In a particular embodiment, the present invention encompasses the RATL1 d6 polynucleotide comprising the nucleic acid sequence of SEQ ID NO:1 and as shown in FIGS. 1A and 1B. More particularly, the present invention provides the RATL1d6 clone, deposited at the American Type Culture Collection (ATCC), 10801 University Boulevard, Manassas, Va. 20110-2209 on Oct. 1, 2001 and under ATCC Accession No. PTA-3745 according to the terms of the Budapest Treaty.

As will be appreciated by the skilled practitioner in the art, the degeneracy of the genetic code results in the production of a multitude of nucleotide sequences encoding RATL1d6 polypeptide. Some of the sequences bear minimal homology to the nucleotide sequences of any known and naturally occurring gene. Accordingly, the present invention contemplates each and every possible variation of nucleotide sequence that could be made by selecting combinations based on possible codon choices. These combinations are made in accordance with the standard triplet genetic code as applied to the nucleotide sequence of naturally occurring RATL1d6, and all such variations are to be considered as being specifically disclosed.

Although nucleotide sequences which encode RATL1d6 polypeptide and its variants are preferably capable of hybridizing to the nucleotide sequence of the naturally occurring RATL1d6 polypeptide under appropriately selected conditions of stringency, it may be advantageous to produce nucleotide sequences encoding RATL1d6 polypeptide, or its derivatives, which possess a substantially different codon usage. Codons may be selected to increase the rate at which expression of the peptide/polypeptide occurs in a particular prokaryotic or eukaryotic host in accordance with the frequency with which particular codons are utilized by the host. Other reasons for substantially altering the nucleotide sequence encoding RATL1d6 polypeptide, and its derivatives, without altering the encoded amino acid sequences include the production of RNA transcripts having more desirable properties, such as a greater half-life, than transcripts produced from the naturally occurring sequence.

The present invention also encompasses production of DNA sequences, or portions thereof, which encode RATL1d6 polypeptide, and its derivatives, entirely by synthetic chemistry. After production, the synthetic sequence may be inserted into any of the many available expression vectors and cell systems using reagents that are well known and practiced by those in the art. Moreover, synthetic chemistry may be used to introduce mutations into a sequence encoding RATL1d6 polypeptide, or any fragment thereof.

Also encompassed by the present invention are polynucleotide sequences that are capable of hybridizing to the claimed nucleotide sequence of RATL1d6, such as that shown in SEQ ID NO:1, under various conditions of stringency. Hybridization conditions are typically based on the melting temperature (Tₘ) of the nucleic acid binding complex or probe (See, G. M. Wahl and S. L. Berger, 1987; Methods Enzymol., 152:399-407 and A. R. Kimmel, 1987; Methods of Enzymol., 152:507-511), and may be used at a defined stringency. For example, included in the present invention are sequences capable of hybridizing under moderately stringent conditions to the RATL1d6 sequence of SEQ ID NO:1 and other sequences which are degenerate to those which encode RATL1d6 polypeptide (e.g., as a nonlimiting example: prewashing solution of 2×SSC, 0.5% SDS, 1.0 mM EDTA, pH 8.0, and hybridization conditions of 50° C., 5×SSC, overnight.

The nucleic acid sequence encoding RATL1d6 protein may be extended utilizing a partial nucleotide sequence and employing various methods known in the art to detect upstream sequences such as promoters and regulatory elements. For example, one method which may be employed is restriction-site PCR, which utilizes universal primers to retrieve unknown sequence adjacent to a known locus (G. Sarkar, 1993, PCR Methods Applic., 2:318-322). In particular, genomic DNA is first amplified in the presence of primer to a linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase.

Inverse PCR may also be used to amplify or extend sequences using divergent primers based on a known region or sequence (T. Triglia et al., 1988, Nucleic Acids Res., 16:8186). The primers may be designed using OLIGO 4.06 Primer Analysis software (National Biosciences Inc., Plymouth, Minn.), or another appropriate program, to be 22-30 nucleotides in length, to have a GC content of 50% or more, and to anneal to the target sequence at temperatures about 68⁰-72° C. The method uses several restriction enzymes to generate a suitable fragment in the known region of a gene. The fragment is then circularized by intramolecular ligation and used as a PCR template.

Another method which may be used is capture PCR which involves PCR amplification of DNA fragments adjacent to a known sequence in human and yeast artificial chromosome (YAC) DNA (M. Lagerstrom et al., 1991, PCR Methods Applic., 1:111-119). In this method, multiple restriction enzyme digestions and ligations may also be used to place an engineered double-stranded sequence into an unknown portion of the DNA molecule before performing PCR. J. D. Parker et al. (1991; Nucleic Acids Res., 19:3055-3060) provide another method which may be used to retrieve unknown sequences. In addition, PCR, nested primers, and PROMOTERFINDER libraries can be used to walk genomic DNA (Clontech, Palo Alto, Calif.). This process avoids the need to screen libraries and is useful in finding intron/exon junctions.

When screening for full-length cDNAs, it is preferable to use libraries that have been size-selected to include larger cDNAs. Also, random-primed libraries are preferable, since they will contain more sequences which contain the 5′ regions of genes. The use of a randomly primed library may be especially preferable for situations in which an oligo d(T) library does not yield a full-length cDNA. Genomic libraries may be useful for extension of sequence into the 5′ and 3′ non-transcribed regulatory regions, or to identify exon usage in alternatively-spliced transcripts.

The embodiments of the present invention can be practiced using methods for DNA sequencing which are well known and generally available in the art. The methods may employ such enzymes as the Klenow fragment of DNA polymerase 1, SEQUENASE (US Biochemical Corp. Cleveland, Ohio), Taq polymerase (PE Biosystems), thermostable T7 polymerase (Amersham Pharmacia Biotech, Piscataway, N.J.), or combinations of recombinant polymerases and proofreading exonucleases such as the ELONGASE Amplification System marketed by Life Technologies (Gaithersburg, Md.). Preferably, the process is automated with machines such as the Hamilton Micro Lab 2200 (Hamilton, Reno, Nev.), Peltier Thermal Cycler (PTC200; MJ Research, Watertown, Mass.) and the ABI Catalyst and 373 and 377 DNA sequencers (PE Biosystems).

Commercially available capillary electrophoresis systems may be used to analyze the size or confirm the nucleotide sequence of sequencing or PCR products. In particular, capillary sequencing may employ flowable polymers for electrophoretic separation, four different fluorescent dyes (one for each nucleotide) which are laser activated, and detection of the emitted wavelengths by a charge coupled device camera. Output/light intensity may be converted to electrical signal using appropriate software (e.g., GENOTYPER and SEQUENCE NAVIGATOR, PE Biosystems) and the entire process—from loading of samples to computer analysis and electronic data display—may be computer controlled. Capillary electrophoresis is especially preferable for the sequencing of small pieces of DNA which might be present in limited amounts in a particular sample.

In another embodiment of the present invention, polynucleotide sequences or fragments thereof which encode RATL1d6 polypeptide, or peptides thereof, may be used in recombinant DNA molecules to direct the expression of RATL1d6 polypeptide product, or fragments or functional equivalents thereof, in appropriate host cells. Because of the inherent degeneracy of the genetic code, other DNA sequences, which encode substantially the same or a functionally equivalent amino acid sequence, may be produced and these sequences may be used to clone and express RATL1d6 protein.

As will be appreciated by those having skill in the art, it may be advantageous to produce RATL1d6 polypeptide-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence.

The nucleotide sequence of the present invention can be engineered using methods generally known in the art in order to alter RATL1d6 polypeptide-encoding sequences for a variety of reasons, including, but not limited to, alterations which modify the cloning, processing, and/or expression of the gene product. DNA shuffling by random fragmentation and PCR reassembly of gene fragments and synthetic oligonucleotides may be used to engineer the nucleotide sequences. For example, site-directed (or site-specific) mutagenesis may be used to insert new restriction sites, alter glycosylation patterns, change codon preference, produce splice variants, or introduce mutations, and the like. A variety of techniques for performing site-directed mutagenesis are known and practiced in the art and are described, for example, in M. J. McPherson (ed), 1991, Directed Mutagenesis: A Practical Approach, IRL Press, Oxford; J. L. Owen et al., Apr., 1994, Focus, Life Technologies, Inc., Vol. 16.2:39-44; and R. Andag and E. Schutz, 2001, BioTechniques, 30(3):486-488). Kits for performing in vitro site-directed mutagenesis are also commercially available and widely used (e.g., Quik-Change® Site-Directed Mutagenesis Kit, Stratagene, La Jolla, Calif.; and Unique Site Elimination Mutagenesis Kit, Pharmacia Biotechnology, Piscataway, N.J.).

In another embodiment of the present invention, natural, modified, or recombinant nucleic acid sequences encoding RATL1d6 polypeptide may be ligated to a heterologous sequence to encode a fusion protein. For example, for screening peptide libraries for inhibitors of RATL1d6 activity, it may be useful to encode a chimeric RATL1d6 protein that can be recognized by a commercially available antibody. A fusion protein may also be engineered to contain a cleavage site located between the RATL1d6 protein-encoding sequence and the heterologous protein sequence, so that RATL1d6 protein may be cleaved and purified away from the heterologous moiety.

In another embodiment, ligand-binding assays are useful to identify inhibitor compounds that interfere with the function of the RATL1d6 product. Such assays are useful even if the function of a protein is not known. These assays are designed to detect binding of test compounds to particular target molecules, e.g., proteins or peptides. The detection may involve direct measurement of binding. Alternatively, indirect indications of binding may involve stabilization of protein structure or disruption of a biological function. Non-limiting examples of useful ligand-binding assays are detailed below.

One useful method for the detection and isolation of binding proteins is the Biomolecular Interaction Assay (BIAcore) system developed by Pharmacia Biosensor and described in the manufacturer's protocol (LKB Pharmacia, Sweden). The BIAcore system uses an affinity purified anti-GST antibody to immobilize GST-fusion proteins onto a sensor chip. The sensor utilizes surface plasmon resonance, which is an optical phenomenon that detects changes in refractive indices. Accordingly, a protein of interest, e.g., the RATL1d6 polypeptide, or fragment thereof, of the present invention, is coated onto a chip and test compounds are passed over the chip. Binding is detected by a change in the refractive index (surface plasmon resonance).

A different type of ligand-binding assay involves scintillation proximity assays (SPA), as described in U.S. Pat. No. 4,568,649. In a modification of this assay currently undergoing development, chaperoning are used to distinguish folded and unfolded proteins. A tagged protein is attached to SPA beads, and test compounds are added. The bead is then subjected to mild denaturing conditions, such as, for example, heat, exposure to SDS, and the like, and a purified labeled chaperonin is added. If a test compound has bound to a target protein, the labeled chaperonin will not bind; conversely, if no test compound has bound, the protein will undergo some degree of denaturation and the chaperonin will bind. In another type of ligand binding assay, proteins containing mitochondrial targeting signals are imported into isolated mitochondria in vitro (Hurt et al., 1985, EMBO J., 4:2061-2068; Eilers and Schatz, 1986, Nature, 322:228-231). In a mitochondrial import assay, expression vectors are constructed in which nucleic acids encoding particular target proteins are inserted downstream of sequences encoding mitochondrial import signals. The chimeric proteins are synthesized and tested for their ability to be imported into isolated mitochondria in the absence and presence of test compounds. A test compound that binds to the target protein should inhibit its uptake into isolated mitochondria in vitro.

Another type of ligand-binding assay suitable for use according to the present invention is the yeast two-hybrid system (Fields and Song, 1989, Nature, 340:245-246). The yeast two-hybrid system takes advantage of the properties of the GAL4 protein of the yeast S. cerevisiae. The GAL4 protein is a transcriptional activator required for the expression of genes encoding enzymes involving the utilization of galactose. GAL4 protein consists of two separable and functionally essential domains: an N-terminal domain, which binds to specific DNA sequences (UASG); and a C-terminal domain containing acidic regions, which is necessary to activate transcription. The native GAL4 protein, containing both domains, is a potent activator of transcription when yeast cells are grown on galactose medium. The N-terminal domain binds to DNA in a sequence-specific manner but is unable to activate transcription. The C-terminal domain contains the activating regions but cannot activate transcription because it fails to be localized to UASG. In the two-hybrid system, a system of two hybrid proteins containing parts of GAL4: (1) a GAL4 DNA-binding domain fused to a protein ‘X’, and (2) a GAL4 activation region fused to a protein ‘Y’. If X and Y can form a protein-protein complex and reconstitute proximity of the GAL4 domains, transcription of a gene regulated by UASG occurs. Creation of two hybrid proteins, each containing one of the interacting proteins X and Y, allows the activation region of UASG to be brought to its normal site of action.

The binding assay described in Fodor et al., 1991, Science, 251:767-773, which involves testing the binding affinity of test compounds for a plurality of defined polymers synthesized on a solid substrate, may also be useful. Compounds that bind to the RATL1d6 polypeptide, or portions thereof, according to this invention are potentially useful as agents for use in therapeutic compositions.

In another embodiment, sequences encoding the RATL1d6 polypeptide may be synthesized in whole, or in part, using chemical methods well known in the art (See, for example, M. H. Caruthers et al., 1980, Nucl. Acids Res. Symp. Ser., 215-223 and T. Horn, T et al., 1980, Nucl. Acids Res. Symp. Ser., 225-232). Alternatively, the protein itself may be produced using chemical methods to synthesize the amino acid sequence of RATL1d6 polypeptide, or a fragment or portion thereof. For example, peptide synthesis can be performed using various solid-phase techniques (J. Y. Roberge et al., 1995, Science, 269:202-204) and automated synthesis may be achieved, for example, using the ABI 431A Peptide Synthesizer (PE Biosystems). The newly synthesized peptide can be substantially purified by preparative high performance liquid chromatography (e.g., T. Creighton, 1983, Proteins, Structures and Molecular Principles, W H Freeman and Co., New York, N.Y), by reversed-phase high performance liquid chromatography, or other purification methods as are known in the art. The composition of the synthetic peptides may be confirmed by amino acid analysis or sequencing (e.g., the Edman degradation procedure; Creighton, supra). In addition, the amino acid sequence of RATL1d6 polypeptide or any portion thereof, may be altered during direct synthesis and/or combined using chemical methods with sequences from other proteins, or any part thereof, to produce a variant polypeptide.

Polypeptide Lacking a Start Methionine

In a preferred embodiment, the present invention encompasses a polynucleotide lacking the initiating start codon, in addition to the resulting encoded polypeptide of RATL1d6. Specifically, the present invention encompasses the polynucleotide corresponding to nucleotides 520 through 1782 of SEQ ID NO:1, and the polypeptide corresponding to amino acids 2 through 422 of SEQ ID NO:2. Also encompassed by this invention are recombinant vectors comprising the polynucleotide sequence encoding RATL1 d6, and host cells comprising the vector. RATL1d6 UBC Domain

The UBC domain of the novel RATL1d6 polypeptide is located from about G248 to about K411 of SEQ ID NO:2. The conserved cysteine involved in ubiquitin transfer is located at amino acid 351 of SEQ ID NO:2.

In a preferred embodiment, the following RATL1d6 UBC domain polypeptide is encompassed by the present invention: GSVQATDRLMKELRDIYRSQSFKGGNYAVELVNDSLYDWNVKLLKVDQD SALHNDLQILKEKEGADFILLNFSFKDNFPFDPPFVRVVSPVLSGGYVLGG GAICMELLTKQGWSSAYSIESVIMQISATLVKGKARVQFGANKSQYSLTRA QQSYKSLVQIHEK (SEQ ID NO:47). The polynucleotide encoding this polypeptide is also provided. The present invention also encompasses the use of the RATL1d6 UBC domain polypeptide as an immunogenic and/or antigenic epitope as described elsewhere herein.

In additional preferred embodiments, the following N-terminal RATL1d6 UBC domain deletion polypeptides are encompassed by the present invention: G1-K164, S2-K164, V3-K164, Q4-K164, A5-K164, T6-K164, D7-K164, R8-K164, L9-K164, M10-K164, K11-K164, E12-K164, L13-K164, R14-K164, D15-K164, I16-K164, Y17-K164, R18-K164, S19-K164, Q20-K164, S21-K164, F22-K164, K23-K164, G24-K164, G25-K164, N26-K164, Y27-K164, A28-K164, V29-K164, E30-K164, L31-K164, V32-K164, N33-K164, D34-K164, S35-K164, L36-K164, Y37-K164, D38-K164, W39-K164, N40-K164, V41-K164, K42-K164, L43-K164, L44-K164, K45-K164, V46-K164, D47-K164, Q48-K164, D49-K164, S50-K164, A51-K164, L52-K164, H53-K164, N54-K164, D55-K164, L56-K164, Q57-K164, I58-K164, L59-K164, K60-K164, E61-K164, K62-K164, E63-K164, G64-K164, A65-K164, D66-K164, F67-K164, I68-K164, L69-K164, L70-K164, N71-K164, F72-K164, S73-K164, F74-K164, K75-K164, D76-K164, N77-K164, F78-K164, P79-K164, F80-K164, D81-K164, P82-K164, P83-K164, F84-K164, V85-K164, R86-K164, V87-K164, V88-K164, S89-K164, P90-K164, V91-K164, L92-K164, S93-K164, G94-K164, G95-K164, Y96-K164, V97-K164, L98-K164, G99-K164, G100-K164, G101-K164, A102-K164, 1103-K164, C104-K164, M105-K164, E106-K164, L107-K164, L108-K164, T109-K164, K110-K164, Q111-K164, G112-K164, W113-K164, S114-K164, S115-K164, A116-K164, Y117-K164, S 118-K164, 1119-K164, E120-K164, S121-K164, V122-K164, 1123-K164, M124-K164, Q125-K164, 1126-K164, S127-K164, A128-K164, T129-K164, L130-K164, V131-K164, K132-K164, G133-K164, K134-K164, A135-K164, R136-K164, V137-K164, Q138-K164, F139-K164, G140-K164, A141-K164, N142-K164, K143-K164, S144-K164, Q145-K164, Y146-K164, S147-K164, L148-K164, T149-K164, R150-K164, A151-K164, Q152-K164, Q153-K164, S154-K164, Y155-K164, K156-K164, S157-K164, and/or L158-K164 of SEQ ID NO:2. Polynucleotide sequences encoding these polypeptides are also provided. The present invention also encompasses the use of one or more of these N-terminal RATL1 d6 UBC domain deletion polypeptides as immunogenic and/or antigenic epitopes as described elsewhere herein.

In yet other preferred embodiments, the following C-terminal RATL1d6 UBC domain deletion polypeptides are encompassed by the present invention: G1-K164, G1-E163, G1-H162, GI-1161, G1-Q160, G1-V159, G1-L158, G1-S157, G1-K156, G1-Y155, G1-S154, G1-Q153, G1-Q152, G1-A151, G1-R150, G1-T149, G1-L148, G1-S147, G1-Y146, G1-Q145, G1-S144, G1-K143, G1-N142, G1-A141, G1-G140, G1-F139, G1-Q138, G1-V137, G1-R136, G1-A135, G1-K134, G1-G133, G1-K132, G1-V131, G1-L130, G1-T129, G1-A128, G1-S127, G1-1126, G1-Q125, G1-M124, G1-1123, G1-V122, G1-S121, G1-E120, G1-1119, G1-S118, G1-Y117, G1-Al 16, G1-S115, G1-S114, G1-W113, G1-I112, G1-Q111, G1-K110, G1-T109, G1-L108, G1-L107, G1-E106, G1-M105, G1-C104, G1-I103, G1-A102, G1-G101, G1-G100, G1-G99, G1-L98, G1-V97, G1-Y96, G1-G95, G1-G94, G1-S93, G1-L92, G1-V91, G1-P90, G1-S89, G1-V88, G1-V87, G1-R86, G1-V85, G1-F84, G1-P83, G1-P82, G1-D81, G1-F80, G1-P79, G1-F78, G1-N77, G1-D76, G1-K75, G1-F74, G1-S73, G1-F72, G1-N71, G1-L70, G1-L69, G1-168, G1-F67, G1-D66, G1-A65, G1-G64, G1-E63, G1-K62, G1-E61, G1-K60, G1-L59, G1-158, G1-Q57, G1-L56, G1-D55, G1-N54, G1-H53, G1-L52, G1-A51, G1-S50, G1-D49, G1-Q48, G1-D47, G1-V46, G1-K45, G1-L44, G1-L43, G1-K42, G1-V41, G1-N40, G1-W39, G1-D38, G1-Y37, G1-L36, G1-S35, G1-D34, G1-N33, G1-V32, G1-L31, G1-E30, G1-V29, G1-A28, G1-Y27, G1-N26, G1-G25, G1-G24, G1-K23, G1-F22, G1-S21, G1-Q20, G1-S19, G1-R18, G1-Y17, G1-116, G1-D15, G1-R14, G1-L13, G1-E12, G1-K11, G1-M10, G1-L9, G1-R8, and/or G1-D7 of SEQ ID NO:2. Polynucleotide sequences encoding these polypeptides are also provided. The present invention also encompasses the use of one or more of these C-terminal RATL1d6 UBC domain deletion polypeptides as immunogenic and/or antigenic epitopes as described elsewhere herein.

In additional preferred embodiments, the following RATL1d6 UBC domain amino acid substitutions are encompassed by the present invention: wherein G248 is substituted with either an A, C, D, E, F, H, I, K, L, M, N, P, Q, R, S, T, V, W, or Y amino acid residue; wherein S249 is substituted with either an A, C, D, E, F, G, H, I, K, L, M, N, P, Q, R, T, V, W, or Y amino acid residue; wherein V250 is substituted with either an A, C, D, E, F, G, H, I, K, L, M, N, P, Q, R, S, T, W, or Y amino acid residue; wherein Q251 is substituted with either an A, C, D, E, F, G, H, I, K, L, M, N, P, R, S, T, V, W, or Y amino acid residue; wherein A252 is substituted with either a C, D, E, F, G, H, I, K, L, M, N, P, Q, R, S, T, V, W, or Y amino acid residue; wherein T253 is substituted with either an A, C, D, E, F, G, H, I, K, L, M, N, P, Q, R, S, V, W, or Y amino acid residue; wherein D254 is substituted with either an A, C, E, F, G, H, I, K, L, M, N, P, Q, R, S, T, V, W, or Y amino acid residue; wherein R255 is substituted with either an A, C, D, E, F, G, H, I, K, L, M, N, P, Q, S, T, V, W, or Y amino acid residue; wherein L256 is substituted with either an A, C, D, E, F, G, H, I, K, M, N, P, Q, R, S, T, V, W, or Y amino acid residue; wherein M257 is substituted with either an A, C, D, E, F, G, H, I, K, L, N, P, Q, R, S, T, V, W, or Y amino acid residue; wherein K258 is substituted with either an A, C, D, E, F, G, H, I, L, M, N, P, Q, R, S, T, V, W, or Y amino acid residue; wherein E259 is substituted with either an A, C, D, F, G, H, I, K, L, M, N, P, Q, R, S, T, V, W, or Y amino acid residue; wherein L260 is substituted with either an A, C, D, E, F, G, H, I, K, M, N, P, Q, R, S, T, V, W, or Y amino acid residue; wherein R261 is substituted with either an A, C, D, E, F, G, H, I, K, L, M, N, P, Q, S, T, V, W, or Y amino acid residue; wherein D262 is substituted with either an A, C, E, F, G, H, I, K, L, M, N, P, Q, R, S, T, V, W, or Y amino acid residue; wherein 1263 is substituted with either an A, C, D, E, F, G, H, K, L, M, N, P, Q, R, S, T, V, W, or Y amino acid residue; wherein Y264 is substituted with either an A, C, D, E, F, G, H, I, K, L, M, N, P, O, R, S, T, V, or W amino acid residue; wherein R265 is substituted with either an A, C, D, E, F, G, H, I, K, L, M, N, P, Q, S, T V, W, or Y amino acid residue; wherein S266 is substituted with either an A, C, D, E, F, G, H, I, K, L, M, N, P, Q, R, T, V, W, or Y amino acid residue; wherein Q267 is substituted with either an A, C, D, E, F, G, H, I, K, L, M, N, P, R, S, T, V, W, or Y amino acid residue; wherein S268 is substituted with either an A, C, D, E, F, G, H, I, K, L, M, N, P, Q, R, T, V, W, or Y amino acid residue; wherein F269 is substituted with either an A, C, D, E, G, H, I, K, L, M, N, P, Q, R, S, T, V, W, or Y amino acid residue; wherein K270 is substituted with either an A, C, D, E, F, G, H, I, L, M, N, P, Q, R, S, T, V, W, or Y amino acid residue; wherein G271 is substituted with either an A, C, D, E, F, H, I, K, L, M, N, P, Q, R, S, T, V, W, or Y amino acid residue; wherein G272 is substituted with either an A, C, D, E, F, H, I, K, L, M, N, P, Q, R, S, T, V, W, or Y amino acid residue; wherein N273 is substituted with either an A, C, D, E, F, G, H, I, K, L, M, P, Q, R, S, T, V, W, or Y amino acid residue; wherein Y274 is substituted with either an A, C, D, E, F, G, H, I, K, L, M, N, P, Q, R, S, T, V, or W amino acid residue; wherein A275 is substituted with either a C, D, E, F, G, H, I, K, L, M, N, P, Q, R, S, T, V, W, or Y amino acid residue; wherein V276 is substituted with either an A, C, D, E, F, G, H, I, K, L, M, N, P, Q, R, S, T, W, or Y amino acid residue; wherein E277 is substituted with either an A, C, D, F, G, H, I, K, L, M, N, P, Q, R, S, T, V, W, or Y amino acid residue; wherein L278 is substituted with either an A, C, D, E, F, G, H, I, K, M, N, P, Q, R, S, T, V, W, or Y amino acid residue; wherein V279 is substituted with either an A, C, D, E, F, G, H, I, K, L, M, N, P, Q, R, S, T, W, or Y amino acid residue; wherein N280 is substituted with either an A, C, D, E, F, G, H, I, K, L, M, P, Q, R, S, T, V, W, or Y amino acid residue; wherein D281 is substituted with either an A, C, E, F, G, H, I, K, L, M, N, P, Q, R, S, T, V, W, or Y amino acid residue; wherein S282 is substituted with either an A, C, D, E, F, G, H, I, K, L, M, N, P, Q, R, T, V, W, or Y amino acid residue; wherein L283 is substituted with either an A, C, D, E, F, G, H, I, K, M, N, P, Q, R, S, T, V, W, or Y amino acid residue; wherein Y284 is substituted with either an A, C, D, E, F, G, H, I, K, L, M, N, P, Q, R, S, T, V, or W amino acid residue; wherein D285 is substituted with either an A, C, E, F, G, H, I, K, L, M, N, P, Q, R, S, T, V, W, or Y amino acid residue; wherein W286 is substituted with either an A, C, D, E, F, G, H, I, K, L, M, N, P, Q, R, S, T, V, or Y amino acid residue; wherein N287 is substituted with either an A, C, D, E, F, G, H, I, K, L, M, P, Q, R, S, T, V, W, or Y amino acid residue; wherein V288 is substituted with either an A, C, D, E, F, G, H, I, K, L, M, N, P, Q, R, S, T, W, or Y amino acid residue; wherein K289 is substituted with either an A, C, D, E, F, G, H, I, L, M, N, P, Q, R, S, T, V, W, or Y amino acid residue; wherein L290 is substituted with either an A, C, D, E, F, G, H, I, K, M, N, P, Q, R, S, T, V, W, or Y amino acid residue; wherein L291 is substituted with either an A, C, D, E, F, G, H, I, K, M, N, P, Q, R, S, T, V, W, or Y amino acid residue; wherein K292 is substituted with either an A, C, D, E, F, G, H, I, L, M, N, P, Q, R, S, T, V, W, or Y amino acid residue; wherein V293 is substituted with either an A, C, D, E, F, G, H, I, K, L, M, N, P, Q, R, S, T, W, or Y amino acid residue; wherein D294 is substituted with either an A, C, E, F, G, H, I, K, L, M, N, P, Q, R, S, T, V, W, or Y amino acid residue; wherein Q295 is substituted with either an A, C, D, E, F, G, H, I, K, L, M, N, P, R, S, T, V, W, or Y amino acid residue; wherein D296 is substituted with either an A, C, E, F, G, H, I, K, L, M, N, P, Q, R, S, T, V, W, or Y amino acid residue; wherein S297 is substituted with either an A, C, D, E, F, G, H, I, K, L, M, N, P, Q, R, T, V, W, or Y amino acid residue; wherein A298 is substituted with either a C, D, E, F, G, H, I, K, L, M, N, P, Q, R, S, T, V, W, or Y amino acid residue; wherein L299 is substituted with either an A, C, D, E, F, G, H, I, K, M, N, P, Q, R, S, T, V, W, or Y amino acid residue; wherein H300 is substituted with either an A, C, D, E, F, G, I, K, L, M, N, P, Q, R, S, T, V, W, or Y amino acid residue; wherein N301 is substituted with either an A, C, D, E, F, G, H, I, K, L, M, P, Q, R, S, T, V, W, or Y amino acid residue; wherein D302 is substituted with either an A, C, E, F, G, H, I, K, L, M, N, P, Q, R, S, T, V, W, or Y amino acid residue; wherein L303 is substituted with either an A, C, D, E, F, G, H, I, K, M, N, P, Q, R, S, T, V, W, or Y amino acid residue; wherein Q304 is substituted with either an A, C, D, E, F, G, H, I, K, L, M, N, P, R, S, T, V, W, or Y amino acid residue; wherein 1305 is substituted with either an A, C, D, E, F, G, H, K, L, M, N, P, Q, R, S, T, V, W, or Y amino acid residue; wherein L306 is substituted with either an A, C, D, E, F, G, H, I, K, M, N, P, Q, R, S, T, V, W, or Y amino acid residue; wherein K307 is substituted with either an A, C, D, E, F, G, H, I, L, M, N, P, Q, R, S, T, V, W, or Y amino acid residue; wherein E308 is substituted with either an A, C, D, F, G, H, I, K, L, M, N, P, O, R, S, T, V, W, or Y amino acid residue; wherein K309 is substituted with either an A, C, D, E, F, G, H, I, L, M, N, P, Q, R, S, T, V, W, or Y amino acid residue; wherein E310 is substituted with either an A, C, D, F, G, H, I, K, L, M, N, P, Q, R, S, T, V, W, or Y amino acid residue; wherein G311 is substituted with either an A, C, D, E, F, H, I, K, L, M, N, P, Q, R, S, T, V, W, or Y amino acid residue; wherein A312 is substituted with either a C, D, E, F, G, H, I, K, L, M, N, P, Q, R, S, T, V, W, or Y amino acid residue; wherein D313 is substituted with either an A, C, E, F, G, H, I, K, L, M, N, P, Q, R, S, T, V, W, or Y amino acid residue; wherein F314 is substituted with either an A, C, D, E, G, H, I, K, L, M, N, P, Q, R, S, T, V, W, or Y amino acid residue; wherein 1315 is substituted with either an A, C, D, E, F, G, H, K, L, M, N, P, Q, R, S, T, V, W, or Y amino acid residue; wherein L316 is substituted with either an A, C, D, E, F, G, H, I, K, M, N, P, Q, R, S, T, V, W, or Y amino acid residue; wherein L317 is substituted with either an A, C, D, E, F, G, H, I, K, M, N, P, Q, R, S, T, V, W, or Y amino acid residue; wherein N318 is substituted with either an A, C, D, E, F, G, H, I, K, L, M, P, Q, R, S, T, V, W, or Y amino acid residue; wherein F319 is substituted with either an A, C, D, E, G, H, I, K, L, M, N, P, Q, R, S, T, V, W, or Y amino acid residue; wherein S320 is substituted with either an A, C, D, E, F, G, H, I, K, L, M, N, P, Q, R, T, V, W, or Y amino acid residue; wherein F321 is substituted with either an A, C, D, E, G, H, I, K, L, M, N, P, Q, R, S, T, V, W, or Y amino acid residue; wherein K322 is substituted with either an A, C, D, E, F, G, H, I, L, M, N, P, Q, R, S, T, V, W, or Y amino acid residue; wherein D323 is substituted with either an A, C, E, F, G, H, I, K, L, M, N, P, Q, R, S, T, V, W, or Y amino acid residue; wherein N324 is substituted with either an A, C, D, E, F, G, H, I, K, L, M, P, Q, R, S, T, V, W, or Y amino acid residue; wherein F325 is substituted with either an A, C, D, E, G, H, I, K, L, M, N, P, Q, R, S, T, V, W, or Y amino acid residue; wherein P326 is substituted with either an A, C, D, E, F, G, H, I, K, L, M, N, Q, R, S, T, V, W, or Y amino acid residue; wherein F327 is substituted with either an A, C, D, E, G, H, I, K, L, M, N, P, Q, R, S, T, V, W, or Y amino acid residue; wherein D328 is substituted with either an A, C, E, F, G, H, I, K, L, M, N, P, Q, R, S, T, V, W, or Y amino acid residue; wherein P329 is substituted with either an A, C, D, E, F, G, H, I, K, L, M, N, Q, R, S, T, V, W, or Y amino acid residue; wherein P330 is substituted with either an A, C, D, E, F, G, H, I, K, L, M, N, Q, R, S, T, V, W, or Y amino acid residue; wherein F331 is substituted with either an A, C, D, E, G, H, I, K, L, M, N, P, Q, R, S, T, V, W, or Y amino acid residue; wherein V332 is substituted with either an A, C, D, E, F, G, H, I, K, L, M, N, P, Q, R, S, T, W, or Y amino acid residue; wherein R333 is substituted with either an A, C, D, E, F, G, H, I, K, L, M, N, P, Q, S, T, V, W, or Y amino acid residue; wherein V334 is substituted with either an A, C, D, E, F, G, H, I, K, L, M, N, P, Q, R, S, T, W, or Y amino acid residue; wherein V335 is substituted with either an A, C, D, E, F, G, H, I, K, L, M, N, P, Q, R, S, T, W, or Y amino acid residue; wherein S336 is substituted with either an A, C, D, E, F, G, H, I, K, L, M, N, P, Q, R, T, V, W, or Y amino acid residue; wherein P337 is substituted with either an A, C, D, E, F, G, H, I, K, L, M, N, Q, R, S, T, V, W, or Y amino acid residue; wherein V338 is substituted with either an A, C, D, E, F, G, H, I, K, L, M, N, P, Q, R, S, T, W, or Y amino acid residue; wherein L339 is substituted with either an A, C, D, E, F, G, H, I, K, M, N, P, Q, R, S, T, V, W, or Y amino acid residue; wherein S340 is substituted with either an A, C, D, E, F, G, H, I, K, L, M, N, P, Q, R, T, V, W, or Y amino acid residue; wherein G341 is substituted with either an A, C, D, E, F, H, I, K, L, M, N, P, Q, R, S, T, V, W, or Y amino acid residue; wherein G342 is substituted with either an A, C, D, E, F, H, I, K, L, M, N, P, Q, R, S, T, V, W, or Y amino acid residue; wherein Y343 is substituted with either an A, C, D, E, F, G, H, I, K, L, M, N, P, Q, R, S, T, V, or W amino acid residue; wherein V344 is substituted with either an A, C, D, E, F, G, H, I, K, L, M, N, P, Q, R, S, T, W, or Y amino acid residue; wherein L345 is substituted with either an A, C, D, E, F, G, H, I, K, M, N, P, Q, R, S, T, V, W, or Y amino acid residue; wherein G346 is substituted with either an A, C, D, E, F, H, I, K, L, M, N, P, Q, R, S, T, V, W, or Y amino acid residue; wherein G347 is substituted with either an A, C, D, E, F, H, I, K, L, M, N, P, Q, R, S, T, V, W, or Y amino acid residue; wherein G348 is substituted with either an A, C, D, E, F, H, I, K, L, M, N, P, Q, R, S, T, V, W, or Y amino acid residue; wherein A349 is substituted with either a C, D, E, F, G, H, I, K, L, M, N, P, Q, R, S, T, V, W, or Y amino acid residue; wherein 1350 is substituted with either an A, C, D, E, F, G, H, K, L, M, N, P, Q, R, S, T, V, W, or Y amino acid residue; wherein C351 is substituted with either an A, D, E, F, G, H, I, K, L, M, N, P, Q, R, S, T, V, W, or Y amino acid residue; wherein M352 is substituted with either an A, C, D, E, F, G, H, I, K, L, N, P, Q, R, S, T, V, W, or Y amino acid residue; wherein E353 is substituted with either an A, C, D, F, G, H, I, K, L, M, N, P, Q, R, S, T, V, W, or Y amino acid residue; wherein L354 is substituted with either an A, C, D, E, F, G, H, 1, K, M, N, P, Q, R, S, T, V, W, or Y amino acid residue; wherein L355 is substituted with either an A, C, D, E, F, G, H, I, K, M, N, P, Q, R, S, T, V, W, or Y amino acid residue; wherein T356 is substituted with either an A, C, D, E, F, G, H, I, K, L, M, N, P, Q, R, S, V, W, or Y amino acid residue; wherein K357 is substituted with either an A, C, D, E, F, G, H, 1, L, M, N, P, Q, R, S, T, V, W, or Y amino acid residue; wherein Q358 is substituted with either an A, C, D, E, F, G, H, 1, K, L, M, N, P, R, S, T, V, W, or Y amino acid residue; wherein G359 is substituted with either an A, C, D, E, F, H, 1, K, L, M, N, P, Q, R, S, T, V, W, or Y amino acid residue; wherein W360 is substituted with either an A, C, D, E, F, G, H, 1, K, L, M, N, P, Q, R, S, T, V, or Y amino acid residue; wherein S361 is substituted with either an A, C, D, E, F, G, H, 1, K, L, M, N, P, Q, R, T, V, W, or Y amino acid residue; wherein S362 is substituted with either an A, C, D, E, F, G, H, 1, K, L, M, N, P, Q, R, T, V, W, or Y amino acid residue; wherein A363 is substituted with either a C, D, E, F, G, H, 1, K, L, M, N, P, Q, R, S, T, V, W, or Y amino acid residue; wherein Y364 is substituted with either an A, C, D, E, F, G, H, 1, K, L, M, N, P, Q, R, S, T, V, or W amino acid residue; wherein S365 is substituted with either an A, C, D, E, F, G, H, 1, K, L, M, N, P, Q, R, T, V, W, or Y amino acid residue; wherein 1366 is substituted with either an A, C, D, E, F, G, H, K, L, M, N, P, Q, R, S, T, V, W, or Y amino acid residue; wherein E367 is substituted with either an A, C, D, F, G, H, 1, K, L, M, N, P, Q, R, S, T, V, W, or Y amino acid residue; wherein S368 is substituted with either an A, C, D, E, F, G, H, 1, K, L, M, N, P, Q, R, T, V, W, or Y amino acid residue; wherein V369 is substituted with either an A, C, D, E, F, G, H, 1, K, L, M, N, P, Q, R, S, T, W, or Y amino acid residue; wherein 1370 is substituted with either an A, C, D, E, F, G, H, K, L, M, N, P, Q, R, S, T, V, W, or Y amino acid residue; wherein M371 is substituted with either an A, C, D, E, F, G, H, 1, K, L, N, P, Q, R, S, T, V, W, or Y amino acid residue; wherein Q372 is substituted with either an A, C, D, E, F, G, H, 1, K, L, M, N, P, R, S, T, V, W, or Y amino acid residue; wherein 1373 is substituted with either an A, C, D, E, F, G, H, K, L, M, N, P, Q, R, S, T, V, W, or Y amino acid residue; wherein S374 is substituted with either an A, C, D, E, F, G, H, I, K, L, M, N, P, Q, R, T, V, W, or Y amino acid residue; wherein A375 is substituted with either a C, D, E, F, G, H, I, K, L, M, N, P, Q, R, S, T, V, W, or Y amino acid residue; wherein T376 is substituted with either an A, C, D, E, F, G, H, I, K, L, M, N, P, Q, R, S, V, W, or Y amino acid residue; wherein L377 is substituted with either an A, C, D, E, F, G, H, I, K, M, N, P, Q, R, S, T, V, W, or Y amino acid residue; wherein V378 is substituted with either an A, C, D, E, F, G, H, I, K, L, M, N, P, Q, R, S, T, W, or Y amino acid residue; wherein K379 is substituted with either an A, C, D, E, F, G, H, I, L, M, N, P, Q, R, S, T, V, W, or Y amino acid residue; wherein G380 is substituted with either an A, C, D, E, F, H, I, K, L, M, N, P, Q, R, S, T, V, W, or Y amino acid residue; wherein K381 is substituted with either an A, C, D, E, F, G, H, I, L, M, N, P, Q, R, S, T, V, W, or Y amino acid residue; wherein A382 is substituted with either a C, D, E, F, G, H, I, K, L, M, N, P, Q, R, S, T, V, W, or Y amino acid residue; wherein R383 is substituted with either an A, C, D, E, F, G, H, I, K, L, M, N, P, Q, S, T, V, W, or Y amino acid residue; wherein V384 is substituted with either an A, C, D, E, F, G, H, I, K, L, M, N, P, Q, R, S, T, W, or Y amino acid residue; wherein Q385 is substituted with either an A, C, D, E, F, G, H, I, K, L, M, N, P, R, S, T, V, W, or Y amino acid residue; wherein F386 is substituted with either an A, C, D, E, G, H, I, K, L, M, N, P, Q, R, S, T, V, W, or Y amino acid residue; wherein G387 is substituted with either an A, C, D, E, F, H, I, K, L, M, N, P, Q, R, S, T, V, W, or Y amino acid residue; wherein A388 is substituted with either a C, D, E, F, G, H, I, K, L, M, N, P, Q, R, S, T, V, W, or Y amino acid residue; wherein N389 is substituted with either an A, C, D, E, F, G, H, I, K, L, M, P, Q, R, S, T, V, W, or Y amino acid residue; wherein K390 is substituted with either an A, C, D, E, F, G, H, I, L, M, N, P, Q, R, S, T, V, W, or Y amino acid residue; wherein S391 is substituted with either an A, C, D, E, F, G, H, I, K, L, M, N, P, Q, R, T, V, W, or Y amino acid residue; wherein Q392 is substituted with either an A, C, D, E, F, G, H, I, K, L, M, N, P, R, S, T, V, W, or Y amino acid residue; wherein Y393 is substituted with either an A, C, D, E, F, G, H, I, K, L, M, N, P, Q, R, S, T, V, or W amino acid residue; wherein S394 is substituted with either an A, C, D, E, F, G, H, I, K, L, M, N, P, Q, R, T, V, W, or Y amino acid residue; wherein L395 is substituted with either an A, C, D, E, F, G, H, I, K, M, N, P, Q, R, S, T, V, W, or Y amino acid residue; wherein T396 is substituted with either an A, C, D, E, F, G, H, I, K, L, M, N, P, Q, R, S, V, W, or Y amino acid residue; wherein R397 is substituted with either an A, C, D, E, F, G, H, I, K, L, M, N, P, Q, S, T, V, W, or Y amino acid residue; wherein A398 is substituted with either a C, D, E, F, G, H, I, K, L, M, N, P, Q, R, S, T, V, W, or Y; wherein Q399 is substituted with either an A, C, D, E, F, G, H, I, K, L, M, N, P, R, S, T, V, W, or Y amino acid residue; wherein Q400 is substituted with either an A, C, D, E, F, G, H, I, K, L, M, N, P, R, S, T, V, W, or Y amino acid residue; wherein S401 is substituted with either an A, C, D, E, F, G, H, I, K, L, M, N, P, Q, R, T, V, W, or Y amino acid residue; wherein Y402 is substituted with either an A, C, D, E, F, G, H, I, K, L, M, N, P, Q, R, S, T, V, or W amino acid residue; wherein K403 is substituted with either an A, C, D, E, F, G, H, I, L, M, N, P, Q, R, S, T, V, W, or Y amino acid residue; wherein S404 is substituted with either an A, C, D, E, F, G, H, I, K, L, M, N, P, Q, R, T, V, W, or Y amino acid residue; wherein L405 is substituted with either an A, C, D, E, F, G, H, I, K, M, N, P, Q, R, S, T, V, W, or Y amino acid residue; wherein V406 is substituted with either an A, C, D, E, F, G, H, I, K, L, M, N, P, Q, R, S, T, W, or Y amino acid residue; wherein Q407 is substituted with either an A, C, D, E, F, G, H, I, K, L, M, N, P, R, S, T, V, W, or Y amino acid residue; wherein 1408 is substituted with either an A, C, D, E, F, G, H, K, L, M, N, P, Q, R, S, T, V, W, or Y amino acid residue; wherein H409 is substituted with either an A, C, D, E, F, G, I, K, L, M, N, P, Q, R, S, T, V, W, or Y amino acid residue; wherein E410 is substituted with either an A, C, D, F, G, H, I, K, L, M, N, P, Q, R, S, T, V, W, or Y amino acid residue; and/or wherein K411 is substituted with either an A, C, D, E, F, G, H, I, L, M, N, P, Q, R, S, T, V, W, or Y amino acid residue of SEQ ID NO:2, in addition to any combination thereof. The present invention also encompasses the use of one or more of these RATL1d6 UBC domain amino acid substituted polypeptides as immunogenic and/or antigenic epitopes as described elsewhere herein.

In other preferred embodiments, the following RATL1d6 UBC domain conservative amino acid substitutions are encompassed by the present invention: wherein G248 is substituted with either an A, M, S, or T; wherein S249 is substituted with either an A, G, M, or T; wherein V250 is substituted with either an A, I, or L; wherein Q251 is substituted with a N; wherein A252 is substituted with either a G, I, L, M, S, T, or V; wherein T253 is substituted with either an A, G, M, or S; wherein D254 is substituted with an E; wherein R255 is substituted with either a K, or H; wherein L256 is substituted with either an A, 1, or V; wherein M257 is substituted with either an A, G, S, or T; wherein K258 is substituted with either a R, or H; wherein E259 is substituted with a D; wherein L260 is substituted with either an A, I, or V; wherein R261 is substituted with either a K, or H; wherein D262 is substituted with an E; wherein 1263 is substituted with either an A, V, or L; wherein Y264 is either an F, or W; wherein R265 is substituted with either a K, or H; wherein S266 is substituted with either an A, G, M, or T; wherein Q267 is substituted with a N; wherein S268 is substituted with either an A, G, M, or T; wherein F269 is substituted with either a W, or Y; wherein K270 is substituted with either a R, or H; wherein G271 is substituted with either an A, M, S, or T; wherein G272 is substituted with either an A, M, S, or T; wherein N273 is substituted with a Q; wherein Y274 is either an F, or W; wherein A275 is substituted with either a G, I, L, M, S, T, or V; wherein V276 is substituted with either an A, I, or L; wherein E277 is substituted with a D; wherein L278 is substituted with either an A, I, or V; wherein V279 is substituted with either an A, I, or L; wherein N280 is substituted with a Q; wherein D281 is substituted with an E; wherein S282 is substituted with either an A, G, M, or T; wherein L283 is substituted with either an A, I, or V; wherein Y284 is either an F, or W; wherein D285 is substituted with an E; wherein W286 is either an F, or Y; wherein N287 is substituted with a Q; wherein V288 is substituted with either an A, 1, or L; wherein K289 is substituted with either a R, or H; wherein L290 is substituted with either an A, 1, or V; wherein L291 is substituted with either an A, 1, or V; wherein K292 is substituted with either a R, or H; wherein V293 is substituted with either an A, 1, or L; wherein D294 is substituted with an E; wherein Q295 is substituted with a N; wherein D296 is substituted with an E; wherein S297 is substituted with either an A, G, M, or T; wherein A298 is substituted with either a G, I, L, M, S, T, or V; wherein L299 is substituted with either an A, 1, or V; wherein H300 is substituted with either a K, or R; wherein N301 is substituted with a Q; wherein D302 is substituted with an E; wherein L303 is substituted with either an A, I, or V; wherein Q304 is substituted with a N; wherein 1305 is substituted with either an A, V, or L; wherein L306 is substituted with either an A, I, or V; wherein K307 is substituted with either a R, or H; wherein E308 is substituted with a D; wherein K309 is substituted with either a R, or H; wherein E310 is substituted with a D; wherein G311 is substituted with either an A, M, S, or T; wherein A312 is substituted with either a G, I, L, M, S, T, or V; wherein D313 is substituted with an E; wherein F314 is substituted with either a W, or Y; wherein 1315 is substituted with either an A, V, or L; wherein L316 is substituted with either an A, I, or V; wherein L317 is substituted with either an A, I, or V; wherein N318 is substituted with a Q; wherein F319 is substituted with either a W, or Y; wherein S320 is substituted with either an A, G, M, or T; wherein F321 is substituted with either a W, or Y; wherein K322 is substituted with either a R, or H; wherein D323 is substituted with an E; wherein N324 is substituted with a Q; wherein F325 is substituted with either a W, or Y; wherein P326 is a P; wherein F327 is substituted with either a W, or Y; wherein D328 is substituted with an E; wherein P329 is a P; wherein P330 is a P; wherein F331 is substituted with either a W, or Y; wherein V332 is substituted with either an A, I, or L; wherein R333 is substituted with either a K, or H; wherein V334 is substituted with either an A, I, or L; wherein V335 is substituted with either an A, I, or L; wherein S336 is substituted with either an A, G, M, or T; wherein P337 is a P; wherein V338 is substituted with either an A, I, or L; wherein L339 is substituted with either an A, I, or V; wherein S340 is substituted with either an A, G, M, or T; wherein G341 is substituted with either an A, M, S, or T; wherein G342 is substituted with either an A, M, S, or T; wherein Y343 is either an F, or W; wherein V344 is substituted with either an A, I, or L; wherein L345 is substituted with either an A, I, or V; wherein G346 is substituted with either an A, M, S, or T; wherein G347 is substituted with either an A, M, S, or T; wherein G348 is substituted with either an A, M, S, or T; wherein A349 is substituted with either a G, I, L, M, S, T, or V; wherein 1350 is substituted with either an A, V, or L; wherein C351 is a C; wherein M352 is substituted with either an A, G, S, or T; wherein E353 is substituted with a D; wherein L354 is substituted with either an A, I, or V; wherein L355 is substituted with either an A, 1, or V; wherein T356 is substituted with either an A, G, M, or S; wherein K357 is substituted with either a R, or H; wherein Q358 is substituted with a N; wherein G359 is substituted with either an A, M, S, or T; wherein W360 is either an F, or Y; wherein S361 is substituted with either an A, G, M, or T; wherein S362 is substituted with either an A, G, M, or T; wherein A363 is substituted with either a G, I, L, M, S, T, or V; wherein Y364 is either an F, or W; wherein S365 is substituted with either an A, G, M, or T; wherein 1366 is substituted with either an A, V, or L; wherein E367 is substituted with a D; wherein S368 is substituted with either an A, G, M, or T; wherein V369 is substituted with either an A, I, or L; wherein 1370 is substituted with either an A, V, or L; wherein M371 is substituted with either an A, G, S, or T; wherein Q372 is substituted with a N; wherein 1373 is substituted with either an A, V, or L; wherein S374 is substituted with either an A, G, M, or T; wherein A375 is substituted with either a G, I, L, M, S, T, or V; wherein T376 is substituted with either an A, G, M, or S; wherein L377 is substituted with either an A, I, or V; wherein V378 is substituted with either an A, I, or L; wherein K379 is substituted with either a R, or H; wherein G380 is substituted with either an A, M, S, or T; wherein K381 is substituted with either a R, or H; wherein A382 is substituted with either a G, I, L, M, S, T, or V; wherein R383 is substituted with either a K, or H; wherein V384 is substituted with either an A, I, or L; wherein Q385 is substituted with a N; wherein F386 is substituted with either a W, or Y; wherein G387 is substituted with either an A, M, S, or T; wherein A388 is substituted with either a G, I, L, M, S, T, or V; wherein N389 is substituted with a Q; wherein K390 is substituted with either a R, or H; wherein S391 is substituted with either an A, G, M, or T; wherein Q392 is substituted with a N; wherein Y393 is either an F, or W; wherein S394 is substituted with either an A, G, M, or T; wherein L395 is substituted with either an A, I, or V; wherein T396 is substituted with either an A, G, M, or S; wherein R397 is substituted with either a K, or H; wherein A398 is substituted with either a G, I, L, M, S, T, or V; wherein Q399 is substituted with a N; wherein Q400 is substituted with a N; wherein S401 is substituted with either an A, G, M, or T; wherein Y402 is either an F, or W; wherein K403 is substituted with either a R, or H; wherein S404 is substituted with either an A, G, M, or T; wherein L405 is substituted with either an A, I, or V; wherein V406 is substituted with either an A, I, or L; wherein Q407 is substituted with a N; wherein 1408 is substituted with either an A, V, or L; wherein H409 is substituted with either a K, or R; wherein E410 is substituted with a D; and/or wherein K411 is substituted with either a R, or H of SEQ ID NO:2 in addition to any combination thereof. Other suitable substitutions within the RATL1d6 UBC domain are encompassed by the present invention and are referenced elsewhere herein. The present invention also encompasses the use of one or more of these RATL1 d6 UBC domain conservative amino acid substituted polypeptides as immunogenic and/or antigenic epitopes as described elsewhere herein.

To express a biologically active RATL1d6 polypeptide or peptide, the nucleotide sequences encoding RATL1d6 polypeptide, or functional equivalents, may be inserted into an appropriate expression vector, i.e., a vector which contains the necessary elements for the transcription and translation of the inserted coding sequence.

Methods which are well known to those skilled in the art may be used to construct expression vectors containing sequences encoding RATL1d6 polypeptide and appropriate transcriptional and translational control elements. These methods include in vitro recombinant DNA techniques, synthetic techniques, and in vivo genetic recombination. Such techniques are described in J. Sambrook et al., 1989, Molecular Cloning, A Laboratory Manual, Cold Spring Harbor Press, Plainview, N.Y. and in F. M. Ausubel et al., 1989, Current Protocols in Molecular Biology, John Wiley & Sons, New York, N.Y.

A variety of expression vector/host systems may be utilized to contain and express sequences encoding RATL1d6 polypeptide. Such expression vector/host systems include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus (CaMV) and tobacco mosaic virus (TMV)), or with bacterial expression vectors (e.g., Ti or pBR322 plasmids); or animal cell systems. The host cell employed is not limiting to the present invention.

“Control elements” or “regulatory sequences” are those non-translated regions of the vector, e.g., enhancers, promoters, 5′ and 3′ untranslated regions, which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the BLUESCRIPT phagemid (Stratagene, La Jolla, Calif.) or PSPORT1 plasmid (Life Technologies), and the like, may be used. The baculovirus polyhedrin promoter may be used in insect cells. Promoters or enhancers derived from the genomes of plant cells (e.g., heat shock, RUBISCO; and storage protein genes), or from plant viruses (e.g., viral promoters or leader sequences), may be cloned into the vector. In mammalian cell systems, promoters from mammalian genes or from mammalian viruses are preferred. If it is necessary to generate a cell line that contains multiple copies of the sequence encoding RATL1d6, vectors based on SV40 or EBV may be used with an appropriate selectable marker.

In bacterial systems, a number of expression vectors may be selected, depending upon the use intended for the expressed RATL1d6 product. For example, when large quantities of expressed protein are needed for the induction of antibodies, vectors which direct high level expression of fusion proteins that are readily purified may be used. Such vectors include, but are not limited to, the multifunctional E. coli cloning and expression vectors such as BLUESCRIPT (Stratagene), in which the sequence encoding RATL1d6 polypeptide may be ligated into the vector in-frame with sequences for the amino-terminal Met and the subsequent 7 residues of β-galactosidase, so that a hybrid protein is produced; pIN vectors (See, G. Van Heeke and S.M. Schuster, 1989, J. Biol. Chem., 264:5503-5509); and the like. pGEX vectors (Promega, Madison, Wis.) may also be used to express foreign polypeptides, as fusion proteins with glutathione S-transferase (GST). In general, such fusion proteins are soluble and can be easily purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast, Saccharomyces cerevisiae, a number of vectors containing constitutive or inducible promoters such as alpha factor, alcohol oxidase, and PGH may be used. (For reviews, see F. M. Ausubel et al., supra, and Grant et al., 1987, Methods Enzymol., 153:516-544).

Should plant expression vectors be desired and used, the expression of sequences encoding RATL1d6 polypeptide may be driven by any of a number of promoters. For example, viral promoters such as the ³⁵S and 19S promoters of CaMV may be used alone or in combination with the omega leader sequence from TMV (N. Takamatsu, 1987, EMBO J., 6:307-311). Alternatively, plant promoters such as the small subunit of RUBISCO, or heat shock promoters, may be used (G. Coruzzi et al., 1984, EMBO J., 3:1671-1680; R. Broglie et al., 1984, Science, 224:838-843; and J. Winter et al., 1991, Results Probl. Cell Differ. 17:85-105). These constructs can be introduced into plant cells by direct DNA transformation or pathogen-mediated transfection. Such techniques are described in a number of generally available reviews (See, for example, S. Hobbs or L. E. Murry, In: McGraw Hill Yearbook of Science and Technology (1992) McGraw Hill, New York, N.Y.; pp. 191-196).

An insect system may also be used to express RATL1d6 polypeptide For example, in one such system, Autographa californica nuclear polyhedrosis virus (AcNPV) is used as a vector to express foreign genes in Spodoptera frugiperda cells or in Trichoplusia larvae. The sequences encoding RATL1d6 polypeptide may be cloned into a non-essential region of the virus such as the polyhedrin gene and placed under control of the polyhedrin promoter. Successful insertion of RATL1d6 polypeptide will render the polyhedrin gene inactive and produce recombinant virus lacking coat protein. The recombinant viruses may then be used to infect, for example, S. frugiperda cells or Trichoplusia larvae in which the RATL1d6 polypeptide product may be expressed (E. K. Engelhard et al., 1994, Proc. Nat. Acad. Sci., 91:3224-3227).

In mammalian host cells, a number of viral-based expression systems may be utilized. In cases where an adenovirus is used as an expression vector, sequences encoding RATL1d6 polypeptide may be ligated into an adenovirus transcription/translation complex containing the late promoter and tripartite leader sequence. Insertion in a non-essential E1 or E3 region of the viral genome may be used to obtain a viable virus which is capable of expressing RATL1d6 polypeptide in infected host cells (J. Logan and T. Shenk, 1984, Proc. Natl. Acad. Sci., 81:3655-3659). In addition, transcription enhancers, such as the Rous sarcoma virus (RSV) enhancer, may be used to increase expression in mammalian host cells.

Specific initiation signals may also be used to achieve more efficient translation of sequences encoding RATL1d6 polypeptide. Such signals include the ATG initiation codon and adjacent sequences. In cases where sequences encoding RATL1d6 polypeptide, its initiation codon, and upstream sequences are inserted into the appropriate expression vector, no additional transcriptional or translational control signals may be needed. However, in cases where only coding sequence, or a fragment thereof, is inserted, exogenous translational control signals, including the ATG initiation codon, should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system that is used, such as those described in the literature (D. Scharf et al., 1994, Results Probl. Cell Differ., 20:125-162).

Moreover, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding and/or function. Different host cells having specific cellular machinery and characteristic mechanisms for such post-translational activities (e.g., CHO, HeLa, MDCK, HEK293, and W138) are available from the American Type Culture Collection (ATCC), American Type Culture Collection (ATCC), 10801 University Boulevard, Manassas, Va. 20110-2209, and may be chosen to ensure the correct modification and processing of the foreign protein.

For long-term, high-yield production of recombinant proteins, stable expression is preferred. For example, cell lines which stably express RATL1d6 protein may be transformed using expression vectors which may contain viral origins of replication and/or endogenous expression elements and a selectable marker gene on the same, or on a separate, vector. Following the introduction of the vector, cells may be allowed to grow for 1-2 days in an enriched cell culture medium before they are switched to selective medium. The purpose of the selectable marker is to confer resistance to selection, and its presence allows the growth and recovery of cells which successfully express the introduced sequences. Resistant clones of stably transformed cells may be proliferated using tissue culture techniques appropriate to the cell type.

Any number of selection systems may be used to recover transformed cell lines. These include, but are not limited to, the Herpes Simplex Virus thymidine kinase (HSV TK), (M. Wigler et al., 1977, Cell, 11:223-32) and adenine phosphoribosyltransferase (I. Lowy et al., 1980, Cell, 22:817-23) genes which can be employed in tk⁻ or aprt⁻ cells, respectively. Also, anti-metabolite, antibiotic or herbicide resistance can be used as the basis for selection; for example, dhfr, which confers resistance to methotrexate (M. Wigler et al., 1980, Proc. Natl. Acad. Sci., 77:3567-70); npt, which confers resistance to the aminoglycosides neomycin and G-418 (F. Colbere-Garapin et al., 1981, J. Mol. Biol., 150:1-14); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (S. C. Hartman and R. C. Mulligan, 1988, Proc. Nat. Acad. Sci., 85:8047-51). Recently, the use of visible markers has gained popularity with such markers as the anthocyanins, β-glucuronidase and its substrate GUS, and luciferase and its substrate luciferin, which are widely used not only to identify transformants, but also to quantify the amount of transient or stable protein expression that is attributable to a specific vector system (C.A. Rhodes et al., 1995, Methods Mol. Biol., 55:121-131).

Although the presence/absence of marker gene expression suggests that the gene of interest is also present, the presence and expression of the desired gene of interest may need to be confirmed. For example, if the nucleic acid sequence encoding RATL1d6 polypeptide is inserted within a marker gene sequence, recombinant cells containing sequences encoding RATL1d6 polypeptide can be identified by the absence of marker gene function. Alternatively, a marker gene can be placed in tandem with a sequence encoding RATL1d76 polypeptide under the control of a single promoter. Expression of the marker gene in response to induction or selection usually indicates co-expression of the tandem gene.

Alternatively, host cells which contain the nucleic acid sequence encoding RATL1d6 polypeptide and which express RATL1d6 polypeptide product may be identified by a variety of procedures known to those having skill in the art. These procedures include, but are not limited to, DNA-DNA or DNA-RNA hybridizations and protein bioassay or immunoassay techniques, including membrane, solution, or chip based technologies, for the detection and/or quantification of nucleic acid or protein.

The presence of polynucleotide sequences encoding RATL1d6 polypeptide can be detected by DNA-DNA or DNA-RNA hybridization, or by amplification using probes or portions or fragments of polynucleotides encoding RATL1d6 polypeptide. Nucleic acid amplification based assays involve the use of oligonucleotides or oligomers, based on the sequences encoding RATL1d6 polypeptide, to detect transformants containing DNA or RNA encoding RATL1d6 polypeptide.

A wide variety of labels and conjugation techniques are known and employed by those skilled in the art and may be used in various nucleic acid and amino acid assays. Means for producing labeled hybridization or PCR probes for detecting sequences related to polynucleotides encoding RATL1d6 polypeptide include oligo-labeling, nick translation, end-labeling, or PCR amplification using a labeled nucleotide. Alternatively, the sequences encoding RATL1d6 polypeptide, or any portions or fragments thereof, may be cloned into a vector for the production of an mRNA probe. Such vectors are known in the art, are commercially available, and may be used to synthesize RNA probes in vitro by addition of an appropriate RNA polymerase, such as T7, T3, or SP(6) and labeled nucleotides. These procedures may be conducted using a variety of commercially available kits (e.g., Amersham Pharmacia Biotech, Promega and U.S. Biochemical Corp.). Suitable reporter molecules or labels which may be used include radionuclides, enzymes, fluorescent, chemiluminescent, or chromogenic agents, as well as substrates, cofactors, inhibitors, magnetic particles, and the like.

Host cells transformed with nucleotide sequences encoding RATL1d6 protein, or fragments thereof, may be cultured under conditions suitable for the expression and recovery of the protein from cell culture. The protein produced by a recombinant cell may be secreted or contained intracellularly depending on the sequence and/or the vector used. As will be understood by those having skill in the art, expression vectors containing polynucleotides which encode RATL1d6 protein may be designed to contain signal sequences which direct secretion of the RATL1d6 protein through a prokaryotic or eukaryotic cell membrane. Other constructions may be used to join nucleic acid sequences encoding RATL1d6 protein to nucleotide sequence encoding a polypeptide domain which will facilitate purification of soluble proteins. Such purification facilitating domains include, but are not limited to, metal chelating peptides such as histidine-tryptophan modules that allow purification on immobilized metals; protein A domains that allow purification on immobilized immunoglobulin; and the domain utilized in the FLAGS extension/affinity purification system (Immunex Corp., Seattle, Wash.). The inclusion of cleavable linker sequences such as those specific for Factor XA or enterokinase (Invitrogen, San Diego, Calif.) between the purification domain and RATL1d6 protein may be used to facilitate purification. One such expression vector provides for expression of a fusion protein containing RATL1d6 and a nucleic acid encoding 6 histidine residues preceding a thioredoxin or an enterokinase cleavage site. The histidine residues facilitate purification on IMAC (immobilized metal ion affinity chromatography) as described by J. Porath et al., 1992, Prot. Exp. Purif, 3:263-281, while the enterokinase cleavage site provides a means for purifying from the fusion protein. For a discussion of suitable vectors for fusion protein production, see D. J. Kroll et al., 1993; DNA Cell Biol., 12:441-453.

In addition to recombinant production, fragments of RATL1d6 polypeptide may be produced by direct peptide synthesis using solid-phase techniques (J. Merrifield, 1963, J. Am. Chem. Soc., 85:2149-2154). Protein synthesis may be performed using manual techniques or by automation. Automated synthesis may be achieved, for example, using ABI 431A Peptide Synthesizer (PE Biosystems). Various fragments of RATL1d6 polypeptide can be chemically synthesized separately and then combined using chemical methods to produce the full length molecule.

Human artificial chromosomes (HACs) may be used to deliver larger fragments of DNA than can be contained and expressed in a plasmid vector. HACs are linear microchromosomes which may contain DNA sequences of 10K to 10M in size, and contain all of the elements that are required for stable mitotic chromosome segregation and maintenance (See, J. J. Harrington et al., 1997, Nature Genet., 15:345-355). HACs of 6 to 10M are constructed and delivered via conventional delivery methods (e.g., liposomes, polycationic amino polymers, or vesicles) for therapeutic purposes.

A variety of protocols for detecting and measuring the expression of RATL1d6 polypeptide using either polyclonal or monoclonal antibodies specific for the protein are known and practiced in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive with two non-interfering epitopes on the RATL1d6 polypeptide is preferred, but a competitive binding assay may also be employed. These and other assays are described in the art as represented by the publication of R. Hampton et al., 1990; Serological Methods, a Laboratory Manual, APS Press, St Paul, Minn. and D. E. Maddox et al., 1983; J. Exp. Med., 158:1211-1216). Transmembrane domain regions

The RATL1d6 polypeptide was determined to comprise two transmembrane domains, one located from about amino acid 69 to about amino acid 88, and the other located from about amino acid 334 to about amino acid 356 of SEQ ID NO:2. In this context, the term “about” can be construed to mean 1, 2, 3, 4, 5, 6, 7, 8, 9, or 10 amino acids beyond the N-terminus and/or C-terminus of the above referenced transmembrane domain polypeptides. The TMPRED program was used for transmembrane prediction (K Hofmann and W Stoffel, 1993, Biol. Chem., 347:166).

In a preferred embodiment, the following transmembrane domain polypeptide is encompassed by the present invention: PHLPPRGSVPGDPVRIHCNITESYPAVPPIWSVESDDPNLAAVLERLVDIKK GNTLLLQHLKRIISDLCKLYNLPQHPDVEMLDQPLPAEQCTQEDVSSEDED EEMPEDTEDLDHYEMKEEEPAEGKKSEDDGIGKENLAILEKIKKNQRQDYL NGAVSGSVQATDRLMKELRDIYRSQSFKGGNYAVELVNDSLYDWNVKLLK VDQDSALHNDLQILKEKEGADFILLNFSFKDNFPFDPPFVR (SEQ ID NO:17). Polynucleotides encoding this polypeptide is also provided. The present invention also encompasses the use of the RATL1d6 transmembrane domain polypeptide as an immunogenic and/or antigenic epitope, or the source of such epitopes, as described elsewhere herein.

In preferred embodiments, the following N-terminal RATL1d6 inter-transmembrane domain deletion polypeptides are encompassed by the present invention: P1-R245, H2-R245, L3-R245, P4-R245, P5-R245, R6-R245, G7-R245, S8-R245, V9-R245, P10-R245, G11-R245, D12-R245, P13-R245, V14-R245, R15-R245, I16-R245, H17-R245, C18-R245, N19-R245, I20-R245, T21-R245, E22-R245, S23-R245, Y24-R245, P25-R245, A26-R245, V27-R245, P28-R245, P29-R245, I30-R245, W31-R245, S32-R245, V33-R245, E34-R245, S35-R245, D36-R245, D37-R245, P38-R245, N39-R245, L40-R245, A41-R245, A42-R245, V43-R245, L44-R245, E45-R245, R46-R245, L47-R245, V48-R245, D49-R245, I50-R245, K51-R245, K52-R245, G53-R245, N54-R245, T55-R245, L56-R245, L57-R245, L58-R245, Q59-R245, H60-R245, L61-R245, K62-R245, R63-R245, I64-R245, 165-R245, S66-R245, D67-R245, L68-R245, C69-R245, K70-R245, L71-R245, Y72-R245, N73-R245, L74-R245, P75-R245, Q76-R245, H77-R245, P78-R245, D79-R245, V80-R245, E81-R245, M82-R245, L83-R245, D84-R245, Q85-R245, P86-R245, L87-R245, P88-R245, A89-R245, E90-R245, Q91-R245, C92-R245, T93-R245, Q94-R245, E95-R245, D96-R245, V97-R245, S98-R245, S99-R245, E100-R245, D101-R245, E102-R245, D103-R245, E104-R245, E105-R245, M106-R245, P107-R245, E108-R245, D109-R245, T110-R245, E111-R245, D112-R245, L113-R245, D114-R245, H115-R245, Y116-R245, E117-R245, M118-R245, K119-R245, E120-R245, E121-R245, E122-R245, P123-R245, A124-R245, E125-R245, G126-R245, K127-R245, K128-R245, S129-R245, E130-R245, D131-R245, D132-R245, G133-R245, I134-R245, G135-R245, K136-R245, E137-R245, N138-R245, L139-R245, A140-R245, I141-R245, L142-R245, E143-R245, K144-R245, 1145-R245, K146-R245, K147-R245, N148-R245, Q149-R245, R150-R245, Q151-R245, D152-R245, Y153-R245, L154-R245, N155-R245, G156-R245, A157-R245, V158-R245, S159-R245, G160-R245, S161-R245, V162-R245, Q163-R245, A164-R245, T165-R245, D166-R245, R167-R245, L168-R245, M169-R245, K170-R245, E171-R245, L172-R245, R173-R245, D174-R245, 1175-R245, Y176-R245, R177-R245, S178-R245, Q179-R245, S180-R245, F181-R245, K182-R245, G183-R245, G184-R245, N185-R245, Y186-R245, A187-R245, V188-R245, E189-R245, L190-R245, V191-R245, N192-R245, D193-R245, S194-R245, L195-R245, Y196-R245, D197-R245, W198-R245, N199-R245, V200-R245, K201-R245, L202-R245, L203-R245, K204-R245, V205-R245, D206-R245, Q207-R245, D208-R245, S209-R245, A210-R245, L211-R245, H212-R245, N213-R245, D214-R245, L215-R245, Q216-R245, I217-R245, L218-R245, K219-R245, E220-R245, K221-R245, E222-R245, G223-R245, A224-R245, D225-R245, F226-R245, I227-R245, L228-R245, L229-R245, N230-R245, F231-R245, S232-R245, F233-R245, K234-R245, D235-R245, N236-R245, F237-R245, P238-R245, and/or F239-R245 of SEQ ID NO:2. Polynucleotide sequences encoding these polypeptides are also provided. The present invention also encompasses the use of these N-terminal RATL1d6 inter-transmembrane domain deletion polypeptides as immunogenic and/or antigenic epitopes as described elsewhere herein.

In other preferred embodiments, the following C-terminal RATL1d6 inter-transmembrane domain deletion polypeptides are encompassed by the present invention: P1-R245, P1-V244, P1-F243, P1-P242, P1-P241, P1-D240, P1-F239, P1-P238, P1-F237, P1-N236, P1-D235, P1-K234, P1-F233, P1-S232, P1-F231, P1-N230, P1-L229, P1-L228, P1-1227, P1-F226, P1-D225, P1-A224, P1-G223, P1-E222, P1-K221, P1-E220, P1-K219, P1-L218, P1-I217, P1-Q216, P1-L215, P1-D214, P1-N213, P1-H212, P1-L211, P1-A210, P1-S209, P1-D208, P1-Q207, P1-D206, P1-V205, P1-K204, P1-L203, P1-L202, P1-K201, P1-V200, P1-N199, P1-W198, P1-D197, P1-Y196, P1-L195, P1-S194, P1-D193, P1-N192, P1-V191, P1-L190, P1-E189, P1-V188, P1-A187, P1-Y186, P1-N185, P1-G184, P1-G183, P1-K182, P1-F181, P1-S180, P1-Q179, P1-S178, P1-R177, P1-Y176, P1-I175, P1-D174, P1-R173, P1-L172, P1-E171, P1-K170, P1-M169, P1-L168, P1-R167, P1-D166, P1-T165, P1-A164, P1-Q163, P1-V162, P1-S161, P1-G160, P1-S159, P1-V158, P1-A157, P1-G156, P1-N155, P1-L154, P1-Y153, P1-D152, P1-Q151, P1-R150, P1-Q149, P1-N148, P1-K147, P1-K146, P1-I145, P1-K144, P1-E143, P1-L142, P1-I141, P1-A140, P1-L139, P1-N138, P1-E137, P1-K136, P1-G135, P1-I134, P1-G133, P1-D132, P1-D131, P1-E130, P1-S129, P1-K128, P1-K127, P1-G126, P1-E125, P1-A124, P1-P123, P1-E122, P1-E121, P1-E120, P1-K119, P1-M118, P1-E117, P1-Y116, P1-H115, P1-D114, P1-L113, P1-D112, P1-E111, P1-T110, P1-D109, P1-E108, P1-P107, P1-M106, P1-E105, P1-E104, P1-D103, P1-E102, P1-D101, P1-E100, P1-S99, P1-S98, P1-V97, P1-D96, P1-E95, P1-Q94, P1-T93, P1-C92, P1-Q91, P1-E90, P1-A89, P1-P88, P1-L87, P1-P86, P1-Q85, P1-D84, P1-L83, P1-M82, P1-E81, P1-V80, P1-D79, P1-P78, P1-H77, P1-Q76, P1-P75, P1-L74, P1-N73, P1-Y72, P1-L71, P1-K70, P1-C69, P1-L68, P1-D67, P1-S66, P1-165, P1-164, P1-R63, P1-K62, P1-L61, P1-H60, P1-Q59, P1-L58, P1-L57, P1-L56, P1-T55, P1-N54, P1-G53, P1-K52, P1-K51, P1-150, P1-D49, P1-V48, P1-L47, P1-R46, P1-E45, P1-L44, P1-V43, P1-A42, P1-A41, P1-L40, P1-N39, P1-P38, P1-D37, P1-D36, P1-S35, P1-E34, P1-V33, P1-S32, P1-W31, P1-130, P1-P29, P1-P28, P1-V27, P1-A26, P1-P25, P1-Y24, P1-S23, P1-E22, P1-T21, P1-120, P1-N19, P1-C18, P1-H₁₇, P1-116, P1-R15, P1-V14, P1-P13, P1-D12, P1-G11, P1-P10, P1-V9, P1-S8, and/or P1-G7 of SEQ ID NO:2. Polynucleotide sequences encoding these polypeptides are also provided. The present invention also encompasses the use of these C-terminal RATL1 d6 inter-transmembrane domain deletion polypeptides as immunogenic and/or antigenic epitopes as described elsewhere herein.

Therapeutics

RATL1d6 polypeptide shares homology with known ubiquitin conjugating enzymes and is thus provided as a new member of the UBC protein family. Because RATL1d6 was expressed in and isolated from activated T lymphocytes, the RATL1 d6 product may play a role in immune disorders, e.g., lymphoproliferative disease, for example, in cell cycle regulation, and/or in cell signaling. In a manner similar to that of other ubiquitin conjugating enzyme family members, the RATL1d6 protein may be further involved in neoplastic, developmental and neuronal disorders, where it may also be associated with cell cycle and cell signaling activities, as described further below. With specific regard to lymphoproliferative diseases and inflammation, inhibitors of the RATL1d6 protein may play a role as immunosuppressive agents, for example, by preventing entry of lymphocytic cells into the cell cycle, or by blocking intracellular signaling events. In addition, RATL1d6 inhibitors may serve as anti-inflammatory drugs.

Degradation of tumor suppressor proteins, such as p53, by E2 enzymes may contribute to the development of neoplastic disorders. Thus, in one embodiment of the present invention, an antagonist or inhibitor of RATL1d6 polypeptide may be administered to an individual to prevent or treat a neoplastic disorder. Such disorders may include, but are not limited to, adenocarcinoma, leukemia, lymphoma, melanoma, myeloma, sarcoma, and teratocarcinoma, and particularly, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus. In cancers or tumors of the above-origins, blocking ubiquitination might prolong the half-life, and therefore the function, of p53. In a related aspect, an antibody which specifically binds to RATL1d6 may be used directly as an antagonist or indirectly as a targeting or delivery mechanism for bringing a pharmaceutical agent to cells or tissue which express RATL1d6 polypeptide.

In a related embodiment, an inhibitor of RATL1d6 function may be useful as an anti-cancer drug or agent with particular regard to the treatment of lymphoproliferative diseases, or as an immunosuppressive drug by functioning as a dominant negative to a UBC such as the RATL1d6 protein product, in a manner similar to the tumor susceptibility gene TSG101, which has been found to be mutated at a high frequency in human breast cancers. (C. P. Ponting et al., 1997, J. Mol. Med., 75:467-469; L. Li et al., 1997, Cell, 88:143-154). TSG101 has been implicated as a tumor suppressor gene, encoding a product having homology to ubiquitin conjugating enzymes, but lacking the conserved cysteine that is typically present in E2 proteins and is necessary for enzyme function. (C.P. Ponting et al., supra). TSG101 has also been reported to function as a dominant negative regulator of the ubiquitination of short-lived proteins (C. P. Ponting et al, supra and E. V. Koonin and R. A. Abagyan, 1997, Nature Genetics, 16:330-331). Accordingly, an antagonist of certain UBCs, such as RATL1d6 of the present invention, may also act in a manner similar to that of the TSG101 product and be utilized in the treatment of cancers, including T-cell and B-cell lymphoproliferative disorders and/or as an agent to suppress adverse immune system reactions.

That RATL1d6 plays a negative role in the NF-KB pathway, a pathway of key importance in innate immunity, suggests that antagonists of this gene product could activate innate immunity. Innate immunity is the first line of defense against microbial pathogens including bacteria, fungi, viruses, etc. The cells of the immune system which are responsible for innate immunity are primarily macrophages/monocytes, and to a limited extent, neutrophils. Without wishing to be bound by theory, it is believed that antagonists of RATL1d6 could enhance the innate immune response and provide protection from invading pathogens in humans. In contrast, agonists of RATL1d6 would be expected to inhibit the NF-κB pathway and attenuate an inflammatory response. Hence, agonists of RATL1d6 may be useful in the treatment of inflammatory diseases including rheumatoid arthritis, asthma, multiple sclerosis, osteoarthritis, among others.

Since RATL1d6 was identified in a T-cell library, this suggests that the gene product may play a role in modulating an adaptive immune response, as well. Adaptive immune responses are primarily mediated by T-cells and require the processing and display of foreign, and in the case of autoimmune disease, native antigens. T-cell mediated responses are important in developing immunity after vaccination and also in eliminating tumor cells. Thus, it would be predicted that antagonists of RATL1d6 may enhance a person's immunity after vaccination. The RATL1d6 gene or gene product may also stimulate immune an immune response to tumors. In contrast, agonists of this gene could be useful for treating T-cell mediated autoimmune diseases such as rheumatoid arthritis, multiple sclerosis, psoriasis, among others.

The RATL1d6 protein, or functional portion thereof, can be employed in a method of suppressing the immune response in a patient, preferably human, who requires immunosuppression. For example, an antagonist or agonist can be administered to the patient in an amount effective to modulate the activity of the RATL1 d6 protein, or portion thereof, thereby causing an immunosuppressive effect. In the case of agonists or activators of RATL1d6 activity, immunosuppression may be caused by ubiquination of a cell receptor, preferably a T cell receptor, or component, or interactive component thereof, and subsequent down regulation of the receptor activity.

Abnormalities in processing of neural proteins (AP) by enzymes of the UCS may contribute to the cause of neuronal disorders. Since UCS are found in neuronal tissues, the RATL1d6 polypeptide, which appears to be a member of the family of proteins involved in UCS dependent proteolysis, may be affected, for example, by an antagonist of the RATL1d6 polypeptide. Accordingly, a RATL1d6 polypeptide antagonist may be administered to a subject to prevent or treat a neuronal disorder. Such disorders may include, but are not limited to, akathesia, Alzheimer's disease, amnesia, amyotrophic lateral sclerosis, bipolar disorder, catatonia, cerebral neoplasms, dementia, depression, Down's syndrome, tardive dyskinesia, dystonias, epilepsy, Huntington's disease, multiple sclerosis, Parkinson's disease, paranoid psychoses, schizophrenia, and Tourette's disorder.

In a preferred embodiment of the present invention, an antagonist or inhibitory agent of the RATL1d6 polypeptide may be administered to an individual to prevent or treat an immune disorder, or an immune-related disorder. Such disorders may include, but are not limited to, AIDS, Addison's disease, adult respiratory distress syndrome, allergies, anemia, asthma, atherosclerosis, bronchitis, cholecystitis, Crohn's disease, ulcerative colitis, atopic dermatitis, dermatomyositis, diabetes mellitus, emphysema, erythema nodosum, atrophic gastritis, glomerulonephritis, gout, Graves' disease, hypereosinophilia, irritable bowel syndrome, lupus erythematosus, multiple sclerosis, myasthenia gravis, myocardial or pericardial inflammation, osteoarthritis, osteoporosis, pancreatitis, polymyositis, rheumatoid arthritis, scleroderma, Sjogren's syndrome, and autoimmune thyroiditis; complications of cancer, hemodialysis, extracorporeal circulation; viral, bacterial, fungal, parasitic, protozoal, and helminthic infections and trauma. RATL1d6 inhibitors or antagonists may be utilized to prevent graft rejection, such as in solid organ or bone marrow transplants; or to prevent graft-versus-host disease following bone marrow transplantation.

In another embodiment of the present invention, an antagonist of RATL1d6 polypeptide may be administered to an individual in need thereof to prevent or treat a developmental disorder. Such disorders include, but are not limited to, renal tubular acidosis, Cushing's syndrome, achondroplastic dwarfism, Duchenne and Becker muscular dystrophy, gonadal dysgenesis, myelodysplastic syndrome, hereditary mucoepithelial dysplasia, hereditary keratodermas, hereditary neuropathies, such as Charcot-Marie-Tooth disease and neurofibromatosis, hypothyroidism, hydrocephalus, seizure disorders such as Syndenham's chorea and cerebral palsy, spina bifida, and congenital glaucoma, cataract, or sensorineural hearing loss.

In another embodiment of the present invention, an expression vector containing the complement of the polynucleotide encoding RATL1 d6 polypeptide may be administered to an individual to treat or prevent a neoplastic disorder, including, but not limited to, the types of cancers and tumors described above.

In another embodiment of the present invention, an expression vector containing the complement of the polynucleotide encoding RATL1d6 polypeptide may be administered to an individual to treat or prevent a neuronal disorder, including, but not limited to, the types of disorders described above.

In yet another embodiment of the present invention, an expression vector containing the complement of the polynucleotide encoding RATL1d6 polypeptide may be administered to an individual to treat or prevent an immune disorder, including, but not limited to, the types of immune disorders described above.

In a further embodiment of the present invention, an expression vector harboring the complement of the polynucleotide encoding RATL1d6 polypeptide may be administered to an individual to treat or prevent a developmental disorder, including, but not limited to, the types of disorders described above.

In another embodiment, the proteins, antagonists, antibodies, agonists, complementary sequences, or vectors of the present invention can be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

Antagonists or inhibitors of the RATL1d6 polypeptide of the present invention may be produced using methods which are generally known in the art. In particular, purified RATL1d6 protein, or fragments thereof, can be used to produce antibodies, or to screen libraries of pharmaceutical agents, to identify those which specifically bind RATL1d6, such as via high throughput screening techniques known and practiced in the art.

Antibodies specific for RATL1d6 polypeptide, or immunogenic peptide fragments thereof, can be generated using methods that have long been known and conventionally practiced in the art. Such antibodies may include, but are not limited to, polyclonal, monoclonal, chimeric, single chain, Fab fragments, and fragments produced by an Fab expression library. Neutralizing antibodies, (i.e., those which inhibit dimer formation) are especially preferred for therapeutic use. For polyclonal and/or monoclonal anti-RATL1d6 antibody production, the full-length RATL1d6 polypeptide can be utilized as an immunogen; alternatively, portions of the full-length polypeptide can be employed. Preferably, portions of the RATL1d6 polypeptide employed as immunogens include a portion that contains a domain, for example, the UBC (e.g., residues 246-422) or non-UBC (e.g., residues 1-245) domains of the protein.

For the production of antibodies, various hosts including goats, rabbits, sheep, rats, mice, humans, and others, can be immunized by injection with RATL1d6 polypeptide, or any fragment or oligopeptide thereof, which has immunogenic properties. Depending on the host species, various adjuvants may be used to increase the immunological response. Nonlimiting examples of suitable adjuvants include Freund's (complete or incomplete), RIBI, mineral gels such as aluminum hydroxide or silica, and surface active substances such as lysolecithin, pluronic polyols, polyanions, peptides, oil emulsions, KLH, and dinitrophenol. Adjuvants typically used in humans include BCG (bacilli Calmette Guerin) and Corynebacterium parvum.

Preferably, the peptides, fragments, or oligopeptides used to induce antibodies to RATL1d6 polypeptide (i.e., immunogens) have an amino acid sequence having at least five amino acids, and more preferably, at least 7-10 amino acids. It is also preferable that the immunogens are identical to a portion of the amino acid sequence of the natural protein; they may also contain the entire amino acid sequence of a small, naturally occurring molecule. The peptides, fragments or oligopeptides may comprise a single epitope or antigenic determinant or multiple epitopes. Short stretches of RATL1d6 amino acids may be fused, or covalently attached, to those of another protein, such as KLH, and antibodies are produced against the chimeric molecule.

Monoclonal antibodies to RATL1d6 polypeptide, or immunogenic fragments thereof, may be prepared using any technique which provides for the production of antibody molecules by continuous cell lines in culture. These include, but are not limited to, the hybridoma technique, the human B-cell hybridoma technique, and the EBV-hybridoma technique (G. Kohler et al., 1975, Nature, 256:495-497; D. Kozbor et al., 1985, J. Immunol. Methods, 81:31-42; R. J. Cote et al., 1983, Proc. Natl. Acad. Sci. USA, 80:2026-2030; and S. P. Cole et al., 1984, Mol. Cell Biol., 62:109-120). The production of monoclonal antibodies is well known and routinely used in the art.

In addition, techniques developed for the production of “chimeric antibodies,” the splicing of mouse antibody genes to human antibody genes to obtain a molecule with appropriate antigen specificity and biological activity can be used (S. L. Morrison et al., 1984, Proc. Natl. Acad. Sci. USA, 81:6851-6855; M. S. Neubergeretal., 1984, Nature, 312:604-608; and S. Takeda et al., 1985, Nature, 314:452-454). Alternatively, techniques described for the production of single chain antibodies may be adapted, using methods known in the art, to produce RATL1d6 polypeptide-specific single chain antibodies. Antibodies with related specificity, but of distinct idiotypic composition, may be generated by chain shuffling from random combinatorial immunoglobulin libraries (D. R. Burton, 1991, Proc. Natl. Acad. Sci. USA, 88:11120-3). Antibodies may also be produced by inducing in vivo production in the lymphocyte population or by screening recombinant immunoglobulin libraries or panels of highly specific binding reagents as disclosed in the literature (R. Orlandi et al., 1989, Proc. Natl. Acad. Sci. USA, 86:3833-3837 and G. Winter et al., 1991, Nature, 349:293-299).

Antibody fragments which contain specific binding sites for RATL1d6 polypeptide may also be generated. For example, such fragments include, but are not limited to, F(ab′)₂ fragments which can be produced by pepsin digestion of the antibody molecule and Fab fragments which can be generated by reducing the disulfide bridges of the F(ab′)₂ fragments. Alternatively, Fab expression libraries may be constructed to allow rapid and easy identification of monoclonal Fab fragments with the desired specificity (W. D. Huse et al., 1989, Science, 254.1275-1281).

Various immunoassays can be used for screening to identify antibodies having the desired specificity. Numerous protocols for competitive binding or immunoradiometric assays using either polyclonal or monoclonal antibodies with established specificities are well known in the art. Such immunoassays typically involve measuring the formation of complexes between RATL1d6 polypeptide and its specific antibody. A two-site, monoclonal-based immunoassay utilizing monoclonal antibodies reactive with two non-interfering RATL1d6 polypeptide epitopes is preferred, but a competitive binding assay may also be employed (Maddox, supra).

In an embodiment of the present invention, the polynucleotide encoding RATL1d6 polypeptide, or any fragment or complement thereof, may be used for therapeutic purposes. In one aspect, antisense to the polynucleotide encoding RATL1d6 polypeptide may be used in situations in which it would be desirable to block translation of mRNA, due, at least in some instances, to degradation of mRNA. In particular, cells may be transformed with sequences complementary to polynucleotides encoding RATL1d6 polypeptide. Thus, complementary molecules may be used to modulate RATL1d6 polynucleotide and polypeptide activity, or to achieve regulation of gene function. Such technology is now well known in the art, and sense or antisense oligomers or oligonucleotides, or larger fragments, can be designed from various locations along the coding or control regions of polynucleotide sequences encoding RATL1d6 polypeptide.

Expression vectors derived from retroviruses, adenovirus, herpes or vaccinia viruses, or from various bacterial plasmids may be used for delivery of nucleotide sequences to the targeted organ, tissue or cell population. Methods which are well known to those skilled in the art can be used to construct recombinant vectors which will express nucleic acid sequence that is complementary to the nucleic acid sequence encoding RATL1d6 polypeptide. These techniques are described both in J. Sambrook et al., supra and in F. M. Ausubel et al., supra.

The genes encoding the RATL1d6 polypeptide can be turned off by transforming a cell or tissue with an expression vector that expresses high levels of a RATL1d6 polypeptide-encoding polynucleotide, or a fragment thereof. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector, and even longer if appropriate replication elements are designed to be part of the vector system.

Modifications of gene expression can be obtained by designing antisense molecules or complementary nucleic acid sequences (DNA, RNA, or PNA), to the control, 5′, or regulatory regions of the gene encoding RATL1d6 polypeptide, (e.g., signal sequence, promoters, enhancers, and introns). Oligonucleotides derived from the transcription initiation site, e.g., between positions −10 and +10 from the start site, are preferred. Similarly, inhibition can be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described (See, for example, J. E. Gee et al., 1994, In: B. E. Huber and B. I. Carr, Molecular and Immunologic Approaches, Futura Publishing Co., Mt. Kisco, N.Y.). The antisense molecule or complementary sequence may also be designed to block translation of mRNA by preventing the transcript from binding to ribosomes, or by causing the degradation of the transcripts.

Ribozymes, i.e., enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. Suitable examples include engineered hammerhead motif ribozyme molecules that can specifically and efficiently catalyze endonucleolytic cleavage of sequences encoding RATL1d6 polypeptide.

Specific ribozyme cleavage sites within any potential RNA target are initially identified by scanning the target molecule for ribozyme cleavage sites which include the following sequences: GUA, GUU, and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes according to the invention may be prepared by any method known in the art for the synthesis of nucleic acid molecules. Such methods include techniques for chemically synthesizing oligonucleotides, for example, solid phase phosphoramidite chemical synthesis. Alternatively, RNA molecules may be generated by in vitro and in vivo transcription of DNA sequences encoding RATL1d6. Such DNA sequences may be incorporated into a wide variety of vectors with suitable RNA polymerase promoters such as T7 or SP. Alternatively, the cDNA constructs that constitutively or inducibly synthesize complementary RNA can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl, rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

Many methods for introducing vectors into cells or tissues are available and are equally suitable for use in vivo, in vitro, and ex vivo. For ex vivo therapy, vectors may be introduced into stem cells taken from the patient and clonally propagated for autologous transplant back into that same patient. Delivery by transfection and by liposome injections may be achieved using methods which are well known in the art.

Any of the therapeutic methods described above may be applied to any individual in need of such therapy, including, for example, mammals such as dogs, cats, cows, horses, rabbits, monkeys, and most preferably, humans.

A further embodiment of the present invention embraces the administration of a pharmaceutical composition, in conjunction with a pharmaceutically acceptable carrier, diluent, or excipient, for any of the above-described therapeutic uses and effects. Such pharmaceutical compositions may comprise RATL1d6 nucleic acid, polypeptide, or peptides, antibodies to RATL1d6 polypeptide, mimetics, agonists, antagonists, or inhibitors of RATL1d6 polypeptide or polynucleotide. The compositions may be administered alone or in combination with at least one other agent, such as a stabilizing compound, which may be administered in any sterile, biocompatible pharmaceutical carrier, including, but not limited to, saline, buffered saline, dextrose, and water. The compositions may be administered to a patient alone, or in combination with other agents, drugs, hormones, or biological response modifiers.

The pharmaceutical compositions for use in the present invention can be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, vaginal, or rectal means.

In addition to the active ingredients (i.e., the RATL1d6 nucleic acid or polypeptide, or functional fragments thereof), the pharmaceutical compositions may contain suitable pharmaceutically acceptable carriers or excipients comprising auxiliaries which facilitate processing of the active compounds into preparations which can be used pharmaceutically. Further details on techniques for formulation and administration are provided in the latest edition of Remington's Pharmaceutical Sciences (Maack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained by the combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropyl-methylcellulose, or sodium carboxymethylcellulose; gums, including arabic and tragacanth, and proteins such as gelatin and collagen. If desired, disintegrating or solubilizing agents may be added, such as cross-linked polyvinyl pyrrolidone, agar, alginic acid, or a physiologically acceptable salt thereof, such as sodium alginate.

Dragee cores may be used in conjunction with physiologically suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification, or to characterize the quantity of active compound, i.e., dosage.

Pharmaceutical preparations which can be used orally include push-fit capsules made of gelatin, as well as soft, scaled capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. In addition, suspensions of the active compounds may be prepared as appropriate oily injection suspensions. Suitable lipophilic solvents or vehicles include fatty oils such as sesame oil, or synthetic fatty acid esters, such as ethyloleate or triglycerides, or liposomes. Optionally, the suspension may also contain suitable stabilizers or agents which increase the solubility of the compounds to allow for the preparation of highly concentrated solutions.

For topical or nasal administration, penetrants or permeation agents that are appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes.

The pharmaceutical composition may be provided as a salt and can be formed with many acids, including but not limited to, hydrochloric, sulfuric, acetic, lactic, tartaric, malic, succinic, and the like. Salts tend to be more soluble in aqueous solvents, or other protonic solvents, than are the corresponding free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, combined with a buffer prior to use. After the pharmaceutical compositions have been prepared, they can be placed in an appropriate container and labeled for treatment of an indicated condition. For administration of RATL1d6 product, such labeling would include amount, frequency, and method of administration.

Pharmaceutical compositions suitable for use in the present invention include compositions wherein the active ingredients are contained in an effective amount to achieve the intended purpose. The determination of an effective dose or amount is well within the capability of those skilled in the art. For any compound, the therapeutically effective dose can be estimated initially either in cell culture assays, e.g., using neoplastic cells, or in animal models, usually mice, rabbits, dogs, or pigs. The animal model may also be used to determine the appropriate concentration range and route of administration. Such information can then be used and extrapolated to determine useful doses and routes for administration in humans.

A therapeutically effective dose refers to that amount of active ingredient, for example, RATL1d6 polypeptide, or fragments thereof, antibodies to RATL1d6 polypeptide, agonists, antagonists or inhibitors of RATL1d6 polypeptide, which ameliorates, reduces, or eliminates the symptoms or condition. Therapeutic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio of toxic to therapeutic effects is the therapeutic index, which can be expressed as the ratio, LD₅₀/ED₅₀. Pharmaceutical compositions which exhibit large therapeutic indices are preferred. The data obtained from cell culture assays and animal studies are used in determining a range of dosages for human use. Preferred dosage contained in a pharmaceutical composition is within a range of circulating concentrations that include the ED50 with little or no toxicity. The dosage varies within this range depending upon the dosage form employed, sensitivity of the patient, and the route of administration.

The exact dosage will be determined by the practitioner, who will consider the factors related to the individual requiring treatment. Dosage and administration are adjusted to provide sufficient levels of the active moiety or to maintain the desired effect. Factors which may be taken into account include the severity of the individual's disease state, general health of the patient, age, weight, and gender of the patient, diet, time and frequency of administration, drug combination(s), reaction sensitivities, and tolerance/response to therapy. As a general guide, long-acting pharmaceutical compositions may be administered every 3 to 4 days, every week, or once every two weeks, depending on half-life and clearance rate of the particular formulation.

Normal dosage amounts may vary from 0.1 to 100,000 micrograms (μg), up to a total dose of about 1 gram (g), depending upon the route of administration. Guidance as to particular dosages and methods of delivery is provided in the literature and is generally available to practitioners in the art. Those skilled in the art will employ different formulations for nucleotides than for proteins or their inhibitors. Similarly, delivery of polynucleotides or polypeptides will be specific to particular cells, conditions, locations, and the like.

In another embodiment of the present invention, antibodies which specifically bind to the RATL1d6 polypeptide may be used for the diagnosis of conditions or diseases characterized by expression (or overexpression) of RATL1d6 polynucleotide or polypeptide, or in assays to monitor patients being treated with RATL1d6 polypeptide, or its agonists, antagonists, or inhibitors. The antibodies useful for diagnostic purposes may be prepared in the same manner as those described above for use in therapeutic methods. Diagnostic assays for RATL1d6 polypeptide include methods which utilize the antibody and a label to detect the protein in human body fluids or extracts of cells or tissues. The antibodies may be used with or without modification, and may be labeled by joining them, either covalently or non-covalently, with a reporter molecule. A wide variety of reporter molecules which are known in the art may be used, several of which are described above.

Several assay protocols including ELISA, RIA, and FACS for measuring RATL1d6 polypeptide are known in the art and provide a basis for diagnosing altered or abnormal levels of RATL1d6 polypeptide expression. Normal or standard values for RATL1d6 polypeptide expression are established by combining body fluids or cell extracts taken from normal mammalian subjects, preferably human, with antibody to RATL1d6 polypeptide under conditions suitable for complex formation. The amount of standard complex formation may be quantified by various methods; photometric means are preferred. Quantities of RATL1d6 polypeptide expressed in subject sample, control sample, and disease samples from biopsied tissues are compared with the standard values. Deviation between standard and subject values establishes the parameters for diagnosing disease.

According to another embodiment of the present invention, the polynucleotides encoding RATL1d6 polypeptide may be used for diagnostic purposes. The polynucleotides which may be used include oligonucleotide sequences, complementary RNA and DNA molecules, and PNAs. The polynucleotides may be used to detect and quantify RATL1d6-encoding nucleic acid expression in biopsied tissues in which expression (or under- or overexpression) of RATL1d6 polynucleotide may be correlated with disease. For, example, RATL1d6 polynucleotides, and fragments thereof, may be used to carry out in situ hybridization in both normal and diseased tissues, such as for prognostic, diagnostic, or monitoring purposes, employing labeled RATL1d6 polynucleotide as a probe and techniques known and practiced in the art. RATL1d6 polynucleotide may be radiolabeled, or labeled by other means known in the art, e.g., enzymatic, fluorescent, chemiluminescent, or biotin-avidin systems. The diagnostic assay may be used to distinguish between the absence, presence, and excess expression of RATL1d6, and to monitor regulation of RATL1d6 polynucleotide levels during therapeutic treatment or intervention.

In a related aspect, hybridization with PCR probes which are capable of detecting polynucleotide sequences, including genomic sequences, encoding RATL1d6 polypeptide, or closely related molecules, may be used to identify nucleic acid sequences which encode RATL1d6 polypeptide. The specificity of the probe, whether it is made from a highly specific region, e.g., about 8 to 10 contiguous nucleotides in the 5′ regulatory region, or a less specific region, e.g., especially in the 3′ coding region, and the stringency of the hybridization or amplification (maximal, high, intermediate, or low) will determine whether the probe identifies only naturally occurring sequences encoding RATL1d6 polypeptide, alleles thereof, or related sequences.

Probes may also be used for the detection of related sequences, and should preferably contain at least 50% of the nucleotides encoding RATL1d6 polypeptide. The hybridization probes of this invention may be DNA or RNA and may be derived from the nucleotide sequence of SEQ ID NO:1, or from genomic sequence including promoter, enhancer elements, and introns of the naturally occurring RATL1d6 protein.

Methods for producing specific hybridization probes for DNA encoding RATL1d6 polypeptide include the cloning of nucleic acid sequence that encodes RATL1d6 polypeptide, or RATL1d6 derivatives, into vectors for the production of mRNA probes. Such vectors are known in the art, commercially available, and may be used to synthesize RNA probes in vitro by means of the addition of the appropriate RNA polymerases and the appropriate labeled nucleotides. Hybridization probes may be labeled by a variety of detector/reporter groups, e.g., radionuclides such as ³²P or ³⁵S, or enzymatic labels, such as alkaline phosphatase coupled to the probe via avidin/biotin coupling systems, and the like.

The polynucleotide sequence encoding RATL1d6 polypeptide, or fragments thereof, may be used for the diagnosis of disorders associated with expression of RATL1d6. Examples of such disorders or conditions are described above for “Therapeutics”. The polynucleotide sequence encoding RATL1d6 polypeptide may be used in Southern or Northern analysis, dot blot, or other membrane-based technologies; in PCR technologies; or in dip stick, pin, ELISA or chip assays utilizing fluids or tissues from patient biopsies to detect the status of, e.g., levels or overexpression of RATL1 d6, or to detect altered RATL1d6 expression. Such qualitative or quantitative methods are well known in the art.

In a particular aspect, the nucleotide sequence encoding RATL1 d6 polypeptide may be useful in assays that detect activation or induction of various neoplasms or cancers, particularly those mentioned supra. The nucleotide sequence encoding RATL1d6 polypeptide may be labeled by standard methods, and added to a fluid or tissue sample from a patient under conditions suitable for the formation of hybridization complexes. After a suitable incubation period, the sample is washed and the signal is quantified and compared with a standard value. If the amount of signal in the biopsied or extracted sample is significantly altered from that of a comparable control sample, the nucleotide sequence has hybridized with nucleotide sequence present in the sample, and the presence of altered levels of nucleotide sequence encoding RATL1d6 polypeptide in the sample indicates the presence of the associated disease. Such assays may also be used to evaluate the efficacy of a particular therapeutic treatment regimen in animal studies, in clinical trials, or in monitoring the treatment of an individual patient.

To provide a basis for the diagnosis of disease associated with expression of RATL1d6, a normal or standard profile for expression is established. This may be accomplished by combining body fluids or cell extracts taken from normal subjects, either animal or human, with a sequence, or a fragment thereof, which encodes RATL1d6 polypeptide, under conditions suitable for hybridization or amplification. Standard hybridization may be quantified by comparing the values obtained from normal subjects with those from an experiment where a known amount of a substantially purified polynucleotide is used. Standard values obtained from normal samples may be compared with values obtained from samples from patients who are symptomatic for disease. Deviation between standard and subject (patient) values is used to establish the presence of disease.

Once disease is established and a treatment protocol is initiated, hybridization assays may be repeated on a regular basis to evaluate whether the level of expression in the patient begins to approximate that which is observed in a normal individual. The results obtained from successive assays may be used to show the efficacy of treatment over a period ranging from several days to months.

With respect to cancer, the presence of an abnormal amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier, thereby preventing the development or further progression of the cancer.

Additional diagnostic uses for oligonucleotides designed from the nucleic acid sequence encoding RATL1d6 polypeptide may involve the use of PCR. Such oligomers may be chemically synthesized, generated enzymatically, or produced from a recombinant source. Oligomers will preferably comprise two nucleotide sequences, one with sense orientation (5′→3′) and another with antisense (3′→5′), employed under optimized conditions for identification of a specific gene or condition. The same two oligomers, nested sets of oligomers, or even a degenerate pool of oligomers may be employed under less stringent conditions for detection and/or quantification of closely related DNA or RNA sequences.

Methods suitable for quantifying the expression of RATL1d6 include radiolabeling or biotinylating nucleotides, co-amplification of a control nucleic acid, and standard curves onto which the experimental results are interpolated (P. C. Melby et al., 1993, J. Immunol. Methods, 159:235-244; and C. Duplaa et al., 1993, Anal. Biochem., 229-236). The speed of quantifying multiple samples may be accelerated by running the assay in an ELISA format where the oligomer of interest is presented in various dilutions and a spectrophotometric or colorimetric response gives rapid quantification.

In another embodiment of the present invention, oligonucleotides, or longer fragments derived from the RATL1d6 polynucleotide sequence described herein may be used as targets in a microarray. The microarray can be used to monitor the expression level of large numbers of genes simultaneously (to produce a transcript image), and to identify genetic variants, mutations and polymorphisms. This information may be used to determine gene function, to understand the genetic basis of a disease, to diagnose disease, and to develop and monitor the activities of therapeutic agents. In a particular aspect, the microarray is prepared and used according to the methods described in WO 95/11995 (Chee et al.); D. J. Lockhart et al., 1996, Nature Biotechnology, 14:1675-1680; and M. Schena et al., 1996, Proc. Natl. Acad. Sci. USA, 93:10614-10619). Microarrays are further described in U.S. Pat. No. 6,015,702 to P. Lal et al.

In another embodiment of this invention, the nucleic acid sequence which encodes RATL1d6 polypeptide may also be used to generate hybridization probes which are useful for mapping the naturally occurring genomic sequence. The sequences may be mapped to a particular chromosome, to a specific region of a chromosome, or to artificial chromosome constructions (HACs), yeast artificial chromosomes (YACs), bacterial artificial chromosomes (BACs), bacterial PI constructions, or single chromosome cDNA libraries, as reviewed by C. M. Price, 1993, Blood Rev., 7:127-134 and by B. J. Trask, 1991, Trends Genet., 7:149-154.

Fluorescent In Situ Hybridization (FISH), (as described in 1. Verma et al., 1988, Human Chromosomes: A Manual of Basic Techniques Pergamon Press, New York, N.Y.) may be correlated with other physical chromosome mapping techniques and genetic map data. Examples of genetic map data can be found in numerous scientific journals, or at Online Mendelian Inheritance in Man (OMIM). Correlation between the location of the gene encoding RATL1d6 polypeptide on a physical chromosomal map and a specific disease, or predisposition to a specific disease, may help delimit the region of DNA associated with that genetic disease. The nucleotide sequences, particularly that of SEQ ID NO:1, or fragments thereof, according to this invention may be used to detect differences in gene sequences between normal, carrier, or affected individuals.

In situ hybridization of chromosomal preparations and physical mapping techniques such as linkage analysis using established chromosomal markers may be used for extending genetic maps. Often the placement of a gene on the chromosome of another mammalian species, such as mouse, may reveal associated markers, even if the number or arm of a particular human chromosome is not known. New sequences can be assigned to chromosomal arms, or parts thereof, by physical mapping. This provides valuable information to investigators searching for disease genes using positional cloning or other gene discovery techniques. Once the disease or syndrome has been crudely localized by genetic linkage to a particular genomic region, for example, AT to 11q22-23 (R. A. Gatti et al., 1988, Nature, 336:577-580), any sequences mapping to that area may represent associated or regulatory genes for further investigation. The nucleotide sequence of the present invention may also be used to detect differences in the chromosomal location due to translocation, inversion, and the like, among normal, carrier, or affected individuals.

In another embodiment of the present invention, RATL1d6 polypeptide, its catalytic or immunogenic fragments or oligopeptides thereof, can be used for screening libraries of compounds in any of a variety of drug screening techniques. The fragment employed in such screening may be free in solution, affixed to a solid support, borne on a cell surface, or located intracellularly. The formation of binding complexes, between RATL1d6 polypeptide, or portion thereof, and the agent being tested, may be measured utilizing techniques commonly practiced in the art.

Another technique for drug screening which may be used provides for high throughput screening of compounds having suitable binding affinity to the protein of interest as described in WO 84/03564. In this method, as applied to RATL1d6 protein, large numbers of different small test compounds are synthesized on a solid substrate, such as plastic pins or some other surface. The test compounds are reacted with RATL1d6 polypeptide, or fragments thereof, and washed. Bound RATL1d6 polypeptide is then detected by methods well known in the art. Purified RATL1d6 polypeptide can also be coated directly onto plates for use in the aforementioned drug screening techniques. Alternatively, non-neutralizing antibodies can be used to capture the peptide and immobilize it on a solid support.

The present invention further embraces method for high throughput screening of chemical libraries for agonists or antagonists of RATL1d6. Such assays are based on measuring UBC enzymatic activity, e.g., as described herein in Example 6, or similarly adapted assays which rely on the measurement of UBC activity.

Other screening and small molecule (e.g., drug) detection assays which involve the detection or identification of small molecules that can bind to a given protein, i.e., the RATL1d6 protein, are encompassed by the present invention. Particularly preferred are assays suitable for high throughput screening methodologies. In such binding-based screening or detection assays, a functional assay is not typically required. All that is needed is a target protein, preferably substantially purified, and a library or panel of compounds (e.g., ligands, drugs, small molecules) to be screened or assayed for binding to the protein target. Preferably, most small molecules that bind to the target protein will modulate activity in some manner, due to preferential, higher affinity binding to functional areas or sites on the protein.

An example of such an assay is the fluorescence based thermal shift assay (3-Dimensional Pharmaceuticals, Inc., 3DP, Exton, Pa.) as described in U.S. Pat. Nos. 6,020,141 and 6,036,920 to Pantoliano et al.; see also, J. Zimmerman, 2000, Gen. Eng. News, 20(8)). The assay allows the detection of small molecules (e.g., drugs, ligands) that bind to expressed, and preferably purified, RATL1d6 polypeptide based on affinity of binding determinations by analyzing thermal unfolding curves of protein-drug or ligand complexes. The drugs or binding molecules determined by this technique can be further assayed, if desired, by methods, such as those described herein, to determine if the molecules affect or modulate function or activity of the target protein.

In a further embodiment of this invention, competitive drug screening assays can be used in which neutralizing antibodies capable of binding RATL1d6 polypeptide specifically compete with a test compound for binding to RATL1d6 polypeptide. In this manner, the antibodies can be used to detect the presence of any peptide which shares one or more antigenic determinants with RATL1d6 polypeptide.

It will be understood that the nucleotide sequences which encode RATL1d6 polypeptide may be used in any molecular biology techniques that have yet to be developed, provided the new techniques rely on properties of nucleotide sequences that are currently known, including, but not limited to, such properties as the triplet genetic code and specific base pair interactions. Motifs and Descriptions

The RATL1d6 polypeptide of the present invention was determined to comprise several phosphorylation sites based upon the Motif algorithm (Genetics Computer Group, Inc.). The phosphorylation of such sites may regulate biological activity of the RATL1d6 polypeptide. For example, phosphorylation at specific sites may be involved in regulating the ability of the protein to associate or bind to other molecules (e.g., proteins, ligands, substrates, DNA, etc.). In the present case, phosphorylation may modulate the ability of the RATL1d6 polypeptide to associate with other polypeptides, particularly a cognate ligand for RATL1d6, or its ability to modulate certain cellular signal pathways.

Specifically, the RATL1d6 polypeptide was predicted to comprise four protein kinase C (PKC) phosphorylation sites using the Motif algorithm (Genetics Computer Group, Inc.). In vivo, PKC exhibits a preference for the phosphorylation of serine or threonine residues. The PKC phosphorylation sites have the following consensus pattern: [ST]-x-[RK], where S or T represents the site of phosphorylation and ‘x’ an intervening amino acid residue. Additional information regarding PKC phosphorylation sites can be found in Woodget, J. R. et al., 1986, Eur. J. Biochem., 161:177-184 and Kishimoto A. et al., 1985, J. Biol. Chem., 260:12492-12499, which are hereby incorporated by reference herein.

Preferably, the following PKC phosphorylation site polypeptides are encompassed by the present invention: GSVQATDRLMKEL (SEQ ID NO:18), IYRSQSFKGGNYA (SEQ ID NO:19), ILLNFSFKDNFPF (SEQ ID NO:20), and/or TRAQQSYKSLVQI (SEQ ID NO:21). Polynucleotides encoding these polypeptides are also provided. The present invention also encompasses the use of one or more of the RATL1d6 PKC phosphorylation site polypeptides as immunogenic and/or antigenic epitopes as described elsewhere herein.

The RATL1d6 polypeptide was predicted to comprise six casein kinase II phosphorylation sites using the Motif algorithm (Genetics Computer Group, Inc.). Casein kinase II (CK-2) is a protein serine/threonine kinase whose activity is independent of cyclic nucleotides and calcium. CK-2 has the ability to phosphorylate many different proteins. The substrate specificity of this enzyme can be summarized as follows: (1) Under comparable conditions Ser is favored over Thr; (2) An acidic residue (either Asp or Glu) must be present three residues from the C-terminus of the phosphate acceptor site; (3) Additional acidic residues in positions +1, +2, +4 and +5 increase the phosphorylation rate. Most physiological substrates have at least one acidic residue in these positions; (4) Asp is preferred over Glu as the provider of acidic determinants; and (5) A basic residue at the N-terminus of the acceptor site decreases the phosphorylation rate, while an acidic residue increases it.

A consensus pattern for a typical casein kinase 11 phosphorylation site is as follows: [ST]-x(2)-[DE], where ‘x’ represents any amino acid, and S or T is the phosphorylation site. Additional information specific to aminoacyl-transfer RNA synthetase class-II domains can be found in the following publication: Pinna, L. A., 1990, Biochim. Biophys. Acta, 1054:267-284; which is hereby incorporated by reference herein in its entirety.

The following casein kinase 11 phosphorylation site polypeptides are preferably encompassed by the present invention: PAEQCTQEDVSSED (SEQ ID NO:22), TQEDVSSEDEDEEM (SEQ ID NO:23), QEDVSSEDEDEEMP (SEQ ID NO:24), AEGKKSEDDGIGKE (SEQ ID NO:25), ELVNDSLYDWNVKL (SEQ ID NO:26), and/or ILLNFSFKDNFPFD (SEQ ID NO:27). Polynucleotides encoding these polypeptides are also provided. The present invention also encompasses the use of the casein kinase II phosphorylation site polypeptides as an immunogenic and/or antigenic epitope as described elsewhere herein.

The RATL1d6 polypeptide has been shown to comprise four glycosylation sites according to the Motif algorithm (Genetics Computer Group, Inc.). As discussed more specifically herein, protein glycosylation is thought to serve a variety of functions including augmentation of protein folding, inhibition of protein aggregation, regulation of intracellular trafficking to organelles, increasing resistance to proteolysis, modulation of protein antigenicity, and mediation of intercellular adhesion.

Asparagine glycosylation sites have the following consensus pattern, N-{P}-[ST]-{P}, where N represents the glycosylation site. It is well known that potential N-glycosylation sites are specific to the consensus sequence Asn-Xaa-Ser/Thr. However, the presence of the consensus tripeptide is not sufficient to conclude that an asparagine residue is glycosylated, due to the fact that the folding of the protein plays an important role in the regulation of N-glycosylation. It has been shown that the presence of proline between Asn and Ser/Thr will inhibit N-glycosylation; this has been confirmed by a recent statistical analysis of glycosylation sites, which also shows that about 50% of the sites that have a proline C-terminal to Ser/Thr are not glycosylated. Additional information relating to asparagine glycosylation can be found in the following publications, which are hereby incorporated by reference herein: Marshall R. D., Annu. Rev. Biochem., 41:673-702(1972); Pless D. D. and Lennarz W. J., Proc. Natl. Acad. Sci. U.S.A., 74:134-138(1977); Bause E., Biochem. J., 209:331-336(1983); Gavel Y. and von Heijne G., Protein Eng., 3:433-442(1990); and Miletich J. P. and Broze G. J. Jr., J. Biol. Chem., 265:11397-11404(1990).

In preferred embodiments, the following asparagine glycosylation site polypeptides are encompassed by the present invention: VRIHCNITESYPAV (SEQ ID NO:28), AVELVNDSLYDWNV (SEQ ID NO:29), DFILLNFSFKDNFP (SEQ ID NO:30), and/or VQFGANKSQYSLTR (SEQ ID NO:31). Polynucleotides encoding these polypeptides are also provided. The present invention also encompasses the use of one or more of these RATL1d6 asparagine glycosylation site polypeptides as immunogenic and/or antigenic epitopes as described elsewhere herein.

The RATL1d6 polypeptide was predicted to comprise fourteen N-myristylation sites using the Motif algorithm (Genetics Computer Group, Inc.). An appreciable number of eukaryotic proteins are acylated by the covalent addition of myristate (a C₁₄-saturated fatty acid) to their N-terminal residue via an amide linkage. The sequence specificity of the enzyme responsible for this modification, myristyl CoA:protein N-myristyl transferase (NMT), has been derived from the sequence of known N-myristylated proteins and from studies using synthetic peptides. The specificity seems to be the following: i) The N-terminal residue must be glycine; ii) In position 2, uncharged residues are allowed; iii) Charged residues, proline and large hydrophobic residues are not allowed; iv) In positions 3 and 4, most, if not all, residues are allowed; v) In position 5, small uncharged residues are allowed (Ala, Ser, Thr, Cys, Asn and Gly). Serine is favored; and vi) In position 6, proline is not allowed.

A consensus pattern for N-myristylation is as follows: G-{EDRKHPFYW}-x(2)-[STAGCN]-{P}, wherein ‘x’ represents any amino acid, and G is the N-myristylation site. Additional information specific to N-myristylation sites may be found in the following publications: Towler D. A. et al., Annu. Rev. Biochem., 57:69-99(1988); and Grand R. J. A., Biochem. J., 258:625-638 (1989); which are hereby incorporated by reference herein in their entirety.

The following N-myristylation site polypeptides are preferably encompassed by the present invention: QQPGPGQQLGGQGMP (SEQ ID NO:32), PGQQLGGQGAAPGAGG (SEQ ID NO:33), QLGGQGMPGAGGGPG (SEQ ID NO:34), AAPGAGGGPGGGPGPG (SEQ ID NO:35), APGAGGGPGGGPGPGP (SEQ ID NO:36), EFLLAGAGGAGAGAAP (SEQ ID NO:37), LLAGAGGAGAGMPGP (SEQ ID NO:38), LAGAGGAGAGAAPGPH (SEQ ID NO:39), HLPPRGSVPGDPVRIH (SEQ ID NO:40), QDYLNGAVSGSVQATD (SEQ ID NO:41), NGAVSGSVQATDRLMK (SEQ ID NO:42), SQSFKGGNYAVELVND (SEQ ID NO:43), GYVLGGGAICMELLTK (SEQ ID NO:44), and/or ARVQFGANKSQYSLTR (SEQ ID NO:45). Polynucleotides encoding these polypeptides are also provided. The present invention further encompasses the use of these RATL1 d6 N-myristylation site polypeptides as immunogenic and/or antigenic epitopes as described elsewhere herein.

The RATL1d6 polypeptide has been shown to comprise one amidation site according to the Motif algorithm (Genetics Computer Group, Inc.). The precursor of hormones and other active peptides which are C-terminally amidated is always directly followed by a glycine residue which provides the amide group, and most often by at least two consecutive basic residues (Arg or Lys) which generally function as an active peptide precursor cleavage site. Although all amino acids can be amidated, neutral hydrophobic residues, such as Val or Phe, are good substrates, while charged residues, such as Asp or Arg, are much less reactive. A consensus pattern for amidation sites is the following: x-G-[RK]-[RK], wherein “x” represents the amidation site. Additional information relating to amidation may be found in the following publications, which are hereby incorporated by reference herein: Kreil, G., Meth. Enzymol., 106:218-223(1984) and Bradbury, A. F. and Smyth D. G., Biosci. Rep., 7:907-916(1987).

In a preferred embodiment, the following amidation site polypeptide is encompassed by the present invention: EEEPAEGKKSEDDG (SEQ ID NO:46). The polynucleotide encoding this polypeptide is also provided. The present invention also encompasses the use of this RATL1d6 amidation site polypeptide as an immunogenic and/or antigenic epitope as described elsewhere herein.

Enhancing the Biological Activity/Functional Characteristics of the Present Invention Through Molecular Evolution

Although many of the most biologically active proteins known are highly effective for their specified function in an organism, they often possess characteristics that make them undesirable for transgenic, therapeutic, pharmaceutical and/or industrial applications. Among these traits, a short physiological half-life is the most prominent problem, and is present either at the level of the protein, or at the level of the mRNA. The ability to extend the half-life of a protein or peptide would be particularly important for its use, for example, in gene therapy, transgenic animal production, the bioprocess production and purification of the protein and the use of the protein as a chemical modulator, among others. Therefore, there is a need to identify novel variants of isolated proteins possessing characteristics which enhance their application as therapeutics for treating diseases of animal origin, in addition to having applicability to common industrial and pharmaceutical applications.

Thus, one aspect of the present invention relates to the ability to enhance specific characteristics of the polypeptides of the present invention through directed molecular evolution. Such an enhancement may, in a non-limiting example, benefit the utility of the newly described polynucleotide and/or protein products as an essential component in a kit; the physical attributes of a protein and/or polynucleotide of the invention, such as its solubility, structure, or codon optimization; the specific biological activity of a protein of the invention, including any associated enzymatic activity; the enzyme kinetics of the proteins of the invention (if applicable); the Ki, Kcat, Km, Vmax, Kd, protein-protein activity, protein-DNA binding activity, antagonist/inhibitory activity (including direct or indirect interaction), agonist activity (including direct or indirect interaction) of the protein of the invention; the antigenicity of the protein of the invention (e.g., where it would be desirable to either increase or decrease the antigenic potential of the protein); the immunogenicity of the protein of the invention; the ability of the protein of the invention to form dimers, trimers, or multimers with either itself or other proteins; the antigenic efficacy of a protein of the invention, including its subsequent use as a preventative treatment for disease or disease states, or as an effector for targeting diseased genes.

Moreover, the ability to enhance specific characteristics of a protein may also be applicable to changing the characterized activity of an enzyme to an activity completely unrelated to its initially characterized activity. Other desirable enhancements of the protein of the present invention would be specific to each individual protein, and would thus be appreciated by the skilled practitioner in the art and contemplated by the present invention.

For example, an engineered ubiquitin conjugating enzyme, e.g., RATL1d6 protein, may be constitutively active upon binding of its substrate. Alternatively, an engineered ubiquitin conjugating enzyme may be constitutively active in the absence of substrate binding. In yet another example, an engineered ubiquitin conjugating enzyme may be capable of being activated with less than all of the regulatory factors and/or conditions typically required for ubiquitin conjugating enzyme activation (e.g., substrate binding, phosphorylation, conformational changes, etc.). Such a ubiquitin conjugating enzyme would be useful in screens to identify ubiquitin conjugating enzyme modulators, among other uses described herein. Alternatively, an engineered ubiquitin conjugating enzyme may have altered substrate specificity, and/or enhanced ubiquitin conjugating enzyme activity. As yet another alternative, an engineered ubiquitin conjugating enzyme may have decreased ubiquitin conjugating enzyme activity.

Directed evolution is comprised of several steps. The first step involves establishing a library of variants for the gene or protein of interest. The most important step is then selecting for those variants which possess the activity to be identified. The design of the screen is essential, since the screen should be selective enough to eliminate non-useful variants, but not so stringent as to eliminate all variants. The last step is repeating the above steps using the best variant from the previous screen. Each successive cycle can then be tailored as necessary, such as by increasing the stringency of the screen, for example.

Over the years, there have been a number of methods developed to introduce mutations into macromolecules. Some of these methods include random mutagenesis, “error-prone” PCR, chemical mutagenesis, site-directed mutagenesis, and other methods well known in the art (for a comprehensive listing of current mutagenesis methods, see T. Maniatis et al., Molecular Cloning: A Laboratory Manual, Cold Spring Harbor Press, Cold Spring, N.Y. (1982)). Typically, such methods have been used, for example, as tools for identifying the core functional region(s) of a protein or the function of specific domains of a protein (if a multi-domain protein). However, such methods have more recently been applied to the identification of macromolecule variants with specific or enhanced characteristics.

Random mutagenesis has been the most widely recognized method to date. Typically, this has been carried out either through the use of “error-prone” PCR (as described in Moore, J. et al, Nature Biotechnology 14:458, (1996), or through the application of randomized synthetic oligonucleotides corresponding to specific regions of interest (as described by Derbyshire, K. M. et al, Gene, 46:145-152, (1986), and Hill, D. E. et al, Methods Enzymol., 55:559-568, (1987)). Both approaches have limits to the level of mutagenesis that can be obtained. However, either approach enables the investigator to effectively control the rate of mutagenesis. This is particularly important, since mutations beneficial to the activity of the enzyme are fairly rare. In fact, using too high a level of mutagenesis may counter or inhibit the desired benefit of a useful mutation.

While both of the aforementioned methods are effective for creating randomized pools of macromolecule variants, a third method, termed “DNA Shuffling”, or “sexual PCR” (Stemmer, W. P. C., PNAS, 91:10747, (1994)) has recently been elucidated. DNA shuffling has also been referred to as “directed molecular evolution”, “exon-shuffling”, “directed enzyme evolution”, “in vitro evolution”, and “artificial evolution”. Such reference terms are known in the art and are encompassed by the invention. The new, preferred, method apparently overcomes the limitations of the previous methods in that it not only propagates positive traits, but simultaneously eliminates negative traits in the resulting progeny.

DNA shuffling accomplishes this task by combining the principal of in vitro recombination, along with the method of “error-prone” PCR. In effect, a randomly digested pool of small DNA fragments of a given gene (i.e., a RATL1d6 gene according to this invention) is created by DNase I digestion. The resulting fragments are then introduced into an “error-prone” PCR assembly reaction. During the PCR reaction, the randomly sized DNA fragments hybridize not only to their cognate strand, but also to other DNA fragments corresponding to different regions of the polynucleotide of interest—regions not typically accessible via hybridization of the entire polynucleotide. Moreover, since the PCR assembly reaction utilizes “error-prone” PCR reaction conditions, random mutations are introduced during the DNA synthesis step of the PCR reaction for all of the fragments, thus further diversifying the potential hybridization sites during the annealing step of the reaction.

A variety of reaction conditions can be employed to carry out the DNA shuffling reaction. However, specific reaction conditions for DNA shuffling are provided hereinbelow for guidance, (see also, PNAS, 91:10747, (1994). Briefly: the DNA substrate that is to be subjected to the DNA shuffling reaction is prepared. The preparation may be in the form of simply purifying the DNA from contaminating cellular material, chemicals, buffers, oligonucleotide primers, deoxynucleotides, RNAs, etc., and may utilize commercially available DNA purification kits, such as those provided by Qiagen, Inc., or by Promega, Corp., for example.

Once the DNA substrate has been purified, it is subjected to DNase I digestion. About 2-4 μg of the DNA substrate(s) is digested with 0.0015 units of DNase I (Sigma) per μl in 100 μl of 50 mM Tris-HCL, pH 7.4/1 mM MgCl₂ for 10-20 minutes at room temperature. The resulting fragments of 10-50 bp are then purified by subjecting them to agarose gel electrophoresis (e.g., a 2% low-melting point agarose gel) and then transferring them onto DE81 ion-exchange paper (Whatman); the fragment can also be purified using Microcon concentrators (Amicon) of the appropriate molecular weight cutoff, or by using oligonucleotide purification columns (Qiagen), in addition to other methods known in the art. If using DE81 ion-exchange paper, the 10-50 bp fragments are then eluted from said paper using 1 M NaCl, followed by ethanol precipitation.

The resulting purified fragments are then be subjected to a PCR assembly reaction by re-suspension in a PCR mixture containing: 2 mM of each dNTP, 2.2 mM MgCl₂, 50 mM KCl, 10 mM Tris-HCL, pH 9.0, and 0.1% Triton X-100®, at a final fragment concentration of 10-30 ng/μl. No primers are added at this point.

Taq DNA polymerase (Promega) is used at 2.5 units per 100 μl of reaction mixture. A PCR program used is 94 C for 60 s; 94 C for 30 sec, 50-55 C for 30 sec, and 72 C for 30 sec using 30-45 cycles, followed by 72 C for 5 min using an MJ Research (Cambridge, Mass.) PTC-150 thermocycler. After the assembly reaction is completed, a 1:40 dilution of the resulting primeness product is then introduced into a PCR mixture (using the same buffer mixture used for the assembly reaction) containing 0.8 μm of each primer and subjecting this mixture to 15 cycles of PCR (using 94 C for 30 s, 50 C for 30 s, and 72 C for 30 s). The referred primers are primers corresponding to the nucleic acid sequences of the polynucleotide(s) utilized in the shuffling reaction. Such primers can contain modified nucleic acid base pairs using methods known in the art and referred to elsewhere herein, or can contain additional sequences (i.e., for adding restriction sites, mutating specific base pairs, etc.).

The resulting shuffled, assembled, and amplified product can be purified using methods well known in the art (e.g., Qiagen PCR purification kits) and then subsequently cloned using appropriate restriction enzymes.

Although a number of variations of DNA shuffling have been published to date, such variations are understood and practiced by the skilled artisan and are encompassed by the invention. The DNA shuffling method can also be tailored to the desired level of mutagenesis using the methods described by Zhao et al. (Nucl Acid Res., 25(6):1307-1308, (1997)).

As described above, once the randomized pool has been created, it can then be subjected to a specific screen to identify the variant possessing the desired characteristic(s). Once the variant has been identified, DNA corresponding to the variant can be used as the DNA substrate for initiating another round of DNA shuffling. This cycle of shuffling, selecting the optimized variant of interest, and then re-shuffling, can be repeated until the ultimate variant is obtained. Examples of model screens applied to identify variants created using DNA shuffling technology are found in the following publications: J. C. Moore et al., J. Mol. Biol, 272:336-347, (1997), F. R. Cross et al., Mol. Cell. Biol., 18:2923-2931, (1998), and A. Crameri et al., Nat. Biotech., 15:436-438, (1997).

DNA shuffling has several advantages. First, it makes use of beneficial mutations. When combined with screening, DNA shuffling allows the discovery of the best mutational combinations and does not assume that the best combination contains all the mutations in a population. Second, recombination occurs simultaneously with point mutagenesis. An effect of forcing DNA polymerase to synthesize full-length genes from the small fragment DNA pool is a background mutagenesis rate. In combination with a stringent selection method, enzymatic activity has been evolved to up to a 16000-fold increase over the wild-type form of the enzyme. In essence, the background mutagenesis yielded the genetic variability on which recombination acted to enhance the activity.

A third feature of recombination is that it can be used to remove deleterious mutations. As discussed above, during the process of the randomization, for every one beneficial mutation, there may be at least one or more neutral or inhibitory mutation(s). Such mutation(s) can be removed by including in the assembly reaction an excess of the wild-type random-size fragments, in addition to the random-size fragments of the selected mutant from the previous selection. During the subsequent selection, some of the most active variants of the polynucleotide/polypeptide/enzyme should have lost the inhibitory mutations.

Finally, recombination enables parallel processing. This represents a significant advantage, since there are likely to be multiple characteristics that would make a protein more desirable (e.g. solubility, activity, etc.). Since it is increasingly difficult to screen for more than one desirable trait at a time, other methods of molecular evolution tend to be inhibitory. However, using recombination, it is possible to combine the randomized fragments of the best representative variants for the various traits, and then to select for multiple properties at one time. [0261] DNA shuffling can also be applied to the polynucleotides and polypeptides of the present invention to decrease their immunogenicity in a specified host, particularly if the polynucleotides and polypeptides provide a therapeutic use. For example, a particular variant of the present invention may be created and isolated using DNA shuffling technology. Such a variant may have all of the desired characteristics, though it may be highly immunogenic in a host due to its novel intrinsic structure. Specifically, the desired characteristic may cause the polypeptide to have a non-native structure which is no longer be recognized as a “self” molecule, but rather as a “foreign” molecule, and thus activate a host's immune response directed against the novel variant. Such a limitation can be overcome, for example, by including a copy of the gene sequence for a xenobiotic ortholog of the native protein with the gene sequence of the novel variant gene in one or more cycles of DNA shuffling. The molar ratio of the ortholog and novel variant DNAs could be varied accordingly. Ideally, the resulting hybrid variant identified would contain at least some of the coding sequence which enabled the xenobiotic protein to evade the host immune system, and additionally, the coding sequence of the original novel variant that provided the desired characteristics.

Likewise, the present invention encompasses the application of DNA shuffling technology to the evolution of polynucleotides and polypeptides of the invention, wherein one or more cycles of DNA shuffling include, in addition to the gene template DNA, oligonucleotides coding for known allelic sequences, optimized codon sequences, known variant sequences, known polynucleotide polymorphism sequences, known ortholog sequences, known homolog sequences, additional homologous sequences, additional non-homologous sequences, sequences from another species, and any number and combination of the above.

In addition to the above-described methods, there are a number of related methods that may also be applicable, or desirable, in certain cases. Representative among these are the methods discussed in PCT applications WO 98/31700, and WO 98/32845, which are hereby incorporated by reference. Furthermore, related methods can also be applied to the polynucleotide sequences of the present invention in order to evolve and create ideal variants for use in gene therapy, protein engineering, evolution of whole cells containing the variant, or in the evolution of entire enzyme pathways, containing polynucleotides of the invention, such as described in PCT applications WO 98/13485, WO 98/13487, WO 98/27230, WO 98/31837, and Crameri, A. et al., Nat. Biotechnol., 15:436-438, (1997).

Additional methods of applying “DNA Shuffling” technology to the polynucleotides and polypeptides of the present invention, including their proposed applications, may be found in U.S. Pat. No. 5,605,793; PCT Application No. WO 95/22625; PCT Application No. WO 97/20078; PCT Application No. WO 97/35966; and PCT Application No. WO 98/42832. The foregoing are hereby incorporated by reference in their entirety herein for all purposes.

### EXAMPLES

The Examples below are provided to illustrate the subject invention and are not intended to limit the invention.

### Example 1

### Methods

A. T-Cell Preparation

T cells were prepared by standard rosetting protocols with sheep red blood cells (SRBCs). Briefly, peripheral blood mononuclear cells (PBMCs) from 225 ml of heparinized blood from each of 2 donors were prepared by centrifugation over Ficoll. T cells (E+fraction) were isolated by rosetting with SRBCs. Messenger RNA (mRNA) from one half of the unstimulated T cells (approximately 2.25×10⁸ cells) was prepared according to the manufacturer's instructions with a FAST-TRACK mRNA isolation kit (Invitrogen). The remaining T cells were diluted to 1.25×10⁶/ml in RPMI/10%FBS containing the costimulatory anti-CD28 mAb 2E12 at 5 μg/ml and added (20 ml/plate) to 10 cm tissue culture plates (Corning) that had been coated with anti-CD3 mAb G19-4. Plates were coated by incubating 5 ml of 5 ug/ml mAb diluted in PBS for 7hours at 37° C. followed by washing 3 times with PBS. The plates were cultured under normal conditions of cell culture for 18hours. Activated cells were harvested by vigorous pipetting and scraping to obtain both suspension and adherent cells, pelleted by centrifugation, and processed for mRNA isolation as described above.

B. Subtraction Library Construction

A cDNA subtraction library was made using the CLONTECH PCR-Select™ cDNA Subtraction Kit (Clontech, Palo Alto, Calif.). Manufacturer's protocols were followed for 500 ng of anti-CD3/anti-CD28 activated peripheral blood T cell poly A+ RNA (tester) and 500 ng of unactivated, resting peripheral blood T cell poly A+ RNA (driver). Five secondary PCR reactions were combined and run on a 1.2% agarose gel. Fragments ranging from approximately 0.3 kb-1.5 kb were gel purified using the QIAgen gel extraction kit (QIAgen Inc., Valencia, Calif.) and inserted into the TA cloning vector, pCR2.1 (Invitrogen). TOP1° F. competent E. Coli (Invitrogen) were transformed and plated on Lauria-Bertani (LB) plates containing 50 micrograms/ml ampicillin. Approximately 600 clones were isolated and grown in LB broth containing similar concentrations of ampicillin. Plasmids were isolated using QlAgen miniprep spin (QlAgen) and sequenced using ABI cycle sequencers (ABI Prism, PE Applied Biosystems).

C. Database Mining for Overlapping EST Clones

Over six hundred clone inserts were analyzed using BLAST2 (Basic Local Alignment Search Tool). Clones with ESTs (Expressed Sequence Tags) of known genes were removed using the non-redundant nucleotide database maintained by NCBI. A number of clones proved to be novel, i.e., such clones were not published by NCBI or found in the database. A further search was performed using the geneseq nucleotide patent database (also available through BLAST2). Clones containing ESTs of patented sequences were eliminated depending on the size of the known sequence, quality of known sequence information, and/or lack of utility associated with publicly available sequence.

After analyzing the sequences for novelty, virtual cloning was performed using the D2 clustered EST database (also available on BLAST2). This D2 clustered database was designed by the Bristol-Myers Squibb Bioinformatics department. It contains both public and proprietary (Incyte Pharmaceuticals, Inc.) ESTs assembled into contigs. A contig is a collection of smaller EST clones assembled into larger sequence fragments. The subtraction clone sequences were used to query this clustered database. Cluster sequences were assembled with the subtraction clone sequence using the sequence analysis program Sequencher (Gene Codes). The larger contig sequence was then back-searched against the non-redundant nucleotide (NRN), geneseq nucleotide patent (GNP), non-redundant protein (NRP), and geneseq peptide patent (GPP) databases.

D. Drosophila Ortholog Identification

To search the Drosophila orthologue of the human RATL1d6 gene, the RATL1d6 protein sequence was searched against the public Drosophila protein and genomic sequence database from GenBank, using the BLAST software (Altschul, S. F. et al., 1997, Nucleic Acids Res., 25:3389-3402). The Drosophila gene EG:25E8 (Genbank Accession No: AAF45767) was found to have the highest homology with the human RATL1 d6 gene, with 48% identity at the amino acid level covering the majority of the gene. The Drosophila gene EG:25E8 was used to search against the public human protein and genomic sequence database from GenBank. Among all of the human genes, RATL1d6 was found to be most similar to the Drosophila EG:25E8 gene. The results of the database search indicate that EG:25E8 is the Drosophila orthologue of the human UBC enzyme RATL1d6 gene.

### Example 2

### Cloning of RATL1d6 Polynucleotide

Full-length cloning experiments to isolate and obtain the RATL1d6 polynucleotide were performed using Gene Trapper technology (LifeTechnologies, Md.) according to the manufacturer's instructions. Briefly, PCR primers PY508, (5′-TGCAGTGTCTGGCTCGGTGC-3′), (SEQ ID NO:9) and PY509, (5′-CTGATCTGCATGATCACTGAC-3′), (SEQ ID NO:10) were used to screen a panel of human cDNA libraries (LifeTechnologies), including bone marrow, heart, lung, brain, kidney, peripheral blood leukocyte, liver, spleen, testis and fetal brain cDNA libraries. A strong positive PCR product was identified in human brain and bone marrow cDNA libraries (LifeTechnologies).

The double stranded cDNA plasmid libraries were converted to single stranded DNA (ssDNA) using Gene II and Exonuclease II. Hybrids between the biotinylated oligonucleotide (PY495: 5′-TCCACTGCMCATCACGGAGTCATACCCTG-3′), (SEQ ID NO: 11) or (PY496: 5′-ATGCAGTCGAACTCGTGAATGACAGTCTGT-3′), (SEQ ID NO:12) and the ssDNA were formed and then captured on paramagnetic beads. ((D. A. Tagle et al., 1993, Nature, 361:751-753). After washing, the ssDNA was released and converted to dsDNA by DNA polymerase. Following transformation and plating of DH10B cells, positive clones were identified by PCR analysis. Through this technique, positive clones were identified for the novel RATL1 d6 gene. The plasmids were prepared using a QIAprep spin miniprep kit (Qiagen) and the resulting DNA was subjected to sequencing using conventional protocols known in the art.

Sequence analysis of the 5′ end of the sequenced polynucleotide indicated that three of the clones from the bone marrow cDNA library contained the full-length coding region for the RATL1d6 polypeptide. Additional primers were synthesized and used to sequence the entire insert using conventional sequencing protocols. The vector for these cDNA inserts was pCMVSPORT2 with cloning sites SalI (5′-end) and NotI (3′-end).

### Example 3

### Labeling of Hybridization Probes and Use Thereof

Hybridization probes derived from SEQ ID NO:1 are employed to screen cDNAs, genomic DNAs, or mRNAs. Although the labeling of oligonucleotides containing about 20 base pairs is described in this Example, essentially the same procedure is used with larger cDNA fragments. Oligonucleotides are designed using state-of-the-art software such as OLIGO 4.06 (National Biosciences), labeled by combining 50 pmol of each oligomer and 250 μCi of [γ-³²P] adenosine triphosphate (Amersham) and T4 polynucleotide kinase (DuPont NEN, Boston, Mass.). The labeled oligonucleotides are substantially purified with SEPHADEX G-25 superfine resin column (Amersham Pharmacia Biotech). A portion containing 10⁷ counts per minute of each of the sense and antisense oligonucleotides is used in a typical membrane based hybridization analysis of human genomic DNA digested with one of the following endonucleases (e.g., Ase I, Bgl II, Eco RI, Pst I, Xba 1, or Pvu II, DuPont NEN).

The DNA from each digest is fractionated on a 0.7 percent agarose gel and transferred to nylon membranes (Nytran Plus, Schleicher & Schuell, Durham, N.H.). Hybridization is carried out for 16 hours at 40° C. To remove nonspecific signals, blots are sequentially washed at room temperature under increasingly stringent conditions up to 0.1×saline sodium citrate and 0.5% sodium dodecyl sulfate. After XOMATAR film (Kodak, Rochester, N.Y.) is exposed to the blots in a Phosphoimager cassette (Molecular Dynamics, Sunnyvale, Calif.) for several hours, hybridization patterns are compared visually.

### Example 4

### Complementary Polynucleotides

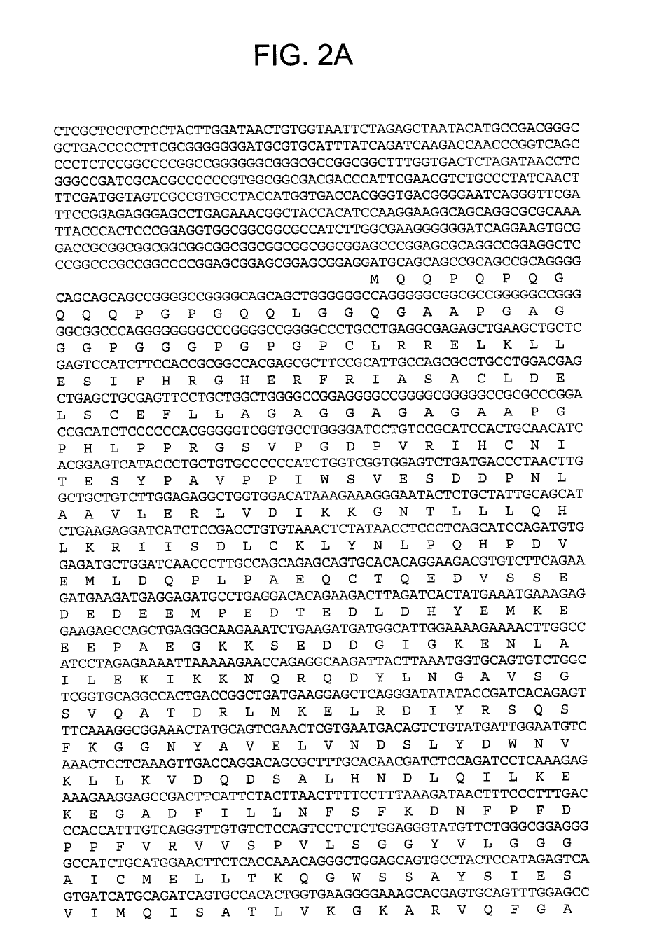

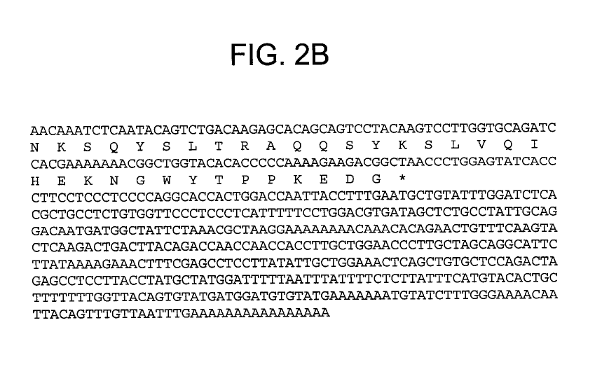

Antisense molecules or nucleic acid sequence complementary to the RATL1d6 protein-encoding sequence, or any part thereof, is used to decrease or to inhibit the expression of naturally occurring RATL1d6. Although the use of antisense or complementary oligonucleotides comprising about 15 to 35 base-pairs is described, essentially the same procedure is used with smaller or larger nucleic acid sequence fragments. An oligonucleotide based on the coding sequence of RATL1d6 protein, as shown in FIGS. 1A, 1B and 2A, 2B, is used to inhibit expression of naturally occurring RATL1d6. The complementary oligonucleotide is designed from the most unique 5′ sequence (FIGS. 1A, 1B and 2A, 2B), and is used either to inhibit transcription by preventing promoter binding to the coding sequence, or to inhibit translation by preventing the ribosome from binding to the RATL1d6 protein-encoding transcript. Using an appropriate portion of the signal and 5′ sequence of SEQ ID NO:1, an effective antisense oligonucleotide includes any of about 15-35 nucleotides to target any portion of the mRNA, preferably, an antisense oligo spans the region which translates into the signal, or the coding sequence of the polypeptide as shown in FIGS. 1A, 1B and 2A, 2B. Appropriate oligonucleotides are designed using OLIGO 4.06 software and RATL1d6 protein coding sequence.

### Example 5

### Expression of RATL1d6

Expression of RATL1d6 polypeptide is achieved by subcloning the encoding cDNA into appropriate vectors and transforming the vectors into host cells. For this example, the cloning vector, pGEX, is used to express RATL1 d6 in DH5α host cells.

By way of this Example, using E. coli as the host cell, the cloning vector contains a promoter for β-galactosidase upstream of the cloning site, followed by sequence encoding glutathione S-transferase (GST). Immediately following these residues is a bacteriophage promoter that is useful for transcription and a linker containing a number of unique restriction sites.

Induction of an isolated transformed bacterial strain with IPTG using standard methods produces a fusion protein which contains the first eight residues of β-galactosidase, about 5-15 residues of linker, GST and then full-length RATL1d6 protein. The signal residues direct the secretion of RATL1d6 protein into the bacterial growth medium, which can be used directly in the assays to determine the activity of the protein.

### Example 6

### Demonstration of RATL1d6 Activity

RATL1d6 gene product activity is demonstrated in vitro by the formation of multi-ubiquitin conjugates and ubiquitin-RATL1d6 thiol ester linkage from free ubiquitin and E2 ubiquitin carrier protein, which catalyzes multi-ubiquitin chain formation (S. Van Nocker and R. D. Vierstra, 1991, Proc. Nat'l. Acad. Sci. USA, 88:10297-10301). Briefly, thiol ester adduct formation between ubiquitin and E2 (20 kDa), control, and RATL1d6 protein is assayed as described by A. L. Haas et al., 1982, J. Biol. Chem., 257:2543-2548. ¹²⁵1-labeled ubiquitin or [Arg⁴⁸] ubiquitin (1 μM) is incubated in a reaction mixture with RATL1d6 protein (50-500 nM), El (ubiquitin activating enzyme), (10 nM), and E2 (20 kDa), (50-500 nM), and Mg ATP (2 mM) in 50 mM Tris-HCl, pH 8.0, for 2 minutes at 30° C. To assay for conjugate formation, the same reaction mixture is incubated at 37° C. and allowed to proceed for various times. Multi-ubiquitin conjugates and thiol ester linkages due to the activity of RATL1d6 are separated from free ubiquitin by polyacrylamide gel electrophoresis and visualized by autoradiography.

### Example 7

### Northern Analysis

Northern analysis is used to detect the presence of a transcript of a gene and involves the hybridization of a labeled nucleotide sequence to a membrane on which RNA from a particular cell or tissue type has been bound (See, J. Sambrook et al., supra). Analogous computer techniques using BLAST (S. F. Altschul, 1993, J. Mol. Evol., 36:290-300 and S. F. Altschul et al., 1990, J. Mol. Evol., 215:403-410) are used to search for identical or related molecules in nucleotide databases, such as GenBank or the LIFESEQ database (Incyte Pharmaceuticals). This analysis is much more rapid and less labor-intensive than performing multiple, membrane-based hybridizations. In addition, the sensitivity of the computer search can be modified to determine whether any particular match is categorized as being exact (identical) or homologous.

The basis of the search is the product score, which is defined as follows: (% sequence identity x maximum BLAST score)/100.

The product score takes into account both the degree of similarity between two sequences and the length of the sequence match. For example, with a product score of 40, the match will be exact within a 1-2% error; at 70, the match will be exact. Homologous molecules are usually identified by selecting those which show product scores between 15 and 40, although lower scores may identify related molecules. The results of Northern analysis are reported as a list of libraries in which the transcript encoding RATL1d6 occurs. Abundance and percent abundance are also reported. Abundance directly reflects the number of times that a particular transcript is represented in a cDNA library, and percent abundance is abundance divided by the total number of sequences that are examined in the cDNA library.

### Example 8

### Microarrays

For the production of oligonucleotides for a microarray, SEQ ID NO:1 is examined using a computer algorithm which starts at the 3′ end of the nucleotide sequence. The algorithm identifies oligomers of defined length that are unique to the gene, have a GC content within a range that is suitable for hybridization and lack predicted secondary structure that would interfere with hybridization. The algorithm identifies specific oligonucleotides of 20 nucleotides in length, i.e., 20-mers. A matched set of oligonucleotides is created in which one nucleotide in the center of each sequence is altered. This process is repeated for each gene in the microarray, and double sets of 20-mers are synthesized in the presence of fluorescent or radioactive nucleotides and arranged on the surface of a substrate. When the substrate is a silicon chip, a light-directed chemical process is used for deposition (WO 95/11995, M. Chee et al.).

Alternatively, a chemical coupling procedure and an ink jet device is used to synthesize oligomers on the surface of a substrate. (WO 95/25116, J. D. Baldeschweiler et al.). As another alternative, a “gridded” array that is analogous to a dot (or slot) blot is used to arrange and link cDNA fragments or oligonucleotides to the surface of a substrate using, for example, a vacuum system, or thermal, UV, mechanical, or chemical bonding techniques. A typical array may be produced by hand, or by using available materials and equipment, and may contain grids of 8 dots, 24 dots, 96 dots, 384 dots, 1536 dots, or 6144 dots. After hybridization, the microarray is washed to remove any non-hybridized probe, and a detection device is used to determine the levels and patterns of radioactivity or fluorescence. The detection device may be as simple as X-ray film, or as complicated as a light scanning apparatus. Scanned fluorescent images are examined to determine degree of complementarity and the relative abundance/expression level of each oligonucleotide sequence in the microarray.

### Example 9

### Production of Antibodies Specific for RATL1d6 Polypeptide

A RATL1d6 peptide conjugated to a carrier, such as BSA or KLH, or the full-length RATL1d6 protein, is used as an immunogen to raise antibodies in a host, such as rabbits or mice. As an alternative, a RATL1d6 fusion protein, i.e., RATL1d6 fused to GST or 6×HIS is expressed in an appropriate host expression system, such as bacteria, insect or mammalian cells and the resulting fusion product is isolated according to standard practice and used as an immunogen to generate polyclonal or monoclonal antibodies utilizing routine production methods and protocols known to those having skill in the art.

As another alternative, RATL1d6 polypeptide that is substantially purified using polyacrylamide gel electrophoresis (PAGE), (J. Sambrook, supra), or other purification techniques, is used to immunize rabbits and to produce antibodies using standard protocols. The amino acid sequence from SEQ ID NO:2 is analyzed using LASERGENE software (DNASTAR Inc.) to determine regions of high immunogenicity and one or more corresponding oligopeptides is synthesized and used to raise antibodies by means known and used by those having skill in the art. The selection of appropriate epitopes, such as those near the C-terminus, or in hydrophilic regions, is described by F. M. Ausubel et al., supra, as well as others.

Typically, the oligopeptides are 15 residues in length, synthesized using an ABI Peptide Synthesizer 431A (PE Biosystems) using fmoc-chemistry, and coupled to KLH (Sigma, St. Louis, Mo.) by reaction with N-maleimidobenzoyl-N-hydroxysuccinimide ester (MBS; F. M. Ausubel et al., supra). Rabbits are immunized with the oligopeptide-KLH complex in Freund's adjuvant. The resulting antisera are tested for anti-peptide activity, for example, by binding the peptide to plastic, blocking with 1% bovine serum albumin (BSA), reacting with the rabbit antisera, washing and reacting with radio-iodinated, or enzyme-labeled (e.g., horse radish peroxidase) goat or mouse anti-rabbit IgG immunoglobulin.

### Example 10

### Purification of Naturally Occurring RATL1d6 Polypeptide Using Specific Antibodies

Naturally occurring or recombinant RATL1d6 polypeptide is substantially purified by immunoaffinity chromatography using antibodies specific for RATLL d6 polypeptide. An immunoaffinity column is constructed by covalently coupling anti-RATL1d6 polypeptide antibody to an activated chromatographic resin, such as CNBr-activated SEPHAROSE (Amersham Pharmacia Biotech). After the coupling, the resin is blocked and washed according to the manufacturer's instructions.

Medium containing RATL1d6 polypeptide is passed over the immunoaffinity column, and the column is washed under conditions that allow the preferential absorbance of RATL1d6 polypeptide (e.g., high ionic strength buffers in the presence of detergent). The column is eluted under conditions that disrupt antibody/RATL1d6 polypeptide binding (e.g., a buffer of pH 2-3, or a high concentration of a chaotrope, such as urea or thiocyanate ion), and RATL1d6 polypeptide is collected.

### Example 11

### Identification of Molecules That Interact with the RATL1d6 Polypeptide

RATL1d6 polypeptide, or biologically active fragments thereof, are labeled with ¹²⁵I Bolton-Hunter reagent (Bolton et al., 1973, Biochem. J., 133:529). Candidate molecules previously arrayed in wells of a multi-welled plate are incubated with the labeled RATL1d6 polypeptide, washed, and any wells having labeled RATL1d6 polypeptide-candidate molecule complexes are assayed. Data obtained using different concentrations of RATL1d6 polypeptide are used to calculate values for the number, affinity and association of RATL1d6 polypeptide with the candidate molecules.

Another method suitable for identifying proteins, peptides or other molecules that interact with the RATL1d6 polypeptide include ligand binding assays such as the yeast-two hybrid system as described above.

### Example 12

### Creating N- and C-terminal Deletion Mutants Corresponding to the RATL1d6 Polypeptide of the Present Invention

As described elsewhere herein, the present invention encompasses the creation of N- and C-terminal deletion mutants, in addition to any combination of N- and C-terminal deletions thereof, corresponding to the RATL1d6 polypeptide of the present invention. A number of methods are available to one skilled in the art for creating such mutants. Such methods include a combination of PCR amplification and gene cloning methodology. Although one of skill in the art of molecular biology, through the use of the teachings provided or referenced herein, and/or otherwise known in the art as standard methods, could readily create each deletion mutants of the present invention, exemplary methods are described below.

Briefly, using the isolated cDNA clone encoding the full-length RATL1d6 polypeptide sequence, or splice variant sequences, appropriate primers of about 15-25 nucleotides derived from the desired 5′ and 3′ positions of SEQ ID NO:1 may be designed to PCR amplify, and subsequently clone, the intended N- and/or C-terminal deletion mutant. Such primers can comprise, for example, an initiation and stop codon for the 5′ and 3′ primer, respectively. Such primers can also comprise restriction sites to facilitate cloning of the deletion mutant post-amplification. Moreover, the primers can comprise additional sequences, such as, for example, flag-tag sequences, kozac sequences, or other sequences discussed and/or referenced herein.

For example, in the case of the D67 to G422 N-terminal deletion mutant, the following primers presented in Table 1 can be used to amplify a cDNA fragment corresponding to this deletion mutant:

TABLE 1

| | | |
|---|---|---|
| 5′ Primer | 5′-gcagca gcggccgc gacgagctgagctgcgagttcctgc-3′, | (SEQ ID NO:13) |
| | where the underlined sequence represents the NotI restriction | |
| | enzyme site. | |
| 3′ Primer | 5′-gcagca gtcgac gccgtcttcttttgggggtgtgtac-3′, | (SEQ ID NO:14) |
| | where the underlined sequence represents the SalI restriction | |
| | enzyme site. | |

In addition, in the case of the M1 to Q359 C-terminal deletion mutant, for example, the following primers presented in Table 2 can be used to amplify a cDNA fragment corresponding to this deletion mutant:

TABLE 2

| | | |
|---|---|---|
| 5′ Primer | 5′-gcagca gcggccgc atgcagcagccgcagccgcaggggc-3′, | (SEQ ID NO: 15) |
| | where the underlined sequence represents the NotI restriction | |
| | enzyme site. | |
| 3′ Primer | 5′-gcagca gtcgac gccctgtttggtgagaagttccatg-3′, | (SEQ ID NO:16) |
| | where the underlined sequence represents the SalI restriction | |
| | enzyme site. | |

Representative PCR amplification conditions are provided below, although the skilled artisan will appreciate that other conditions may be employed and/or required for efficient amplification. A 100 μl PCR reaction mixture can be prepared using 10 ng of the template DNA (cDNA clone of RATL1d6), 200 μM 4dNTPs, 1 μM primers, 0.25U Taq DNA polymerase (PE), and standard Taq DNA polymerase buffer. Typical PCR cycling condition are as follows:

| | |
|---|---|
| 20-25 cycles: | 45 seconds, 93 degrees |
| | 2 minutes, 50 degrees |
| | 2 minutes, 72 degrees |
| 1 cycle: | 10 minutes, 72 degrees |

After the final extension step of PCR, 5U Klenow Fragment can be added and incubated for 15 minutes at 30 degrees.

Upon digestion of the fragment with the NotI and SalI restriction enzymes, the fragment can be cloned into an appropriate expression and/or cloning vector which has been similarly digested (e.g., pSport1, among others). The skilled artisan will appreciate that other plasmids can be equally substituted, and may be desirable in certain circumstances. The digested fragment and vector are then ligated using a DNA ligase, and then used to transform competent E. coli cells, using methods provided herein and/or as otherwise known in the art.

The 5′ primer sequence for amplifying any additional N-terminal deletion mutants may be determined by reference to the following formula:

(S+(X*3)) to ((S+(X*3))+25), wherein ‘S’ is equal to the nucleotide position of the initiating start codon of the RATL1d6 gene (SEQ ID NO:1), and ‘X’ is equal to the most N-terminal amino acid of the intended N-terminal deletion mutant. The first term provides the start 5′ nucleotide position of the 5′ primer, while the second term provides the end 3′ nucleotide position of the 5′ primer corresponding to the sense strand of SEQ ID NO:1. Once the corresponding nucleotide positions of the primer are determined, the final nucleotide sequence can be created by the addition of applicable restriction site sequences to the 5′ end of the sequence, for example. As described herein, the addition of other sequences to the 5′ primer may be desired in certain circumstances (e.g., kozac sequences, etc.).

The 3′ primer sequence for amplifying any additional N-terminal deletion mutants can be determined by reference to the following formula:

(S+(X*3)) to ((S+(X*3))−25), wherein ‘S’ is equal to the nucleotide position of the initiating start codon of the RATL1d6 gene (SEQ ID NO:1), and ‘X’ is equal to the most C-terminal amino acid of the intended N-terminal deletion mutant. The first term provides the start 5′ nucleotide position of the 3′ primer, while the second term provides the end 3′ nucleotide position of the 3′ primer corresponding to the antisense strand of SEQ ID NO:1. Once the corresponding nucleotide positions of the primer are determined, the final nucleotide sequence can be created by the addition of applicable restriction site sequences to the 5′ end of the sequence, for example. As described herein, the addition of other sequences to the 3′ primer may be desired in certain circumstances (e.g., stop codon sequences, etc.). The skilled artisan will appreciate that modifications to the above nucleotide positions may be necessary for optimizing PCR amplification.

The same general formulas provided above can be used in identifying the 5′ and 3′ primer sequences for amplifying any C-terminal deletion mutant of the present invention. Moreover, the same general formulas provided above may be used in identifying the 5′ and 3′ primer sequences for amplifying any combination of N-terminal and C-terminal deletion mutant of the present invention. The skilled artisan will appreciate that modifications of the above nucleotide positions may be necessary for optimizing PCR amplification.

Preferably, the following N-terminal RATL1d6 deletion polypeptides of SEQ ID NO:2 are encompassed by the present invention: M1-G422, Q2-G422, Q3-G422, P4-G422, Q5-G422, P6-G422, Q7-G422, G8-G422, Q9-G422, Q10-G422, Q11-G422, P12-G422, G13-G422, P14-G422, G15-G422, Q16-G422, Q17-G422, L18-G422, G19-G422, G20-G422, Q21-G422, G22-G422, A23-G422, A24-G422, P25-G422, G26-G422, A27-G422, G28-G422, G29-G422, G30-G422, P31-G422, G32-G422, G33-G422, G34-G422, P35-G422, G36-G422, P37-G422, G38-G422, P39-G422, C40-G422, L41-G422, R42-G422, R43-G422, E44-G422, L45-G422, K46-G422, L47-G422, L48-G422, E49-G422, S50-G422, I51-G422, F52-G422, H53-G422, R54-G422, G55-G422, H56-G422, E57-G422, R58-G422, F59-G422, R60-G422, I61-G422, A62-G422, S63-G422, A64-G422, C65-G422, L66-G422, D67-G422, E68-G422, L69-G422, S70-G422, C71-G422, E72-G422, F73-G422, L74-G422, L75-G422, A76-G422, G77-G422, A78-G422, G79-G422, G80-G422, A81-G422, G82-G422, A83-G422, G84-G422, A85-G422, A86-G422, P87-G422, G88-G422, P89-G422, H90-G422, L91-G422, P92-G422, P93-G422, R94-G422, G95-G422, S96-G422, V97-G422, P98-G422, G99-G422, D100-G422, P101-G422, V102-G422, R103-G422, I104-G422, H105-G422, C106-G422, N107-G422, I108-G422, T109-G422, E10-G422, S111-G422, Y112-G422, P113-G422, A114-G422, V115-G422, P116-G422, P117-G422, I118-G422, W119-G422, S120-G422, V121-G422, E122-G422, S123-G422, D124-G422, D125-G422, P126-G422, N127-G422, L128-G422, A129-G422, A130-G422, V131-G422, L132-G422, E133-G422, R134-G422, L135-G422, V136-G422, D137-G422, I138-G422, K139-G422, K140-G422, G141-G422, N142-G422, T143-G422, L144-G422, L145-G422, L146-G422, Q147-G422, H148-G422, L149-G422, K150-G422, R151-G422, I152-G422, I153-G422, S154-G422, D155-G422, L156-G422, C157-G422, K158-G422, L159-G422, Y160-G422, N161-G422, L162-G422, P163-G422, Q164-G422, H165-G422, P166-G422, D167-G422, V168-G422, E169-G422, M170-G422, L171-G422, D172-G422, Q173-G422, P174-G422, L175-G422, P176-G422, A177-G422, E178-G422, Q179-G422, C180-G422, T181-G422, Q182-G422, E183-G422, D184-G422, V185-G422, S186-G422, S187-G422, E188-G422, D189-G422, E190-G422, D191-G422, E192-G422, E193-G422, M194-G422, P195-G422, E196-G422, D197-G422, T198-G422, E199-G422, D200-G422, L201-G422, D202-G422, H203-G422, Y204-G422, E205-G422, M206-G422, K207-G422, E208-G422, E209-G422, E210-G422, P211-G422, A212-G422, E213-G422, G214-G422, K215-G422, K216-G422, S217-G422, E218-G422, D219-G422, D220-G422, G221-G422, I222-G422, G223-G422, K224-G422, E225-G422, N226-G422, L227-G422, A228-G422, I229-G422, L230-G422, E231-G422, K232-G422, I233-G422, K234-G422, K235-G422, N236-G422, Q237-G422, R238-G422, Q239-G422, D240-G422, Y241-G422, L242-G422, N243-G422, G244-G422, A245-G422, V246-G422, S247-G422, G248-G422, S249-G422, V250-G422, Q251-G422, A252-G422, T253-G422, D254-G422, R255-G422, L256-G422, M257-G422, K258-G422, E259-G422, L260-G422, R261-G422, D262-G422, I263-G422, Y264-G422, R265-G422, S266-G422, Q267-G422, S268-G422, F269-G422, K270-G422, G271-G422, G272-G422, N273-G422, Y274-G422, A275-G422, V276-G422, E277-G422, L278-G422, V279-G422, N280-G422, D281-G422, S282-G422, L283-G422, Y284-G422, D285-G422, W286-G422, N287-G422, V288-G422, K289-G422, L290-G422, L291-G422, K292-G422, V293-G422, D294-G422, Q295-G422, D296-G422, S297-G422, A298-G422, L299-G422, H300-G422, N301-G422, D302-G422, L303-G422, Q304-G422, 1305-G422, L306-G422, K307-G422, E308-G422, K309-G422, E310-G422, G311-G422, A312-G422, D313-G422, F314-G422, I315-G422, L316-G422, L317-G422, N318-G422, F319-G422, S320-G422, F321-G422, K322-G422, D323-G422, N324-G422, F325-G422, P326-G422, F327-G422, D328-G422, P329-G422, P330-G422, F331-G422, V332-G422, R333-G422, V334-G422, V335-G422, S336-G422, P337-G422, V338-G422, L339-G422, S340-G422, G341-G422, G342-G422, Y343-G422, V344-G422, L345-G422, G346-G422, G347-G422, G348-G422, A349-G422, 1350-G422, C351-G422, M352-G422, E353-G422, L354-G422, L355-G422, T356-G422, K357-G422, Q358-G422, G359-G422, W360-G422, S361-G422, S362-G422, A363-G422, Y364-G422, S365-G422, 1366-G422, E367-G422, S368-G422, V369-G422, 1370-G422, M371-G422, Q372-G422, 1373-G422, S374-G422, A375-G422, T376-G422, L377-G422, V378-G422, K379-G422, G380-G422, K381-G422, A382-G422, R383-G422, V384-G422, Q385-G422, F386-G422, G387-G422, A388-G422, N389-G422, K390-G422, S391-G422, Q392-G422, Y393-G422, S394-G422, L395-G422, T396-G422, R397-G422, A398-G422, Q399-G422, Q400-G422, S401-G422, Y402-G422, K403-G422, S404-G422, L405-G422, V406-G422, Q407-G422, 1408-G422, H409-G422, E41 O-G422, K411-G422, N412-G422, G413-G422, W414-G422, Y415-G422, and/or T416-G422 (of SEQ ID NO:2). Polynucleotide sequences encoding these polypeptides are also provided. One or more of these N-terminal RATL1d6 deletion polypeptides can be employed as immunogenic and/or antigenic epitopes as described elsewhere herein.

Also, preferably, the following C-terminal RATL1d6 deletion polypeptides of SEQ ID NO:2 are encompassed by the present invention: M1-G422, M1-D421, M1-E420, M1-K419, M1-P418, M1-P417, M1-T416, M1-Y415, M1-W414, M1-G413, M1-N412, M1-K411, M1-E410, M1-H409, M1-1408, M1-Q407, M1-V406, M1-L405, M1-S404, M1-K403, M1-Y402, M1-S401, M1-Q400, M1-Q399, M1-A398, M1-R397, M1-T396, M1-L395, M1-S394, M1-Y393, M1-Q392, M1-S391, M1-K390, M1-N389, M1-A388, M1-G387, M1-F386, M1-Q385, M1-V384, M1-R383, M1-A382, M1-K381, M1-G380, M1-K379, M1-V378, M1-L377, M1-T376, M1-A375, M1-S374, M1-I373, M1-Q372, M1-M371, M1-1370, M1-V369, M1-S368, M1-E367, M1-I366, M1-S365, M1-Y364, M1-A363, M1-S362, M1-S361, M1-W360, M1-G359, M1-Q358, M1-K357, M1-T356, M1-L355, M1-L354, M1-E353, M1-M352, M1-C351, M1-1350, M1-A349, M1-G348, M1-G347, M1-G346, M1-L345, M1-V344, M1-Y343, M1-G342, M1-G341, M1-S340, M1-L339, M1-V338, M1-P337, M1-S336, M1-V335, M1-V334, M1-R333, M1-V332, M1-F331, M1-P330, M1-P329, M1-D328, M1-F327, M1-P326, M1-F325, M1N324, M1-D323, M1-K322, M1-F321, M1-S320, M1-F319, M1-N318, M1-L317, M1-L316, M1-1315, M1-F314, M1-D313, M1-A312, M1-G311, M1-E310, M1-K309, M1-E308, M1-K307, M1-L306, M1-1305, M1-Q304, M1-L303, M1-D302, M1-N301, M1-H300, M1-L299, M1-A298, M1-S297, M1 D296, M1-Q295, M1-D294, M1-V293, M1-K292, M1-L291, M1-L290, M1-K289, M1-V288, M1-N287, M1-W286, M1-D285, M1-Y284, M1-L283, M1-S282, M1-D281, M1-N280, M1-V279, M1-L278, M1-E277, M1-V276, M1-275, M1-Y274, M1-N273, M1-G272, M1-G271, M1-K270, M1-F269, M1-S268, M1-Q267, M1-S266, M1-R265, M1-Y264, M1-1263, M1-D262, M1-R261, M1-L260, M1-E259, M1-K258, M1-M257, M1-L256, M1-R255, M1-D254, M1-T253, M1-A252, M1-Q251, M1-V250, M1-S249, M1-G248, M1-S247, M1-V246, M1-A245, M1-G244, M1-N243, M1-L242, M1-Y241, M1-D240, M1-Q239, M1-R238, M1-Q237, M1-N236, M1-K235, M1-K234, M1-I233, M1-K232, M1-E231, M1-L230, M1-I229, M1-A228, M1-L227, M1-N226, M1-E225, M1-K224, M1-G223, M1-1222, M1-G221, M1-D220, M1-D219, M1-E218, M1-S217, M1-K216, M1-K215, M1-G214, M1-E213, M1-A212, M1-P211, M1-E210, M1-E209, M1-E208, M1-K207, M1-M206, M1-E205, M1-Y204, M1-H203, M1-D202, M1-L201, M1-D200, M1-E199, M1-T198, M1-D197, M1-E196, M1-P195, M1-M194, M1-E193, M1-E192, M1-D191, M1-E190, M1-D189, M1-E188, M1-S187, M1-S186, M1-V185, M1-D184, M1-E183, M1-Q182, M1-T181, M1-C18O, M1-Q179, M1-E178, M1-A177, M1-P176, M1-L175, M1-P174, M1-Q173, M1-D172, M1-L171, M1-M170, M1-E169, M1-V168, M1-D167, M1-P166, M1-H165, M1-Q164, M1-P163, M1-L162, M1-N161, M1-Y160, M1-L159, M1-K158, M1-C157, M1-L156, M1-D155, M1-S154, M1-1153, M1-1152, M1-R151, M1-K150, M1-L149, M1-H148, M1-Q147, M1-L146, M1-L145, M1-L144, M1-T143, M1-N142, M1-G141, M1-K140, M1-K139, M1-1138, M1-D137, M1-V136, M1-L135, M1-R134, M1-E133, M1-L132, M1-V131, M1-A130, M1-A129, M1-L128, M1-N127, M1-P126, M1-D125, M1-D124, M1-S123, M1-E122, M1V121, M1-S120, M1-W119, M1-I118, M1-P117, M1-P116, M1-V115, M1A114, M1-P113, M1-Y112, M1-S111, M1-E110, M1-T109, M1-1108, M1-N₁₀₇, M1-C106, M1-H105, M1-I104, M1-R103, M1-V102, M1-P101, M1-D100, M1-G99, M1-P98, M1-V97, M1-S96, M1-G95, M1-R94, M1-P93, M1-P92, M1-L91, M1-H90, M1-P89, M1-G88, M1-P87, M1-A86, M1-A85, M1-G84, M1-A83, M1-G82, M1-A81, M1-G80, M1-G79, M1-A78, M1-G77, Ml A76, M1-L75, M1-L74, M1-F73, M1-E72, M1-C71, M1-S70, M1-L69, M1-E68, M1-D67, M1-L66, M1-C65, M1-A64, M1-S63, M1-A62, M1-161, M1-R60, M1-F59, M1-R58, M1-E57, M1-H56, M1-G55, M1-R54, M1-H53, M1-F52, M1-151, M1-S50, M1-E49, M1-L48, M1-L47, M1-K46, M1-L45, M1-E44, M1-R43, M1-R42, M1-L41, M1-C40, M1-P39, M1-G38, M1-P37, M1-G36, M1-P35, M1-G34, M1-G33, M1-G32, M1-P31, M1-G30, M1-G29, M1-G28, M1-A27, M1-G26, M1-P25, M1-A24, M1-A23, M1-G22, M1-Q21, M1-G20, M1-G19, M1-L18, M1-Q17, M1-Q16, M1-G15, M1-P14, M1-G13, M1-P12, M1-Q11, M1-Q10, M1-Q9, M1-G8, and/or M1-Q7 (of SEQ ID NO:2). Polynucleotide sequences encoding these polypeptides are also provided. One or more of these C-terminal RATL1d6 deletion polypeptides can be used as immunogenic and/or antigenic epitopes as described elsewhere herein.

Alternatively, preferred polypeptides/peptides of the present invention comprise polypeptide/peptide sequences corresponding to, for example, internal regions of the RATL1d6 polypeptide (e.g., any combination of both N- and C-terminal RATL1d6 polypeptide deletions) of SEQ ID NO:2. For example, internal regions can be defined by the equation: amino acid “NX” to amino acid “CX”, where “NX” refers to any N-terminal deletion polypeptide amino acid of RATL1d6 (SEQ ID NO:2), and where “CX” refers to any C-terminal deletion polypeptide amino acid of RATL1d6 (SEQ ID NO:2). Polynucleotides encoding these polypeptides are also provided. The present invention also encompasses the use of these polypeptides as an immunogenic and/or antigenic epitope as described elsewhere herein.

### Example 13

### Expression Profiling of the Human RATL1D6 Polypeptide

The following PCR primer pair was used to measure the steady state levels of RATL1 d6 mRNA by quantitative PCR:

| | |
|---|---|
| Sense: | |
| 5′-aggatcatctccgacctgtg-3′ | (SEQ ID NO:48) |
| Antisense: | |
| 5′-caagggttgatccagcatct-3′ | (SEQ ID NO:49) |

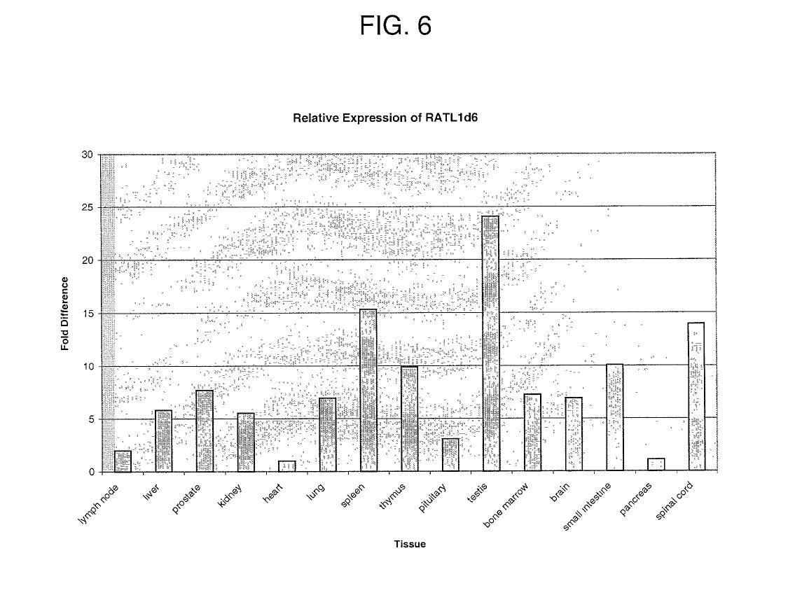

Briefly, first strand cDNA was produced from commercially available mRNA (Clontech, Palo Alto, Calif.), i.e., the mRNA obtained from the 15 tissues analyzed in the expression profile of FIG. 6. The relative amount of cDNA used in each assay was determined by performing a parallel experiment using a primer pair for a gene expressed in equal amounts in all tissues, i.e., cyclophilin. The cyclophilin primer pair detected small variations in the amount of cDNA in each sample and these data were used for normalization of the data obtained with the primer pair for the RATL1d6 gene.

The PCR data were converted into a relative assessment of the differences in transcript abundance among the tissues tested, as presented in FIG. 6. Transcripts corresponding to the ubiquitin conjugating enzyme, RATL1d6, were expressed at high levels in many tissues: predominately in testis, spleen, and spinal cord; significantly in thymus, small intestine, and to a lesser extent in prostate, lung, bone marrow, kidney, brain, liver, heart, lymph node, pituitary, and pancreas tissues. The ubiquitous expression of the RATL1d6 polypeptide is consistent with its important role as a ubiquitin conjugating enzyme, and affirms its function as an essential enzyme in a variety of cellular processes.

### Example 14

### Functional Studies of RATL1d6 Involving Drosophila Ortholog EG:25E8.2 In An LPS-Inducible Luciferase Reporter System

A stable S2 cell line was generated with an LPS-responsive AttacinD promoter fused to a luciferase reporter (modified as described below from Tauszig, S. et al., 2000, Proc. Natl. Acad. Sci. USA, 97:10520-10525). S2 cells were purchased from Invitrogen and maintained at 25° C. in complete 1×Schneider's Drosophila medium (Cat. No. 11720-034, Invitrogen, former GIBCO BRL) supplemented to contain 10% heat-inactivated fetal bovine serum (Cat. No. 10100-147, Invitrogen, former GIBCO BRL), 100 units/ml of penicillin, 100 μg/ml of streptomycin (100×stock of Penicillin-Streptomycin, Cat. No. 15140-148, from Invitrogen, former GIBCO BRL) and 20 mM L-Glutamine (100×L-Glutamine, Cat. No. 25030-149, from Invitrogen, former GIBCO BRL).

A 1.6 Kb promoter region of the attacinD AMP gene was isolated from S2 genomic DNA by PCR using the primer pair: 5′-atgaggcttggatcagcttt-3′ (SEQ ID NO:50), (forward, 157904-157923 bp of AE003718 Drosophila Genome project) and 5′-cctgaagcctgacattccat-3′ (SEQ ID NO:51), (reversed, 159547-159566 bp of AE003718). Primers were obtained from GIBCOBRL. PCR conditions were as follows: 96° C., 4 min; 94° C., 2 min; 55° C., 45 seconds; 72° C., 2 min; PCR 35 cycles. The 1.6 kb attacinD PCR fragment was subcloned into a pCR2.1-TOPO vector (TOPO TA Cloning Kits, Cat. No. K4500-01, Invitrogen). The attacinD promoter was subcloned from pCR2.1-TOPO vector into the pGL3-Enhancer luciferase vector with restriction enzymes SacI and XhoI (pGL3-Enhancer luciferase reporter vector, Cat. No. E1771, Promega). A similar region was shown to be LPS responsive in a reporter assay (Tauszig, S. et al., 2000, Proc. Natl. Acad. Sci. USA, 97:10520-10525).

A final transfection construct, pGL3-enhancer-attacinD, was co-transfected using known calcium phosphate method with pCoHYGRO plasmid providing the hygromycin-B resistant gene as a stable selection. This construct was used to transfect S2 cells (Inducible DES Kit, Cat. No. K4120-01, Drosophila Expression System Instruction Manual, Invitrogen).

Briefly 19 μg of pGL3-enhancer-attacinD DNA was mixed with 1 μg of pCoHYGRO DNA and transfection buffer and the mixture was used to transfect 6-12×10⁶ cells/3 mls/well in a 6-well Falcon tissue culture plate. Stable cells were selected and maintained in complete Schneider's medium containing 300 μg/ml Hygromycin-B (Cat. No. R220-05, Invitrogen). Stable lines were tested for responsiveness to LPS (Han, Z. S. and Ip, Y. T., 1999, J. Biol. Chem., 274:21355-21361). Cells were treated with 20 μg/ml LPS (Cat. No. L-2654, Sigma) for 5 hours. Luciferase expression was assayed with Bright-Glo™ Luciferase Assay System (Cat. No. E2620, Promega) and the luminescence signal was detected by a 1450 MICROBETA Wallac Jet Liquid Scintillation & Luminescence Counter (Perkin Elmer Life Sciences). Two stable AttD-luc reporter cell lines (E4-1 and E4-9) were obtained after three rounds of limiting dilution and used for further studies.

RNAi constructs were made for EG:25E8.2 and control genes as follows: Complementary DNA (cDNA) clones for Drosophila genes were obtained from Research Genetics, Inc (St. Louis, Mo.). These included the cDNAs from Relish (EST GH01881), IKKB (EST LD21354), Cactus (LD18620), and EG:25E8.2 (LD09991) (Rubin, G. M. et al., 2000, Science, 287:2222-2224). Double-stranded RNAi was generated following a modified protocol of (Hammond, S. M. et al., 2000, Nature, 404:293-0296). Briefly, dsRNA was synthesized from a template amplified by PCR with T7 promoter sequences flanking the cDNA insert using the MEGAscript™ T7 High Yield Transcription Kit (Cat. No. 1334, Ambion). GH0881 and LD 21354 were used in the pOT2 vector,

(forward primer: 5′-actgcagccgattcattaatg-3′, (SEQ ID NO:52), (reverse primer:

5′-gaattaatacgactcactatagggagatatcatacacatacgatttag-3′), (SEQ ID NO:53); and LD18620 and CG 2924 were used in a pBS vector (forward primer: 5′-gaattaatacgactcactatagggagacatgattacgccaagctcgaa-3′), (SEQ ID NO:54);

(reverse primer: 5′-tgtaaaacgacggccagtgaa-3′), (SEQ ID NO:55). Double stranded RNA (dsRNA) was diluted at 1:5 and denatured prior to addition to E4-1 and E4-9 cells.

Transfection of dsRNA into S2 cells was performed by adding dsRNA directly into S2 cells in serum free medium (Clemens, J. C. et al., 2000, Proc. Nat. Acad. Sci. USA, 97:6499-6503). Prior to transfection, the cells were passaged about 24 hours before transfection at 1×10⁶ cells/ml in complete 1×Schneider medium. Immediately preceding the transfection, the cells were washed twice with serum free DES Expression Medium (Cat. No. Q500-01, Invitrogen) and resuspended in serum-free DES medium at 7×10⁵ cells/ml.

100 μl of cells were added to each well in 96-well tissue culture plates (Falcon). Thereafter, 5 μl of dsRNA/well were added, followed by vigorous shaking for 45 minutes to 1 hour. Finally, 150 μl complete 1×Schneider medium/well was added. The 96-well plates were covered with Saran Wrap before incubating at 25° C. After 3 days of incubation, each of the dsRNA treated cells were split into duplicates for the luciferase assay, and into triplicates for the proliferation assay.

5-15 μl of cells in 100 μl total volume for were used for the luciferase assay, and 30-35 μl of cells in 100 μl total volume were used for the proliferation assay. Luciferase assay plates were incubated for 5 hours after adding LPS at 20 μg/ml. Proliferation assay plates were incubated for 2-3 hours before reading at 490 nm Optical Density. (CellTiter 96 Aqueous One Solution Cell Proliferation Assay from Promega, Cat. No. G3580).

The results presented in Table 3 represent the results of one experiment with E4-1 cells averaged in duplicate relative to control samples. Changes are relative to 1.00, with 1.00 representing the Control sample's relative luciferase activity after normalization with cell number obtained in the proliferation assay, as averaged from several experiments. Similar results were obtained in multiple, repeat experiments and with the E4-9 stable cell line. In Table 3, NS signifies non-stimulated and LPS denotes the LPS treatment as described above.

TABLE 3

| | NS | LPS |
|---|---|---|
| Control | 1.2 | 0.8 |
| EG:25E8.2 | 4.85 | 6.9 |
| (Drosophila | | |
| ortholog) | | |
| IKK-B | 0.33 | 0.2 |
| Relish | 0.12 | 0.01 |
| Cactus | 6.9 | 9.65 |

The results from these analyses as presented in Table 3 indicate that EG:25E8.2 serves as a negative regulator in an innate immunity model in Drosophila cells, with a similar profile as cactus (IKB). RNAi experiments of positive regulators of this signaling pathway were found to have low luciferase activity as demonstrated with IKKB and Relish (Silverman, N. et al., 2000, Genes Dev., 14:2461-2471). These results demonstrate that EG:25E8.2 does regulate the LPS-response pathway. Similarly, RATL1d6, due to its similarity to EG:25E8.2, is expected to have a similar role in mammalian immunity pathways.

### Example 15

### Site Directed/Site-Specific Mutagenesis

In vitro site-directed mutagenesis is an invaluable technique for studying protein structure-function relationships and gene expression, for example, as well as for vector modification. Approaches utilizing single stranded DNA (ssDNA) as the template have been reported (e.g., T. A. Kunkel et al., 1985, Proc. Natl. Acad. Sci. USA), 82:488-492; M. A. Vandeyar et al., 1988, Gene, 65(1):129-133; M. Sugimoto et al., 1989, Anal. Biochem., 179(2):309-311; and J. W. Taylor et al., 1985, Nuc. Acids. Res., 13(24):8765-8785).

The use of PCR in site-directed mutagenesis accomplishes strand separation by using a denaturing step to separate the complementary strands and to allow efficient polymerization of the PCR primers. PCR site-directed mutagenesis methods thus permit site specific mutations to be incorporated in virtually any double stranded plasmid, thus eliminating the need for re-subcloning into M13-based bacteriophage vectors or single-stranded rescue. (M. P. Weiner et al., 1995, Molecular Biology: Current Innovations and Future Trends, Eds. A.M. Griffin and H.G. Griffin, Horizon Scientific Press, Norfolk, UK; and C. Papworth et al., 1996, Strategies, 9(3):3-4).

A protocol for performing site-directed mutagenesis, particularly employing the QuikChange™ site-directed mutagenesis kit (Stratagene, La Jolla, Calif.; U.S. Pat. Nos. 5,789,166 and 5,923,419) is provided for making point mutations, to switch or substitute amino acids, and to delete or insert single or multiple amino acids, particularly in the RATL1d6 amino acid sequence of this invention.

Primer Design

For primer design using this protocol, the mutagenic oligonucleotide primers are designed individually according to the desired mutation. The following considerations should be made for designing mutagenic primers: 1) Both of the mutagenic primers must contain the desired mutation and anneal to the same sequence on opposite strands of the plasmid; 2) Primers should be between 25 and 45 bases in length, and the melting temperature (Tₘ) of the primers should be greater than, or equal to, 78° C. The following formula is commonly used for estimating the Tₘ of primers: T=81.5+0.41 (% GC)−675/N−% mismatch. For calculating Tₘ, N is the primer length in bases; and values for % GC and % mismatch are whole numbers. For calculating Tₘ for primers intended to introduce insertions or deletions, a modified version of the above formula is employed: T=81.5+0.41 (% GC)−675/N, where N does not include the bases which are being inserted or deleted; 3) The desired mutation (deletion or insertion) should be in the middle of the primer with approximately 10-15 bases of correct sequence on both sides; 4) The primers optimally should have a minimum GC content of 40%, and should terminate in one or more C or G bases; 5) Primers need not be 5′-phosphorylated, but must be purified either by fast polynucleotide liquid chromatography (FPLC) or by polyacrylamide gel electrophoresis (PAGE). Failure to purify the primers results in a significant decrease in mutation efficiency; and 6) It is important that primer concentration is in excess. It is suggested that the amount of template be varied, while the concentration of the primers is kept constantly in excess (QuikChange™ Site-Directed Mutagenesis Kit, Stratagene, La Jolla, Calif.).

Protocol for Setting Up the Reactions

Using the above-described primer design, two complimentary oligonucleotides containing the desired mutation, flanked by unmodified nucleic acid sequence, are synthesized. The resulting oligonucleotide primers are purified.

A control reaction is prepared using 5 μl 10×reaction buffer (100 mM KCl; 100 mM (NH₄)₂SO₄; 200 mM Tris-HCl, pH 8.8; 20 mM MgSO₄; 1% Triton® X-100; 1 mg/ml nuclease-free bovine serum albumin, BSA); 2 μl (10 ng) of pWhitescript™, 4.5-kb control plasmid (5 ng/μl); 1.25 μl (125 ng) of oligonucleotide control primer #1 (34-mer, 100 ng/μl); 1.25 μl (125 ng) of oligonucleotide control primer #2 (34-mer, 100 ng/μl); 1 μl of dNTP mix; double distilled H₂O; to a final volume of 50 μl. Thereafter, 1 μl of DNA polymerase (PfuTurbo® DNA Polymerase, Stratagene), (2.5U/μl) is added. PfuTurbo® DNA Polymerase is stated to have 6-fold higher fidelity in DNA synthesis than does Taq polymerase. To maximize temperature cycling performance, use of thin-walled test tubes is suggested to ensure optimum contact with the heating blocks of the temperature cycler.

The sample reaction is prepared by combining 5 μl of 10×reaction buffer; x μl (5-50 ng) of dsDNA template; ×μl (125 ng) of oligonucleotide primer #1; x μl (5-50 ng) of dsDNA template; x μl (125 ng) of oligonucleotide primer #2; 1 μl of dNTP mix; and ddH₂O to a final volume of 50 μl. Thereafter, 1 μl of DNA polymerase (PfuTurbo DNA Polymerase, Stratagene), (2.5U/μl) is added.

It is suggested that if the thermal cycler does not have a hot-top assembly, each reaction should be overlaid with approximately 30 μl of mineral oil.

Cycling the Reactions

Each reaction is cycled using the following cycling parameters:

| Segment | Cycles | Temperature | Time |
|---|---|---|---|
| 1 | 1 | 95° C. | 30 seconds |
| 2 | 12-18 | 95° C. | 30 seconds |
| | | 55° C. | 1 minute |
| | | 68° C. | 2 minutes/kb of |
| | | | plasmid length |

For the control reaction, a 12-minute extension time is used and the reaction is run for 12 cycles. Segment 2 of the above cycling parameters is adjusted in accordance with the type of mutation desired. For example, for point mutations, 12 cycles are used; for single amino acid changes, 16 cycles are used; and for multiple amino acid deletions or insertions, 18 cycles are used. Following the temperature cycling, the reaction is placed on ice for 2 minutes to cool the reaction to ≦37° C. Digesting the Products and Transforming Competent Cells

One μl of the DpnI restriction enzyme (10U/μl) is added directly (below mineral oil overlay) to each amplification reaction using a small, pointed pipette tip. The reaction mixture is gently and thoroughly mixed by pipetting the solution up and down several times. The reaction mixture is then centrifuged for 1 minute in a microcentrifuge. Immediately thereafter, each reaction is incubated at 37° C. for 1 hour to digest the parental (i.e., the non-mutated) supercoiled dsDNA.

Competent cells (i.e., XL1-Blue supercompetent cells, Stratagene) are thawed gently on ice. For each control and sample reaction to be transformed, 50 μl of the supercompetent cells are aliquotted to a prechilled test tube (Falcon 2059 polypropylene). Next, 1 μl of the DpnI-digested DNA is transferred from the control and the sample reactions to separate aliquots of the supercompetent cells. The transformation reactions are gently swirled to mix and incubated for 30 minutes on ice. Thereafter, the transformation reactions are heat-pulsed for 45 seconds at 42° C. for 2 minutes.

0.5 ml of NZY+ broth, preheated to 42° C. is added to the transformation reactions which are then incubated at 37° C. for 1 hour with shaking at 225-250 rpm. An aliquot of each transformation reaction is plated on agar plates containing the appropriate antibiotic for the vector. For the mutagenesis and transformation controls, cells are spread on LB-ampicillin agar plates containing 80 μg/ml of X-gal and 20 mM MIPTG. Transformation plates are incubated for >16 hours at 37° C.

TABLE 4
Sequence Listing Description

| SEQ ID NO: | Description |
|---|---|
| SEQ ID NO: 1 | RATL1d6 nucleic acid sequence |
| | (FIG. 1) |
| SEQ ID NO: 2 | RATL1d6 polypeptide sequence |
| | (FIG. 3) |
| SEQ ID NO: 3 | C. elegans ortholog F2H2.8 |
| | (FIG. 4) |
| SEQ ID NO: 4 | Drosophila ortholog EG: 25E8.2 |
| | (FIG. 4) |
| SEQ ID NO: 5 | E2 UBC P52483/mouse UB6B |
| | (FIG. 4) |
| SEQ ID NO: 6 | P27924/human UBC1/Huntingtin |
| | interacting protein (HIP) (FIG. 4) |
| SEQ ID NO: 7 | CAA72184/Drosophila UBCD4 |
| | (FIG. 4) |
| SEQ ID NO: 8 | P14682/yeast UBC3/CDC34 |
| | (FIG. 4) |
| SEQ ID NO: 9 | PCR primer PY508, |
| | 5′-tgcagtgtctggctcggtgc-3′ |
| SEQ ID NO: 10 | PCR primer PY509, |
| | 5′-ctgatctgcatgatcactgac-3′ |
| SEQ ID NO: 11 | oligonucleotide PY495 |
| | 5′- |
| | tccactgcaacatcacggagtcataccctg- |
| | 3′ |
| SEQ ID NO: 12 | oligonucleotide PY496 |
| | 5′- |
| | atgcagtcgaactcgtgaatgacagtctgt-3′ |
| SEQ ID NO: 13 | 5′ primer, N-terminal deletion |
| | (Example 12) |
| SEQ ID NO: 14 | 3′ primer, N-terminal deletion |
| | (Example 12) |
| SEQ ID NO: 15 | 5′ primer, C-terminal deletion |
| | (Example 12) |
| SEQ ID NO: 16 | 3′ primer, C-terminal deletion |
| | (Example 12) |
| SEQ ID NO: 17 | RATL1d6 polypeptide |
| | transmembrane domain |
| SEQ ID NO: 18 | PKC phosphorylation site |
| | polypeptide, |
| | GSVQATDRLMKEL |
| SEQ ID NO: 19 | PKC phosphorylation site |
| | polypeptide, |
| | IYRSQSFKGGNYA |
| SEQ ID NO: 20 | PKC phosphorylation site |
| | polypeptide, |
| | ILLNFSFKDNFPF |
| SEQ ID NO: 21 | PKC phosphorylation site |
| | polypeptide, |
| | TRAQQSYKSLVQI |
| SEQ ID NO: 22 | Casein kinase II phosphorylation |
| | site polypeptide, |
| | PAEQCTQEDVSSED |
| SEQ ID NO: 23 | Casein kinase II phosphorylation |
| | site polypeptide, |
| | TQEDVSSEDEDEEM |
| SEQ ID NO: 24 | Casein kinase II phosphorylation |
| | site polypeptide, |
| | QEDVSSEDEDEEMP |
| SEQ ID NO: 25 | Casein kinase II phosphorylation |
| | site polypeptide, |
| | AEGKKSEDDGIGKE |
| SEQ ID NO: 26 | Casein kinase II phosphorylation |
| | site polypeptide, |
| | ELVNDSLYDWNVKL |
| SEQ ID NO: 27 | Casein kinase II phosphorylation |
| | site polypeptide, |
| | ILLNFSFKDNFPFD |
| SEQ ID NO: 28 | Asparagine glycosylation site |
| | polypeptide, |
| | VRIHCNITESYPAV |
| SEQ ID NO: 29 | Asparagine glycosylation site |
| | polypeptide, |
| | AVELVNDSLYDWNV |
| SEQ ID NO: 30 | Asparagine glycosylation site |
| | polypeptide, |
| | DFILLNFSFKDNFP |
| SEQ ID NO: 31 | Asparagine glycosylation site |
| | polypeptide, |
| | VQFGANKSQYSLTR |
| SEQ ID NO: 32 | N-myristylation site polypeptide, |
| | QQPGPGQQLGGQGAAP |
| SEQ ID NO: 33 | N-myristylation site polypeptide, |
| | PGQQLGGQGAAPGAGG |
| SEQ ID NO: 34 | N-myristylation site polypeptide, |
| | QLGGQGAAPGAGGGPG |
| SEQ ID NO: 35 | N-myristylation site polypeptide, |
| | AAPGAGGGPGGGPGPG |
| SEQ ID NO: 36 | N-myristylation site polypeptide, |
| | APGAGGGPGGGPGPGP |
| SEQ ID NO: 37 | N-myristylation site polypeptide, |
| | EFLLAGAGGAGAGAAP |
| SEQ ID NO: 38 | N-myristylation site polypeptide, |
| | LLAGAGGAGAGAAPGP |
| SEQ ID NO: 39 | N-myristylation site polypeptide, |
| | LAGAGGAGAGAAPGPH |
| SEQ ID NO: 40 | N-myristylation site polypeptide, |
| | HLPPRGSVPGDPVRIH |
| SEQ ID NO: 41 | N-myristylation site polypeptide, |
| | QDYLNGAVSGSVQATD |
| SEQ ID NO: 42 | N-myristylation site polypeptide, |
| | NGAVSGSVQATDRLMK |
| SEQ ID NO: 43 | N-myristylation site polypeptide, |
| | SQSFKGGNYAVELVND |
| SEQ ID NO: 44 | N-myristylation site polypeptide, |
| | GYVLGGGAICMELLTK |
| SEQ ID NO: 45 | N-myristylation site polypeptide, |
| | ARVQFGANKSQYSLTR |
| SEQ ID NO: 46 | Amidation site polypeptide, |
| | EEEPAEGKKSEDDG |
| SEQ ID NO: 47 | UBC Domain of RATL1d6 |
| SEQ ID NO: 48 | RATL1d6 sense primer- |
| | expression profiling, |
| | 5′-aggatcatctccgacctgtg-3′ |
| | (Example 13) |
| SEQ ID NO: 49 | RATL1d6 antisense primer- |
| | expression profiling, |
| | 5′-caagggttgatccagcatct-3′ |
| | (Example 13) |
| SEQ ID NO: 50 | Promoter region of attacinD AMP |
| | forward primer (Example 14), |
| | 5′-atgaggcttggatcagcttt-3′ |
| SEQ ID NO: 51 | Promoter region of attacinD AMP |
| | reversed primer (Example 14), |
| | 5′-cctgaagcctgacattccat-3′ |
| SEQ ID NO: 52 | Forward primer-dsDNA, |
| | 5′-actgcagccgattcattaatg-3′ |
| | (Example 14) |
| SEQ ID NO: 53 | Reverse primer-dsDNA, |
| | 5′- |
| | gaattaatacgactcactatagggagatat |
| | catacacatacgatttag-3′ |
| | (Example 14) |
| SEQ ID NO: 54 | Forward primer-dsDNA, |
| | 5′- |
| | gaattaatacgactcactatagggagacat |
| | gattacgccaagctcgaa-3′ |
| | (Example 14) |
| SEQ ID NO: 55 | Reverse primer-dsDNA, |
| | 5′-tgtaaaacgacggccagtgaa-3′ |
| | (Example 14) |

The contents of all patents, patent applications, published PCT applications and articles, books, references, reference manuals and abstracts cited herein are hereby incorporated by reference in their entirety to more fully describe the state of the art to which the invention pertains.

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings.

What is claimed is:

1. An isolated polynucleotide selected from the group consisting of:
(a) an isolated polynucleotide encoding a ubiquitin conjugating enzyme homologue comprising the amino acid sequence as set forth in SEQ ID NO:2, or a fragment thereof;
(b) an isolated polynucleotide comprising SEQ ID NO:1;
(c) an isolated polynucleotide, or fragment thereof, encoding a ubiquitin conjugating enzyme amino acid sequence having at least 80% sequence identity with the sequence of SEQ ID NO:2;
(d) an isolated polynucleotide having the nucleic acid sequence of ATCC Accession No. PTA-3745;
(e) an isolated polynucleotide having the nucleic acid sequence according to nucleotides 517 to 1782 of SEQ ID NO:1, wherein said nucleotides encode a polypeptide of SEQ ID NO:2 minus the start codon;
(f) an isolated polynucleotide having the nucleic acid sequence according to nucleotides 520 to 1782 of SEQ ID NO:1, wherein said nucleotides encode a polypeptide of SEQ ID NO:2 including the start codon;
(g) an isolated polynucleotide which is fully complementary to the polynucleotide according to (a) through (f).

2. A hybridization probe comprising the polynucleotide according to claim 1.

3. A composition comprising the isolated polynucleotide according to claim 1.

4. An expression vector containing the polynucleotide according to claim 1.

5. A host cell containing the expression vector according to claim 4.

6. A substantially purified ubiquitin conjugating (UBC) enzyme polypeptide selected from the group consisting of:
(a) a ubiquitin conjugating enzyme polypeptide having the amino acid sequence as set forth in SEQ ID NO:2;
(b) a ubiquitin conjugating enzyme polypeptide comprising an amino acid sequence having at least 80% sequence identity to the sequence set forth in SEQ ID NO:2;
(c) a polypeptide according to (a), wherein the amino acid sequence differs from SEQ ID NO:2 only by conservative substitutions;
(d) a polypeptide according to (a), wherein the amino acid sequence has at least 90% sequence identity to the sequence set forth in SEQ ID NO:2;
(e) an isolated ubiquitin conjugating enzyme polypeptide encoded by the nucleic acid sequence of ATCC Accession No. PTA-3745;
(f) an isolated polypeptide having the amino acid sequence according to amino acids 2 to 422 of SEQ ID NO:2, wherein said amino acid encode a polypeptide of SEQ ID NO:2 minus the start methionine;
(g) an isolated polypeptide having the amino acid sequence according to amino acids 1 to 422 of SEQ ID NO:2, wherein said amino acid encode a polypeptide of SEQ ID NO:2 including the start methionine;
(h) an isolated polypeptide having the transmembrane domain region set forth in SEQ ID NO:17; and
(i) a substantially purified fragment of the ubiquitin conjugating enzyme polypeptide according to any one of (a) to (h).

7. A substantially purified ubiquitin conjugating enzyme fusion protein comprising an amino acid sequence having at least 80% sequence identity to the sequence as set forth in SEQ ID NO:2 and an amino acid sequence of an Fc portion of a human immunoglobulin protein.

8. A pharmaceutical composition comprising the polypeptide according to claim 6, or a functional portion thereof, and a pharmaceutically acceptable diluent, carrier, or excipient.

9. A pharmaceutical composition comprising the fusion protein according to claim 7, and a pharmaceutically acceptable diluent, carrier, or excipient.

10. A purified antibody which binds specifically to the polypeptide according to claim 6, or an antigenic epitope thereof.

11. A method of screening for candidate compounds capable of binding to and/or modulating activity of a ubiquitin conjugating enzyme, comprising:
(a) contacting a test compound with a substantially or partially purified polypeptide according to claim 6; and
(b) selecting as candidate compounds those test compounds that bind to and/or modulate activity of the polypeptide.

12. The method according to claim 11, wherein the candidate compounds are small molecules, therapeutics, biological agents, or drugs.

13. A method of treating a cancer or tumor, comprising administering an antagonist or inhibitor of the ubiquitin conjugating enzyme polypeptide according to claim 6 in an amount effective to block ubiquitination of a tumor suppressor gene in cancer or tumor cells.

14. A method of suppressing the immune response in a subject requiring said suppression, comprising administering a modulator of the ubiquitin conjugating enzyme polypeptide according to claim 6 in an amount effective to cause immunosuppression.

15. A method of treating an immune or neuronal disorder in a mammal comprising administering the ubiquitin conjugating enzyme polypeptide according to claim 6 in an amount effective to treat the immune or neuronal disorder.

16. A method of treating a cancer or tumor, an immune disorder, a lymphoproliferative disorder, or a neurodegenerative disorder, comprising: administering to an individual in need of treatment or therapy an antagonist or inhibitor of the ubiquitin conjugating enzyme polypeptide according to claim 6 in an amount effective to treat the disorder by blocking ubiquitination of a tumor suppressor gene in a cancer or tumor cell, a lymphoid cell, or a cell of the nervous system.

17. A method of treating cancers or tumors, immune disorders, lymphoproliferative disorders, or neurodegenerative disorders, comprising: administering to an individual in need of treatment or therapy an agonist of the ubiquitin conjugating enzyme polypeptide according to claim 6 in an amount effective to treat the disorder.

18. A kit for hybridization comprising the isolated polynucleotide according to claim 1, and instructions for use.
